(19) United States
(12) Patent Application Publication
(10) Pub. No.: US 2025/0368958 A1
(43) Pub. Date: Dec. 4, 2025

(54) CAR-EXPRESSING PLURIPOTENT STEM CELL-DERIVED NEUTROPHILS LOADED WITH DRUG NANOPARTICLES AND USES THEREOF

(71) Applicant: Purdue Research Foundation, West Lafayette, IN (US)

(72) Inventors: Xiaoping Bao, West Lafayette, IN (US); Qing Deng; Yun Chang, West Lafayette, IN (US)

(21) Appl. No.: 18/875,094

(22) PCT Filed: Jun. 14, 2023
(86) PCT No.: PCT/US2023/068456

§ 371 Date: Dec. 13, 2024

Related U.S. Application Data
(60) Provisional application No. 63/351,906, filed on Jun. 14, 2022.
(60) Provisional application No. 63/416,026, filed on Oct. 14, 2022.

(51) Int. Cl. C12N 5/0787; A61K 9/51; A61K 31/495; A61K 47/69; A61P 35/00; C07K 14/435; C07K 14/715; C07K 14/73; C07K 14/735

(52) U.S. Cl. CPC C12N 5/0642; A61K 9/5115; A61K 31/495; A61K 47/6901; A61P 35/00; C07K 14/43522; C07K 14/70514; C07K 14/70535; C07K 14/7153; A61K 2239/15; A61K 2239/21; C07K 2319/02; C07K 2319/03; C12N 2506/45; C12N 2510/00

(57) ABSTRACT

Chimeric antigen receptor (CAR)-expressing neutrophils loaded with nanoparticles comprising a drug; and a method of treating cancer or other disorders in a subject comprising administering to the subject a therapeutically effective amount of the CAR-expressing neutrophils.

### PRIORITY

This application is related to and claims the priority benefit of U.S. Provisional Patent Application No. 63/351,906 filed Jun. 14, 2022 and U.S. Provisional Patent Application No. 63/416,026 filed Oct. 14, 2022. The content of the aforementioned applications are hereby incorporated by reference in their entireties into this disclosure.

### TECHNICAL FIELD

The present disclosure relates to chimeric antigen receptor (CAR)-expressing neutrophils, which have been differentiated from pluripotent stems cells engineered to express the CAR, that are loaded with nanoparticles comprising a drug, and methods of using the neutrophils to treat cancer and other disorders.

### STATEMENT OF SEQUENCE LISTING

A computer-readable form (CRF) of the Sequence Listing is submitted concurrently with this application. The file, 69903-03_SeqListing.xml was generated on Jun. 13, 2023, file size: 54 kilobytes, which is herein incorporated by reference in its entirety. The content of the computer-readable form is the same and the information recorded in computer readable form is identical to the written sequence listing.

### BACKGROUND

Glioblastoma (GBM) is one of the most aggressive and lethal solid tumors in humans. GBM is characterized by a poor prognosis with a high tendency of recurrence, a short lifespan, and a high mortality rate. Yang et al., Synergistic immunotherapy of glioblastoma by dual targeting of IL-6 and CD40, Nature Communications 12: 3424 (2021); Lim et al., Current state of immunotherapy for glioblastoma, Nat'l Review Clinical Oncology 15:422-442 (2018). Therapeutic efficacies of both surgery and chemotherapeutic drugs can be largely hindered by the special fine brain structure and physiological blood-brain barrier (BBB) or blood-brain-tumor barrier (BBTB).

Agliardi et al., Intratumoral IL-12 delivery empowers CAR-T cell immunotherapy in a preclinical model of glioblastoma, Nature Communications 12: 444 (2021; Nemeth et al., Neutrophils as emerging therapeutic targets, Nature Reviews Drug Discovery 19: 253-275 (2020); Subhan & Torchilin, Neutrophils as an emerging therapeutic target and tool for cancer therapy, Life Sciences 285(15): 119952 (2021).

Due to their native capacity to migrate towards inflamed sites and traverse the BBB/BBTB to infiltrate solid tumors, neutrophil-mediated delivery of nanoparticulated chemotherapeutic drugs has been investigated to enhance targeted drug delivery to brain tumors for improved therapeutic efficacy. Xue et al., Neutrophil-mediated anticancer drug delivery for suppression of postoperative malignant glioma recurrence, Nature Nanotechnology 12: 692-700 (2017); Chu et al., Photosensitization Priming of Tumor Microenvironments Improves Delivery of Nanotherapeutics via Neutrophil Infiltration, Advanced Materials 29(27): 1701021 (2017); Wu et al., MR imaging tracking of inflammation-activatable engineered neutrophils for targeted therapy of surgically treated glioma, Nature Communications 9: 4777 (2018). However, an invasive surgical resection of tumor or tumor microenvironment (TME) priming is needed to induce additional inflammation for neutrophil recruitment before neutrophil/chemotherapeutic administration, leading to limited neutrophil recruitment in tumor sites beyond inflamed surgical margins. Osuka & Van Meir, Cancer therapy: Neutrophils traffic in cancer nanodrugs, Nature Nanotechnology 12: 616-618 (2017). Furthermore, neutrophil-mediated chemotherapeutics are mostly enriched in the spleen.

While necrosis had not been observed in the major organs of experimental brain tumor-bearing mice, there are concerns regarding off-target tissue toxicity or even systemic toxicity in human patients. Lin et al., Roles of Neutrophils in Glioma and Brain Metastases, Frontiers in Immunology 12 (2021). In addition, the innate immunity and plasticity of neutrophils against various pathogens, including GBM, were not well-explored or were ignored in these studies. Lin et al. (2021), supra; Fridlender et al., Polarization of tumor-associated neutrophil phenotype by TGF-beta: “N1” versus “N2” TAN, Cancer Cell 16(3): 183-194 (2009); Blaisdell et al., Neutrophils Oppose Uterine Epithelial Carcinogenesis via Debridement of Hypoxic Tumor Cells, Cancer Cell 28(6): 785-799 (2015); Mahiddine et al., Relief of tumor hypoxia unleashes the tumoricidal potential of neutrophils, J Clinical Investigation 130(1): 389-403 (2020); Yan et al., Human polymorphonuclear neutrophils specifically recognize and kill cancerous cells, Oncoimmunology 3(7) (2014).

Given that previous studies have focused on mouse neutrophils, the feasibility and safety of using human neutrophils in drug delivery remains elusive since massive neutrophil extraction from pre-surgical patients may lead to neutropenia or other risks. In addition, the intrinsic anti-tumor activities of naïve neutrophils need to be explored and, if possible, boosted to achieve an optimized therapeutic efficacy when used as a drug carrier in combination with chemotherapeutics.

Circulating neutrophils in the blood home to the hypoxic TME, where they become heterogenous tumor-associated neutrophils (TANs), an essential component of immunosuppressive TME that contributes to cancer progression and therapeutic resistance of tumors. Lin et al. (2021), supra; Jaillon et al., Neutrophil diversity and plasticity in tumour progression and therapy, Nature Reviews Cancer 20: 485-503 (2020). Similar to macrophages, anti-tumor N1 and pro-tumor N2 phenotypes of TANs have been found within the hypoxic TME. Li et al., Research Progress About Glioma Stem Cells in the Immune Microenvironment of Glioma, Frontiers in Pharmacology 12 (2021); Gieryng et al., Immune microenvironment of gliomas, Laboratory Investigations 97(5): 498-518 (2017); Jung et al., Tumor cell plasticity, heterogeneity, and resistance in crucial microenvironmental niches in glioma, Nature Communications 12: 1014 (2021); Dunn et al., Sonabend, Emerging immunotherapies for malignant glioma: From immunogenomics to cell therapy, Neuro-Oncology 22(10): 1425-1438 (2020).

Various therapeutic strategies have been developed to target neutrophils directly with a focus on neutrophil depletion or inhibition, leading to several clinical trials (e.g., CCR5 inhibitor Maraviroc in NCT03274804). Lin et al. (2021), supra; Yee et al., Neutrophil-induced ferroptosis promotes tumor necrosis in glioblastoma progression, Nature Communications 11: 5424 (2020). The direct application of untreated neutrophils as a nanocarrier, however, may pose an additional risk for cancer patients in which drug-trafficking neutrophils can be reprogrammed to the immunosuppressive pro-tumor N2 phenotype within the TME after homing to tumor sites. Fridlender et al. (2009), supra; Sagiv et al., Phenotypic diversity and plasticity in circulating neutrophil subpopulations in cancer, Cell Reports 10(4): 562-573 (2015).

Chimeric antigen receptor (CAR) modifications have significantly enhanced anti-tumor activities of immune T or natural killer (NK) cells, though their efficacy in solid tumors is still limited due in part to their relatively low trafficking and tumor penetration ability. Li et al., Human iPSC-Derived Natural Killer Cells Engineered with Chimeric Antigen Receptors Enhance Anti-tumor Activity, Cell Stem Cell 23(2): 181-192 (2018); Kim et al., High-affinity mutant Interleukin-13 targeted CAR T cells enhance delivery of clickable biodegradable fluorescent nanoparticles to glioblastoma, Bioactive Materials 5(3): 624-635 (2020); Nguyen et al., A novel ligand delivery system to non-invasively visualize and therapeutically exploit the IL13Rα2 tumor-restricted biomarker, Neuro-Oncology 14(10): 1239-1253 (2012); Wang et al., Chlorotoxin-directed CAR T cells for specific and effective targeting of glioblastoma, Science Translational Medicine 12(533) (2020). While efficacious therapeutics, such as emerging CAR-T cells and chemotherapeutics, have been developed to treat various cancers, their efficacy in GBM treatment has been largely hindered by the BBB or BBTB. Thus, a much safer and more effective human neutrophil-mediated biomimetic drug delivery system that mainly rests on the natural chemo-attractant activity of GBM is urgently needed.

### SUMMARY

Chimeric antigen receptor (CAR)-expressing neutrophils loaded with nanoparticles comprising a drug are provided. The CAR-expressing neutrophils can be, and desirably have been, differentiated from pluripotent stem cells (PSCs) engineered to express the CAR. The PSCs can be human PSCs (hPSCs). The hPSCs can comprise human embryonic stem cells (hESCs) and/or induced pluripotent stem cells (iPSCs).

The nanoparticles can comprise one or more of rough silica nanoparticles, cytosine arabinoside-based liposomes (e.g., DepoCyt®), polyamidoamine (PAMAM) dendrimer-albumin nanoparticles, and/or fullerene (e.g., gadofullerenol/fullerenol). The rough silica nanoparticles can be biodegradable mesoporous organic silica.

The drug can be a prodrug (e.g., preclinical or clinical), a chemotherapeutic drug, or a radiosensitizer. The prodrug can be activated by hypoxic conditions, acidic pH, an enzyme (e.g., horseradish peroxidase), or irradiation. The drug can be tirapazamine, temozolomide, climacostol, or indole-3-acetic acid, for example. The drug can be selected from the group consisting of everolimus, bevacizumab, belzutifan, carmustine, naxitamab-gqgk, and lomustine.

The CAR of the CAR-expressing neutrophils can comprise a neutrophil-specific transmembrane domain. The neutrophil-specific transmembrane domain can be a TLR4 polypeptide, a TLR2 polypeptide, a MET polypeptide, a granulocyte colony stimulating factor receptor (G-CSFR), a Myd88 polypeptide, a TRIF polypeptide, a Syk peptide, a CD40 polypeptide, CD32a, Dectin-1, a IL-6 receptor (IL6R), an Fe Epsilon Receptor Ig (FCERIG) polypeptide, a toll-like receptor 7 (TLR7), or a CD16 transmembrane aa CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a natural killer group 2D (NKG2D), Dectin-1, or CD16.

The CAR can comprise a 36-amino acid glioblastoma (GBM)-targeting chlorotoxin peptide, a CD4 transmembrane domain, and a CD3ζ intracellular domain. The CAR can comprise a 36-amino acid GBM-targeting chlorotoxin peptide, a C32a transmembrane domain, and a CD3ζ intracellular domain. The CAR can comprise a 36-amino acid GBM-targeting chlorotoxin peptide, either of a CD32a transmembrane domain or a CD16 transmembrane domain, and a CD3ζ intracellular signaling domain. The CAR can comprise a 36-amino acid GBM-targeting chlorotoxin peptide, either of a CD32a transmembrane domain or a CD16 transmembrane domain, and either of a CD32aγ intracellular signaling domain or a CD16 intracellular signaling domain and, in various embodiments, can further comprise an additional CD3ζ intracellular signaling domain. The CAR of the CAR-expressing neutrophils can comprise a 36-amino acid GBM-targeting chlorotoxin peptide; either a CD32a transmembrane domain or a CD16 transmembrane domain; and either a CD32aγ intracellular signaling domain or a CD16 intracellular signaling domain.

The CAR can comprise a 36-amino acid GBM-targeting chlorotoxin peptide, a NKG2D transmembrane domain, a 2B4 co-stimulatory domain, and a CD3ζ intracellular signaling domain.

The CAR can comprise an IL-13 receptor α 2 (IL-13Rα2)-targeted quadruple mutant IL-13 (TQM13) T-CAR, GD2-targeting scFV, HER2-targeting scFV, a vIII mutant epidermal growth factor receptor (EGFRvIII)-targeting scFV, or other glioma-targeting scFVs. a CD4 transmembrane domain, and a CD3ζ intracellular signaling domain.

The neutrophils can have an anti-tumor N1 phenotype. The neutrophils can exhibit anti-GBM activity in a hypoxic tumor microenvironment.

The CAR of the CAR neutrophils can be encoded by SEQ ID NO: 2, SEQ ID NO: 3, SEQ ID NO: 4, or a functional variant of SEQ ID NO: 2, 3, or 4.

Neutrophil-specific CAR constructs are also provided. In certain embodiments, a neutrophil-specific CAR construct comprises one or more sequences that encode: a disease-targeting peptide, a neutrophil-specific transmembrane domain, and an intracellular domain.

The neutrophil-specific transmembrane domain can be CD32a. The neutrophil-specific transmembrane domain can be CD4. The neutrophil-specific transmembrane domain can be NKG2D, Dectin-1, an IL-6 receptor, or CD16. The disease-targeting peptide can be a 36-amino acid GBM-targeting chlorotoxin. The intracellular domain can be a CD3ζ signaling domain. The intracellular domain can be either a CD32aγ intracellular signaling domain or a CD16 intracellular signaling domain. In certain embodiments, the CAR further comprises a sequence that encodes a (i.e. an additional) CD3ζ intracellular signaling domain. The construct can further comprise one or more sequences that encode a 2B4 co-stimulatory domain and, for example, the intracellular domain can be a CD3ζ intracellular signaling domain.

In certain embodiments of the CAR construct, the transmembrane domain is a CD4 transmembrane domain and the intracellular domain is a CD3ζ intracellular signaling domain; and the CAR further comprises one or more sequences that encode: an IL-13 receptor α 2 (IL-13Rα2)-targeted quadruple mutant IL-13 (TQM13) T-CAR, GD2-targeting single chain variable fragment (scFV), a human epidermal growth factor receptor 2 (HER2)-targeting scFV, a vIII mutant epidermal growth factor receptor (EGFRvIII)-targeting scFV, or other glioma-targeting scFV.

In certain embodiments, the CAR construct comprises SEQ ID NO: 2, SEQ ID NO: 3, SEQ ID NO: 4, or is a functional variant of SEQ ID NO: 2, 3 or 4.

Engineered neutrophil cell lines are also provided. In certain embodiments, the engineered neutrophil cell line comprises any CAR described herein. For example, the CAR can comprise: a disease-targeting peptide, a neutrophil-specific transmembrane domain, and an intracellular domain. The neutrophil-specific transmembrane domain can be CD32a. The disease-targeting peptide can be 36-amino acid GBM-targeting chlorotoxin. The neutrophil-specific transmembrane domain can be a CD4 transmembrane domain. The transmembrane domain can be a NKG2D, Dectin-1, an IL-6 receptor, or CD16. The neutrophil-specific transmembrane domain can be either a CD32a transmembrane domain or a CD16 transmembrane domain. The intracellular domain can be an CD3ζ intracellular signaling domain. In certain embodiments, the intracellular domain comprises a CD32aγ intracellular signaling domain or a CD16 intracellular signaling domain. The CAR of the engineered neutrophil cell line can comprise either a CD32a transmembrane domain or a CD16 transmembrane domain, and either a CD32aγ intracellular signaling domain or a CD16 intracellular signaling domain. The CAR can further comprise a CD3ζ intracellular signaling domain. The CAR can further comprise a 2B4 co-stimulatory domain.

In certain embodiments of the engineered neutrophil cell line, the disease-targeting peptide is a 36-amino acid GBM-targeting chlorotoxin; the neutrophil-specific transmembrane domain is a CD4 transmembrane domain; the intracellular domain is a CD3ζ intracellular signaling domain; and the CAR further comprises an IL-13Rα.2-TQM13 T-CAR, GD2-targeting scFV, a HER2-targeting scFV, an EGFRvIII-targeting scFV, or other glioma-targeting scFV.

Still further, pharmaceutical compositions are provided. The pharmaceutical composition can comprise any of the CAR-expressing neutrophils hereof or neutrophils from any of the engineered neutrophil cell lines hereof; and a pharmaceutically acceptable carrier and/or diluent. The pharmaceutical composition can further comprise a pharmaceutically acceptable excipient.

In certain embodiments, uses of any of the CAR-expressing neutrophils hereof, engineered neutrophil cell lines hereof, or pharmaceutical compositions hereof in the manufacture of a medicament for the treatment of a disease in a subject are provided. The disease can be cancer (e.g., GLB). In certain embodiments, the disease is a neurological disorder (e.g., Parkinson's disease or Alzheimer's disease).

A method of treating cancer in a subject is also provided. In certain embodiments, the method comprises administering to a subject a first therapy comprising therapeutically effective amount of a population of any of the CAR-expressing neutrophils hereof, a population of neutrophils from any of the engineered neutrophil cell lines hereof, or a pharmaceutical composition hereof; whereupon the subject is treated for cancer. The cancer can be a brain cancer, such as GLB. The cancer can be prostate cancer.

Administering the first therapy can comprise a delivery route selected from the group consisting of intravenous, intraperitoneal, intramuscular, intradermal, subcutaneous, intrathecal, intraosseous, and a combination of any of the foregoing. The first and second therapies can be administered sequentially and/or alternatively.

The method can further comprise administering a second therapy to the subject. The second therapy can vary depending on the type of disease to be treated. In certain embodiments, the second therapy is and/or comprises surgical removal of cancerous cells from the subject. Additionally or alternatively, the second therapy can comprise a chemotherapy, radiotherapy, or both.

In certain embodiments, the method of treating a cancer further comprises imaging a cancer in the subject prior to or during administering the first and/or second therapies.

Methods of delivering a therapeutic agent to a targeted location in a subject with a disease are also provided. In certain embodiments, such a method comprises administering to the subject a first therapy comprising a therapeutically effective amount of: a population of any of the CAR-expressing neutrophils hereof; a population of neutrophils from any of the engineered neutrophil cell lines hereof: or any pharmaceutical composition hereof, wherein the targeted location is across a blood brain barrier of the subject relative to the site of administration.

The disease can be a cancer, for example, a brain cancer. The disease can be a GLB. The disease can be a neurological disorder. The neurological disorder can involve protein aggregation of proteins prone to aggregate. The neurological disorder can be a tauopathy. The neurological disorder can be Alzheimer's disease or Parkinson's disease.

In certain embodiments, the method of delivering a therapeutic agent to a targeted location further comprises administering a second therapy to the subject.

The second therapy can comprise surgical removal of cancerous cells from the subject (e.g., wherein the disease is a cancer). The second therapy can comprise a chemotherapy, radiotherapy, or both. The method can further comprise imaging the targeted location in the subject prior to or during administering the first and/or second therapies. The targeted location can comprise brain tissue. The second therapy can comprise a microtubule-stabilizing agent. The first and second therapies can be administered sequentially and/or alternatively.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIGS. 1A and 1B are schematic illustrations of enhanced anti-glioblastoma efficacy using combinatory immunotherapy of chimeric antigen receptor (CAR)-neutrophils and tumor microenvironment responsive nano-prodrug, with FIG. 1A showing human pluripotent stem cells engineered with CARs and differentiated into CAR-neutrophils for targeted nano-prodrug delivery, and FIG. 1B showing CAR-neutrophils loaded with rough silica nanoparticles containing hypoxia-targeting tirapazamine (TPZ) (CAR-neutrophil@R—SiO₂-TPZ NPs) exhibit significantly enhanced anti-glioblastoma efficacy via combinatory CAR-neutrophil-mediated direct tumor killing and tumor microenvironment (TME) responsive nano-prodrugs.

FIGS. 2A-2F show results of screening CAR structures with enhanced neutrophil-mediated anti-tumor activities, where FIG. 2A is a schematic of various CAR structures, FIG. 2B is a schematic of CAR #1 construct and targeted knock-in strategy at the AAVS1 safe harbor locus of human pluripotent stem cells (hPSCs) (the vertical arrow indicating the AAVS1 targeting sgRNA and the horizontal arrows indicating primers for assaying targeting efficiency and homozygosity, FIG. 2C is a schematic of optimized neutrophil differentiation from hPSCs under chemically defined conditions, FIG. 2D shows graphical data from cytotoxicity assays against U87MG glioblastoma cells that were performed at different ratios of neutrophil-to-tumor target using indicated neutrophils (data are represented as mean±s.d. of five independent replicates, *p<0.05), and FIGS. 2E and 2F show reactive oxygen species (ROS) generation (FIG. 2E) and ELISA analysis of tumor necrosis factor alpha (TNFα) release (FIG. 2F) from different neutrophils after co-culturing with U87MG cells were determined.

FIGS. 3A-3E show data that CAR neutrophils, but not primary neutrophils, sustained superior anti-tumor activities under immunosuppressive tumor microenvironment, where FIG. 3A shows data from cytotoxicity assays against U87MG glioblastoma cells that were performed at different ratios of neutrophil-to-tumor target using CAR-neutrophils or peripheral blood (PB) neutrophils under indicated conditions, FIG. 3B shows ROS generation and FIG. 3C shows ELISA analysis of TNFα release from different CAR- or PB-neutrophils after co-culturing with U87MG cells were determined under indicated conditions, and FIGS. 3D and 3E show flow cytometry analysis of arginase and inducible nitric oxide synthetase (iNOS) expression on CAR- or PB-neutrophils under indicated conditions was shown in FIG. 3D and quantitated in FIG. 3E.

FIGS. 4A-4L show the preparation and characterization of hPSC CAR-neutrophils loaded with tirapazamine (TPZ)-containing SiO₂ nanoparticles, where FIGS. 4A and 4B show transmission electron microscope (TEM) (FIG. 4A) and energy dispersive spectroscopy (EDS) elemental mapping images (FIG. 4B) of rough SiO₂ nanoparticles, FIG. 4C shows data from a nitrogen adsorption-desorption isotherm of rough SiO₂ nanoparticles along with a Barrett-Joyner-Halenda (BJH) pore size distribution plot, FIGS. 4D and 4E show TPZ loading content in SiO₂ nanoparticles (FIG. 3D) and glutathione (GSH)-responsive TPZ release (FIG. 4E) measured at the indicated times, FIGS. 4F and 4G show fluorescence images (FIG. 4F) and flow cytometry analysis data (FIG. 4G) of neutrophils loaded with smooth and rough SiO₂-TPZ, FIG. 4H shows measured cellular SiO₂ content measured in hPSC-derived CAR-neutrophils, and FIGS. 4I-4M show cellular viability (FIG. 4I), transmigration (FIG. 4J), chemoattraction abilities (FIGS. 4K-4L), and ROS generation ability (FIG. 4M) of hPSC-derived CAR-neutrophils loaded with or without rough SiO₂-TPZ.

FIGS. 5A-5F show data supporting that CAR-neutrophils loaded with R—SiO₂-TPZ nanoparticles effectively kill glioblastoma cells, where FIG. 5A shows representative images of immunological synapses indicated by polarized F-actin accumulation at the interface between CAR-neutrophils and tumor cells at 6, 12 and 24 hours, supporting that R—SiO₂-TPZ nanoparticles released from CAR neutrophils upon tumor cell phagocytosis were up-taken by tumor cells, FIG. 5B is a schematic illustration of neutrophil-mediated antitumor cytotoxicity assay, FIGS. 5C-5E showing results from cytotoxicity studies against U87MG glioblastoma cells performed at different ratios of neutrophil-to-tumor target using indicated neutrophils at 24 hours (FIG. 5C), 48 hours (FIG. 5D), and 72 hours (FIG. 5E) (where the key in FIG. 5E applies to each graph if FIGS. 5C-5E), and FIG. 5F shows a heatmap resulting from a bulk RNA sequencing analysis performed on U87MG cells under various conditions, with expression levels of selected cytoplasm, membrane, oxidative stress, apoptosis, and proliferation-related genes in the indicated glioblastoma cells shown.

FIGS. 6A-6I show data from the functional evaluation of CAR-neutrophils loaded with R—SiO₂-TPZ nanoparticles using glioblastoma (GBM) microenvironment mimicking models in vitro, where FIG. 6A is a schematic of an in vitro tumor model of GBM with blood-brain-barrier (BBB), FIG. 6B shows data from a Transwell migration analysis of neutrophils at 12 hours, FIG. 6C shows data from an anti-GBM cytotoxicity study of indicated neutrophils where cytotoxicity was measured and quantified at 24 hours, FIG. 6D is an enzyme-linked immunoassay (ELISA) analysis of interleukin-6 (IL-6) and TNFα released from indicated neutrophils at 36 hours, FIG. 6E shows data from a second migration analysis of different neutrophils at 48 hours, FIG. 6F shows data from an anti-GBM cytotoxicity analysis of indicated neutrophils where cytotoxicity was measured and quantified at 60 hours, FIG. 6G is a schematic of a neutrophil-infiltrated three-dimensional (3D) tumor model in vitro, FIG. 6H shows representative fluorescent images of infiltrated neutrophils in the 3D tumor models (4′6-diamidino-2-phenylindole (DAPI) was used to stain the cell nuclear and CD45 was used to stain neutrophils; scale bars, 200 m), and FIG. 6I shows data related to the corresponding tumor-killing ability of indicated neutrophils measured and quantified using a cytotoxicity kit (data are represented as mean s.d. of five independent replicates, *p<0.05). The data in FIGS. 6B-6F are identified as follows: A—CAR hPSC-neutrophil@R-SiO₂-TPZ; B—CAR hPSC-neutorphil; C—PB neutrophil@R—SiO₂-TPZ; D—PB neutrophil; E—R—SiO₂-TPZ.

FIGS. 7A-7D relate to in vivo distribution studies of CAR neutrophil-delivered R—SiO₂-TPZ nanoparticles (NPs), where FIG. 7A is a schematic of intravenously administered Cy5-labeled CAR neutrophil@R—SiO₂ NPs and R—SiO₂ NPs for an in vivo cell tracking study, where 5×10⁵ luciferase (Luci)-expressing U87MG cells were stereotactically implanted into the right forebrain of NRG mice. After 4 days, mice were intravenously treated with phosphate-buffered saline (PBS), 5×10⁶ Cy5-labeled CAR neutrophil@R—SiO₂ NPs and R—SiO₂ NPs. Time-dependent biodistribution of Cy5+ neutrophils in whole body, brain, and other organs was determined and quantified by fluorescence imaging at the indicated hours (FIG. 7B), and FIG. 7C shows quantified data related to the biodistribution of CAR neutrophil(@R—SiO₂ NPs and R—SiO₂ NPs in mice at 24 hours post-injection, as analyzed by inductively coupled plasma-optical emission spectrometry (ICP-OES) based on Si element, with the data expressed as the percentage of injected dose per gram of tissue (% ID/g). FIG. 7D are representative fluorescence images of CD45 and SiO₂ in the indicated GBM xenografts isolated from tumor-bearing mice (scale bars, 100 μm).

FIGS. 8A-8E relate to the in vivo anti-tumor activities of combinatory CAR neutrophils and R—SiO₂-TPZ NPs assessed via intravenous injection. FIG. 8A is a schematic of intravenously administered PB neutrophils, PB neutrophils@R—SiO₂-TPZ NPs, R—SiO₂-TPZ NPs, CAR neutrophils, and CAR neutrophil@R—SiO₂-TPZ NPs for the in vivo tumor-killing study, where 5×10⁵ luciferase (Luci)-expressing U87MG cells were stereotactically implanted into the right forebrain of NRG mice. After 4 days, mice were intravenously treated with indicated neutrophils weekly for a month. FIG. 8B shows images of the NRG mice to determine time-dependent tumor burden and FIG. 8C shows the data of FIG. 8B quantified by bioluminescent imaging (BLI) at the indicated days (data are mean±s.d. for mice in (B) (n=5)). FIG. 8D is a Kaplan-Meier curve demonstrating survival of indicated experimental groups, and FIGS. 8E and 8F show graphical data related to released human TNFα and IL-6 in the peripheral blood (FIG. 8E) and the body weight (FIG. 8F) of different mouse groups, measured at the indicated days.

FIGS. 9A-9F relate to genotyping studies of CAR-engineered hPSCs. FIGS. 9A-9D are images of gels showing genotyping data of single cell-derived hPSC clones after puromycin selection for CAR #1 (FIG. 9A), CAR #2 (FIG. 9B), CAR #3 (FIG. 9C), and CAR #4 (FIG. 9D), where the expected polymerase chain reaction (PCR) product for a correctly targeted AAVS1 site was 991 bp (indicated by an arrow and “991 bp”), and an homozygosity assay was performed on the knock-in clones, and those without ˜240 bp PCR products were homozygous (indicated by an arrow and “204 bp”). FIGS. 9E and 9F show reverse transcriptase (RT)-PCR (FIG. 9E) and flow cytometry (FIG. 9F) analysis results of CLTX-IgG4 CAR expression on wildtype and CAR knock-in hPSCs.

FIG. 10 shows flow cytometry data of CAR-neutrophils derived from various hPSCs, where the plots show unstained control (black and shaded gray) and specific antibody (red and not shaded) histograms.

FIG. 11A shows graphical data related to the cytotoxicity ability of PB or CAR neutrophils against indicated tumor cells at an effector-to-target ratio of 10:1. For GBM cell killing, a U87MG cell line, primary adult GBM43, and pediatric SJ-GBM2 cells were employed. Data are represented as mean±s.d. of five independent replicates.

FIG. 11B shows quantified data related to the cell viability of CAR neutrophils when incubated with normal SVG p12 glial cells, H9 hPSCs, hPSC-derived mesoderm, endoderm and ectoderm at a neutrophil-to-target ratio of 10:1. Data are represented as mean±s.d. of five independent replicates.

FIG. 12A a TEM image of smooth (S—SiO₂) silica NPs.

FIG. 12B is nitrogen adsorption-desorption isotherm data of S—SiO₂ NPs and a BJH pore size distribution plot.

FIG. 12C is a graph of measured TPZ loading content in SiO₂ NPs.

FIG. 12D is a TEM image of rough (R—SiO₂) silica NPs.

FIG. 12E is a graph of the measured hydrodynamic sizes of S—SiO₂ NPs, S—SiO₂-TPZ NPs, R—SiO₂ NPs, and R—SiO₂ silica NPs.

FIG. 12F is a graph of the hydrodynamic sizes of TPZ containing R-SiO₂NPs (R—SiO₂-TPZ NPs) in the presence of 10 mM GSH and measured at the indicated time points.

FIG. 12G is a TEM image of R—SiO₂-TPZ NPs incubated with 10 mM GSH for 50 hours.

FIG. 13 is flow cytometry data of CD11b expression on CAR neutrophils before and after loading R—SiO₂-TPZ NPs.

FIG. 14 is data related to the number of immunological synapses formed between indicated neutrophils and tumor cells/normal somatic cells.

FIGS. 15A-15C relate to the stability of R—SiO₂-TPZ NPs within CAR-neutrophils. FIG. 15A is a schematic of an experimental design to investigate the stability of R—SiO₂-TPZ NPs within CAR neutrophils after loading. FIG. 15B is measured relative TPZ intensity before and after centrifugation. FIG. 15C is flow cytometry data of the cellular content of R—SiO₂-TPZ NPs within tumor cells after being cocultured with CAR-neutrophils@R—SiO₂-TPZ NPs.

FIG. 16A is a volcano map of bulk RNA-Seq analysis of U87MG cells under indicated treatments. FIGS. 16B and 16C show tumor lysis measurements of the indicated groups treated with 5 μM cytochalasin D (CytoD) or 5 mM N-acetylcysteine (NAC), respectively.

FIG. 17A is data related to measured amounts of the loading contents of temozolomide (TMZ) and JNJ64619187 in R—SiO₂ NPs.

FIG. 17B is a schematic of a neutrophil-infiltrated 3D tumor model.

FIG. 17C is a graph of the quantified, corresponding tumor-killing ability of the indicated neutrophils, which was measured and quantified using cytotoxicity kit (data represented as mean s.d. of five independent replicates).

FIG. 18 is H&E images of major organs collected at the end of treatment (scale bar, 200 μm).

FIG. 19A are schematic diagrams of positron emission topography (PSMA)-CAR design and knock-in strategy via Cas9-mediated homology-directed repair (HDR) at the endogenous AAVS1 safe harbor locus. PSMA-CAR can be composed of signal peptide, anti-PSMA J591 scFV or nanobody, IgG4-Fc (EQ), CD4 transmembrane (tm) and CD3ζ (CD3z).

FIG. 19B is images of gels resulting from the genotyping of a CAR knockin in hPSCs with a target efficiency of 12 clones from a total of 13 and 13 clones from a total of 15, respectively.

FIG. 19C are graphs of the cytotoxicity of CAR-neutrophils co-cultured with U87MG GBM and LNCaP prostate cancer cells at indicated cell ratios for 16 hours.

FIG. 19D is a schematic of a study protocol comprising anti-PSMA J591 CAR-neutrophils loaded with SiO₂-TPZ nanodrugs for enhanced antitumor cytotoxicity tested in a hypoxia tumor model in vitro, and data related to such cytotoxicity measured at the indicated times.

FIGS. 20A and 20B show anti-tumor activity of increased dosage frequencies of the CAR-neutrophils and R—SiO₂-TPZ NPs of the studies shown in FIGS. 8A-8F. FIG. 20AB is a Kaplan-Meier curve demonstrating survival of indicated experimental groups (n=5) (Kaplan-Meier curves were analyzed by the log-rank test).

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

Given their innate immunity against pathogens and native ability to cross physiological barriers, the present disclosure provides human neutrophils engineered with synthetic chimeric antigen receptors (CARs). The CAR-expressing neutrophils can provide improved direct anti-tumor cytolysis and enhanced noninvasive glioblastoma (GBM)-targeted delivery of nanoparticulated chemotherapeutics without additional inflammation-induced chemotaxis. Primary neutrophils are short-lived and cannot be genetically modified, which has conventionally limited their broad application in CAR-directed immunotherapy. Roberts et al., Antigen-specific cytolysis by neutrophils and NK cells expressing chimeric immune receptors bearing zeta or gamma signaling domains, J Immunology 161(1): 375-384 (1998). Human pluripotent stem cells (hPSCs), on the other hand, are more accessible to gene editing, can differentiate into neutrophils on a massive scale, and can provide an unlimited source of high-quality CAR-neutrophils for targeted immunotherapy under chemically defined, xeno-free conditions. Chang et al., Engineering chimeric antigen receptor neutrophils from human pluripotent stem cells for targeted cancer immunotherapy, Cell Reports 40(3) (2022).

The present disclosure harnesses the power of self-renewing hPSCs to allow for the production of unlimited de novo CAR-expressing human neutrophils to provide a powerful, bioinspired neutrophil-mediated drug delivery system using CAR-engineering. Neutorphil-specific CAR expression constructs are provided, as are CAR-expressing neutrophils. In certain embodiments, CAR-expressing neutrophils (or CAR neutrophils) loaded with nanoparticles (e.g., comprising a drug) are provided. The term “CAR neutrophils” means neutrophils that have been modified through molecular biological methods to express a CAR on the surfaces of the neutrophils. Use of engineered CAR-neutrophils as a nanocarrier (e.g., of a drug) is also provided. Such engineered CAR-neutrophils can have striking anti-tumor activities and, in certain embodiments, can be used to treat and, optionally target, various disease states, including GBM.

### CAR Constructs and CAR-Expressing Neutrophils

In view of the above, provided are neutrophil-specific CAR expression gene constructs and CAR-expressing neutrophils loaded with nanoparticles.

The CAR can be any suitable CAR as known in the art. CARs are artificially constructed hybrid receptor proteins or polypeptides that can graft an arbitrary specificity onto an immune effector cell, such as an NK cell. See, e.g., Sadelain et al., “The Basic Principles of Chimeric Antigen Receptor Design,” Cancer Discovery OF1-11 (2013). Non-limiting examples of complementarity-determining regions (CDRs) include, but are not limited to, CD19 (U.S. Pat. No. 7,446,190; and USPAPN 2013/0071414), HER2 (Ahmen et al., Clin Cancer Res (2010)), MUC16 (Chekmasova et al. (2011)), and PSMA (Zhong et al., Molec Ther 18(2); 413-410 (2010)). The CAR can have a pre-defined binding specificity to a desired target, such as matrix metallopeptidase 2 (MMP2), e.g., MMP2 on a glioma, such as a GBM. The terms “polypeptide,” “peptide,” and “protein” are used interchangeably herein (unless expressly stated otherwise) to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

Generally, a CAR is a fusion protein that can comprise a recognition region, co-stimulation domains, various signaling domains, costimulatory domains, spacers, and/or hinges. Desirably, the CAR is suitable for using the CAR neutrophils to treat cancer, e.g., the CAR binds a cell-surface antigen on a cancerous cell with high specificity.

In certain embodiments, the CAR is encoded by SEQ ID NO: 2 or a functional variant thereof.

In certain embodiments, the CAR is encoded by SEQ ID NO: 3 or a functional variant thereof. In certain embodiments, the CAR is encoded by SEQ ID NO: 4 or a functional variant thereof. The term “functional variant” refers to a CAR, a polypeptide, or a protein having substantial or significant sequence identity or similarity to the CAR, which functional variant retains the biological activity of the CAR of which it is a variant. Functional variants encompass, for example, those variants of the CAR (the parent CAR) that retain the ability to recognize target cells to a similar extent, the same extent, or to a higher extent, as the parent CAR. In reference to a nucleic acid sequence encoding the parent CAR, in some embodiments a nucleic acid sequence encoding a functional variant of the CAR is about 10% identical, about 25% identical, about 30% identical, about 50% identical, about 65% identical, about 80% identical, about 90% identical, about 95% identical, or about 99% identical to the nucleic acid sequence encoding the parent CAR.

The use of terms and phrases with regard to CAR binding specificity, such as “binds with specificity,” “binds with high affinity,” “binds with high specificity,” or “specifically” or “selectively” binds, indicates a binding reaction between a CAR, such as a CAR comprising CLTX, on a neutrophil and a target molecule, such as a protein (e.g., a receptor, an enzyme (e.g., MMP2), or a cell-surface marker) that is present on a targeted cell, such as a cancerous cell (e.g., a cell of which a tumor is comprised) or other diseased cell. Thus, under binding conditions that are conducive to, facilitate or otherwise promote binding of a CAR neutrophil with a target molecule that is present on a targeted cell, such as a cancerous cell or other diseased cell, such a CAR neutrophil does not bind significantly, if at all, to other molecules, such as proteins (e.g., receptors, enzymes, and cell-surface markers) present on normal, healthy cells. Specific binding or binding with high affinity can be at least 25% greater, more often at least 50% greater, most often at least 100% (2-fold) greater, normally at least ten times greater, more normally at least 20-times greater, and most normally at least 100-times greater than the binding of any other non-targeted molecule.

In certain embodiments, the CARs hereof bind with high specificity to a cancer cell (e.g., a brain cancer cell). In certain embodiments, the CARs hereof bind with high specificity to beta amyloid (e.g., for use in targeting/treating a neurological disorder). The CAR can be designed, for example, to target beta amyloid (e.g., soluble oligomers of the amyloid-β peptide (AβOs)). As the accumulation of AβOs in the brain has been implicated in synapse failure and memory impairment in Alzheimer's disease, targeting such AβOs can be useful in effectively delivering nanoparticle/drug cargo thereto to, for example, treat Alzheimer's disease and/or symptoms associated therewith. Selles et al., AAV-mediated neuronal expression of an scFv antibody selective for AB oligomers protects synapses and rescues memory in Alzheimer models, Molecular Therapies 31(2): 409-419 (2023). In certain embodiments, the CAR can comprise a NUsc1 single chain variable fragment (scFv) that selectively targets a population of AβOs in a subject.

CARs can include an extracellular domain, a transmembrane domain, and an intracellular domain. The extracellular domain can include an antigen binding/recognition region/domain and/or a scFv derived from an antibody for targeting. The antigen binding domain of the CAR can bind to a specific antigen, such as a cancer/tumor antigen (e.g., for the treatment of cancer), a pathogenic antigen, such as a viral antigen (e.g., for the treatment of a viral infection), or a CD antigen.

Examples of tumor antigens include, but are not limited to, carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, an antigen of a cytomegalovirus infected cell (e.g., a cell surface antigen), epithelial glycoprotein 2 (EGP2), epithelial glycoprotein 40 (EGP40), epithelial cell adhesion molecule (EpCAM), receptor tyrosine-protein kinase erb-B2, 3 or 4, folate-binding protein (FBP), fetal acetylcholine receptor (AChR), folate receptor α (FR(α), folate receptor β (FRβ), ganglioside G2 (GD2), ganglioside G3 (GD3), human epidermal growth factor receptor 2 (HER2), human telomerase reverse transcriptase (hTERT), interleukin 13 (IL-13) receptor subunit α2 (IL-13Rα2), κ light chain, kinase insert domain receptor (IDR), Lewis A (CA19.9), Lewis Y (LeY), L1 cell adhesion molecule (L1CAM), melanoma antigen family A1 (MAGE-A1), mucin 16 (Muc-16), mucin 1 (Muc-1), mesothelin (MSLN), a natural killer group 2D (NKG2D) ligand, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor receptor (VEGF-R, such as R2), and Wilms tumor protein (Wt-1).

Certain CARs are fusions of binding functionality (e.g., as a scFv derived from a monoclonal antibody) to a CD3-zeta (CD3ζ) transmembrane and endodomain. Such molecules can result in the transmission of a zeta signal in response to recognition by the recognition receptor binding functionality of its target. There are, however, many alternatives. By way of non-limiting example, an antigen recognition domain from native T cell receptor (TCR) alpha and beta single chains can be used as the binding functionality. Alternatively, receptor ectodomains (e.g., CD4 ectodomain) can be employed. All that is required of the binding functionality is that it can bind a given target with high affinity in a specific manner.

In certain embodiments, the CAR comprises a neutrophil-specific transmembrane domain. Examples of neutrophil-specific transmembrane domains include, but are not limited to, a TLR4 polypeptide, a TLR2 polypeptide, a MET polypeptide, a granulocyte colony stimulating factor receptor (G-CSFR), a Myd88 polypeptide, a TRIF polypeptide, a Syk peptide, a CD40 polypeptide, CD32a, Dectin-1, a IL-6 receptor (IL6R), an Fc Epsilon Receptor Ig (FCER1G) polypeptide, a toll-like receptor 7 (TLR7), or a CD16 transmembrane aa CD8 polypeptide, a CD28 polypeptide, an OX40 polypeptide, an ICOS polypeptide, a CTLA-4 polypeptide, a PD-1 polypeptide, a LAG-3 polypeptide, and a BTLA polypeptide. In certain embodiments, the neutrophil-specific transmembrane domain is CD32a. In certain embodiments, the neutrophil-specific transmembrane domain is CD4. In certain embodiments, the neutrophil-specific transmembrane domain is NKG2D, Dectin-1, an IL-6 receptor, or CD16.

The intracellular domain can comprise, for example, a CD3ζ polypeptide, and can further comprise at least one costimulatory signaling region comprising at least one costimulatory molecule. “Costimulatory molecule” refers to a cell surface molecule or receptor, other than an antigen receptor/ligand required for an efficient response of lymphocytes to antigen. The costimulatory signaling region can comprise a CD28 polypeptide, a 4-1BB polypeptide, an OX40 polypeptide, an ICOS polypeptide, a DAP-10 polypeptide, a PD-1 polypeptide, a LAG-3 polypeptide, a 2B4 polypeptide, a BTLA polypeptide, or a CTLA-4 polypeptide. Neutrophil-specific intracellular domains can also be used, which can be derived from FCERIG, CD32a, Dectin-1, IL6R, TLR4, or TLR2.

A CAR can be produced by any means known in the art, though preferably it is produced using recombinant DNA techniques. A nucleic acid sequence encoding the several regions of the CAR (i.e., a CAR construct) can be prepared and assembled into a complete coding sequence by standard techniques of molecular cloning and/or genetic engineering techniques (genomic library screening, PCR, primer-assisted ligation, site-directed mutagenesis, and gene editing techniques, such as CRISPR, etc.). Such techniques are generally known in the art (see, e.g., Sambrook et al., “Molecular Cloning: A Laboratory Manual,” 3^{rd} ed., Cold Spring Harbor Laboratory Press (2001); and Green and Sambrook, “Molecular Cloning: A Laboratory Manual,” 4^{th} ed., Cold Spring Harbor Laboratory Press (2012), both of which are specifically incorporated herein by reference for their teachings regarding same) and/or are exemplified herein.

The resulting coding region can be inserted into an expression vector for subsequent introduction into a recipient cell, such as a hPSC. The term “vector” means any nucleic acid that functions to carry, harbor, or express a nucleic acid of interest. Nucleic acid vectors can have specialized functions, such as expression, packaging, pseudotyping, or transduction. Vectors can also have manipulatory functions if adapted for use as a cloning or shuttle vector. The structure of the vector can include any desired form that is feasible to make and desirable for a particular use. Such forms can include, for example, circular forms such as plasmids and phagemids, as well as linear or branched forms. A nucleic acid vector can be composed of, or example, DNA or RNA, as well as contain partially or fully, nucleotide derivatives, analogs or mimetics. Such vectors can be obtained from natural sources, produced recombinantly or chemically synthesized.

By way of non-limiting examples, a plasmid or viral expression vector (e.g, a lentiviral vector, a retrovirus vector, sleeping beauty, and piggyback (transposon/transposase systems that include a non-viral mediated CAR gene delivery system)) can be prepared that encodes a fusion protein (i.e., CAR construct) comprising a recognition region, one or more co-stimulation domains, and an activation signaling domain, in frame and linked in a 5′ to 3′ direction.

CAR expression can be driven using any suitable promoter, such as exemplified herein. Examples of promoters include, but are not limited to, various constitutive and inducible promoters, such as a constitutive CAG promoter, an EF1a promoter, a UBC constitutive promoter, or a Teton-3G inducible promoter.

The placement of the recognition region in the fusion protein/construct will generally be such that display of the region on the exterior of the neutrophil is achieved. Where desired, the CARs can also include additional elements, such as a signal peptide (e.g.. CD8α signal peptide) to ensure proper export of the fusion protein to the cell surface. a transmembrane domain to ensure the fusion protein is maintained as an integral membrane protein (e.g., CD3ζ transmembrane domain), and a hinge domain that imparts flexibility to the recognition region and allows strong binding to the targeting moiety.

T and NK cell-specific CAR constructs have been widely used to enhance anti-tumor activities of T and NK cells, but neutrophil-specific CARs that improve anti-tumor functions of neutrophils have not been previously described. CD4ζ and CD4γ chimeric immune receptors were previously reported to enhance cytolysis of human neutrophils against HIVenv-transfected cells in vitro, but the lysis efficiency was only about 10% at an effector-to-target (E:T) ratio of 10:1. Roberts et al. (1998), supra. FcγRIIA (CD32a) is a low-affinity single-chain transmembrane receptor for monomeric immunoglobulin G (IgG) that highly expresses in neutrophils (30,000 to 60,000 molecules/cell), and its ligation induces Fcγ-dependent functions in neutrophils, such as release of granule contents, Ca²⁺ mobilization, anti-tumor cytotoxicity, and phagocytosis. Wang & Jonsson, Expression, role, and regulation of neutrophil Fcγ receptors, Frontiers Immunology 10 (2019); Nagarajan et al., Cell-specific, activation-dependent regulation of neutrophil CD32A ligand-binding function, Blood 95(3): 1069-1077 (2000).

In view of the prominent role of CD32a in the activation and function of neutrophils, in certain embodiments, CD32a-based CAR constructs are provided. Such CAR constructs have been screened and optimized, as described in the Examples below, with the results demonstrating that CD3ζ can mediate significantly better cytolysis than CD32aγ when expressed in hPSC-derived neutrophils. This may be in part due to the higher copies of ITAMs in CD3ζ than CD32aγ: three and one copies, respectively, and higher expression levels of ζ than γ on cell surface of neutrophils. Roberts et al. (1998), supra.

Similar to CD32a, FcγRIII (CD16b) is another low-affinity receptor for monomeric IgG and it expresses at a much higher level than CD32a on neutrophils. While cross-linking of CD16b only induced Ca²′ mobilization and degranulation, but not phagocytosis and cytolysis in neutrophils, a systematic comparison on the abilities of CD3ζ- and CD16bγ-CARs in triggering and enhancing anti-tumor functions of neutrophils may be warranted. Roberts et al. (1998), supra; Fanger et al., Cytotoxicity mediated by human Fc receptors for IgG, Immunology Today 10(3): 92-99 (1989); Wang & Jonsson (2019), supra.

In certain embodiments, the CAR constructs can include an antigen recognition domain that contains a disease-targeting peptide or fragment thereof, for example, directed against a tumor-associated antigen, for example, to facilitate binding affinity to the targeted disease site. In this manner, the neutrophils expressing the CAR constructs hereof can be used to target specific sites within a subject (e.g., a TME) which can reduce or obviate off-target effects of the drug cargo carried thereby. The antigen recognition domain of the CAR can be a whole antibody or an antibody fragment (e.g., scFv). The disease-targeting peptide can be, for example, a cancer-targeting peptide such as a GBM-targeting peptide or a functional fragment thereof. The disease-targeting peptide can be, for example, a fibrosis-targeting peptide such as fibroblast activation protein (FAP) or a functional fragment thereof. The terms “fragment of an antibody,” “antibody fragment,” “functional fragment of an antibody,” and “antigen-binding portion” are used interchangeably herein to mean one or more fragments or portions of an antibody that retain the ability to specifically bind to an antigen.

In certain embodiments, the CAR constructs are anti-GBM CAR constructs (e.g., anti-GBM chlorotoxin (CLTX)-CAR constructs) comprising T- or neutrophil-specific signaling domains. Such anti-GBM CAR constructs can be optimized, and genetically engineered hPSCs with the optimized CARs were screened as described in the Examples below, via CRISPR/Cas9-mediated gene knock-in at the AAVS1 safe harbor locus, and CAR constructs optimized for neutrophil-mediated tumor-killing were identified. Wang et al (2020), supra.

In certain embodiments, the CAR constructs encode a neutrophil-specific transmembrane domain. The neutrophil-specific transmembrane domain can be CD32a. The neutrophil-specific transmembrane domain can be CD4. The neutrophil-specific transmembrane domain can be CD32a. The neutrophil-specific transmembrane domain can be NKG2D, Dectin-1, IL-6 receptor, or CD16.

In various embodiments herein, such as when the CAR-expressing neutrophils are used to treat GBM, the CAR can comprise a GBM-targeting peptide, such as a 36-amino acid GBM-targeting chlorotoxin (CLTX) peptide. In such embodiments, the GBM-targeting peptide (e.g., the 36-amino acid GBM-targeting chlorotoxin peptide) is coupled with a CD4 transmembrane domain and a CD3ζ intracellular domain, such that the CAR comprises a 36-amino acid GBM-targeting chlorotoxin peptide, a CD4 transmembrane domain, and a CD3ζ intracellular domain. In other embodiments, the GBM-targeting peptide (e.g., the 36-amino acid GBM-targeting chlorotoxin peptide) is coupled with (i) either of a CD32a transmembrane domain or a CD16 transmembrane domain and (ii) a CD3ζ intracellular signaling domain, such that the CAR comprises (i) a 36-amino acid GBM-targeting chlorotoxin peptide, (ii) a CD32a transmembrane domain or a CD16 transmembrane domain, and (iii) a CD3ζ intracellular signaling domain. In yet other embodiments, the GBM-targeting peptide (e.g., the 36-amino acid GBM-targeting chlorotoxin peptide) is coupled with (i) either of a CD32a transmembrane domain or a CD16 transmembrane domain and (ii) a CD3ζ intracellular signaling domain, such that the CAR comprises (i) a 36-amino acid GBM-targeting chlorotoxin peptide, (ii) either of a CD32a transmembrane domain or a CD16 transmembrane domain, and (iii) a CD3ζ intracellular signaling domain. In yet still other embodiments, the GBM-targeting peptide (e.g., the 36-amino acid GBM-targeting chlorotoxin (CLTX) peptide) is coupled with (i) either of a CD32a transmembrane domain or a CD16 transmembrane domain and (ii) either of a CD32aγ intracellular signaling domain or a CD16 intracellular signaling domain, alone or in further combination with (iii) a CD3ζ intracellular signaling domain, such that the CAR comprises (i) a 36-amino acid GBM-targeting chlorotoxin peptide, (ii) either of a CD32a transmembrane domain or a CD16 transmembrane domain and (iii) either of a CD32aγ intracellular signaling domain or a CD16 intracellular signaling domain, alone or in further combination with (iv) a CD3ζ intracellular signaling domain. In yet even still other embodiments, the GBM-targeting peptide (e.g., the 36-amino acid GBM-targeting chlorotoxin peptide) is coupled with a NKG2D transmembrane domain, a 2B4 co-stimulatory domain, and a CD3ζ intracellular signaling domain, such that the CAR comprises a 36-amino acid GBM-targeting chlorotoxin peptide, a NKG2D transmembrane domain, a 2B4 co-stimulatory domain, and a CD3ζ intracellular signaling domain.

The CAR construct can comprise SEQ ID NO: 2 or a functional variant thereof. The CAR construct can comprise SEQ ID NO: 3 or a functional variant thereof. The CAR construct can comprise SEQ ID NO: 4 or a functional variant thereof. The term “functional variant” refers to a CAR, a polypeptide, or a protein having substantial or significant sequence identity or similarity to the CAR, which functional variant retains the biological activity of the CAR of which it is a variant.

Functional variants encompass, for example, those variants of the CAR (the parent CAR) that retain the ability to recognize target cells to a similar extent, the same extent, or to a higher extent, as the parent CAR. In reference to a nucleic acid sequence encoding the parent CAR, in some embodiments a nucleic acid sequence encoding a functional variant of the CAR is about 10% identical, about 25% identical, about 30% identical, about 50% identical, about 65% identical, about 80% identical, about 90% identical, about 95% identical, or about 99% identical to the nucleic acid sequence encoding the parent CAR.

In various other embodiments, such as when the CAR-expressing neutrophils are used to treat cancer, the CAR comprises an interleukin-13 (IL-13) receptor alpha-2 (IL-13Rα2)-targeted quadruple mutant IL-13 (TQMI 3) T-CAR, GD-2 targeting scFV, HER2-targeting scFV, vIII mutant epidermal growth factor receptor (EGFRvIII)-targeting scFV, or other glioma-targeting scFVs. CD4 transmembrane domain, and a CD3ζ intracellular signaling domain.

In certain embodiments, a (e.g., optimized) CAR construct is provided that comprises one or more sequences that encode a 36-amino acid GBM-targeting CLTX peptide, a CD4 transmembrane domain and a CD3ζ intracellular. In certain embodiments, a neutrophil-specific CAR construct comprises one or more sequences that encode a 36-amino acid GBM-targeting CLTX peptide, a NKG2D transmembrane domain, and an intracellular domain. The transmembrane domain can be, alternatively and in certain embodiments, either a CD32a transmembrane domain or a CD16 transmembrane domain.

The intracellular domain can be a CD3ζ signaling domain. The intracellular domain can be either a CD32aγ intracellular signaling domain or a CD16 intracellular signaling domain.

In certain embodiments, the CAR construct can further comprise one or more sequences that encodes a 2B4 co-stimulatory domain and, optionally, the intracellular domain can be a CD3ζ signaling domain. In certain embodiments of the CAR construct, the transmembrane domain is a CD4 transmembrane domain and the intracellular domain is a CD3ζ intracellular signaling domain, and the CAR further comprises one or more sequences that encode an IL-13 receptor α 2 (IL-13Rα2)-targeted quadruple mutant IL-13 (TQM13) T-CAR, GD2-targeting scFV, a HER2-targeting scFV, an EGFRvIII-targeting scFV, or other glioma-targeting scFV.

As previously described, the CAR constructs hereof can be used to produce stable CAR-expressing hPSCs, which can then be differentiated into massive neutrophils that express the CAR construct(s) (i.e., CAR-expressing neutrophils or CAR neutrophils, which are used interchangeably herein). In certain embodiments, the CAR-expressing neutrophils can have (and sustain) an anti-tumor N1 phenotype. Such neutrophils can exhibit anti-glioblastoma activity in a hypoxic tumor microenvironment (TME), for example.

Any suitable method as known in the art and exemplified herein can be used to deliver a CAR-encoding nucleic acid, such as a plasmid, into hPSCs. Examples of methods include, but are not limited to, nucleofection/electroporation, transfection via Lipofectamine Stem (ThermoFisher, STEM00001) or similar transfection reagents, or lentivirus, retrovirus, sleeping beauty, piggyback (transposon/transposase systems including a non-viral mediated CAR gene delivery system) or adeno-associated virus (AAV)-mediated delivery. Illustrated is a method using plasmids and CRISPR/Cas9, and transposons, ribonucleoproteins and double-stranded DNAs also can be used to integrate a CAR into the hPSC genome, such as at an AAVS1 safe harbor locus or a CLYBL locus.

The CAR-expressing neutrophils can be, and desirably have been, differentiated from pluripotent stem cells (PSCs) engineered to express the CAR. The use of “pluripotent” to describe stem cells refers to the ability of the cells to form all cell lineages of an organism—in this case, all cell lineages of a human. Pluripotency characteristics include, but are not limited to, morphology (e.g., small, round, high nucleus-to-cytoplasm ratio, notable presence of nucleoli, and inter-cell spacing), the potential for unlimited self-renewal, the expression of pluripotent stem cell markers (e.g., SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30, and/or CD50), the ability to differentiate into ectoderm, mesoderm, and endoderm, teratoma formation, and formation of embryoid bodies.

The PSCs can be engineered to express the CAR using methods known in the art and exemplified herein. While the use of a CRISPR/Cas9-mediated gene knock-in technique is exemplified herein to introduce a construct into the AAVS1 safe harbor locus to modify genetically hPSCs, any suitable genome editing method can be used. Genome editing, also referred to as genomic editing or genetic editing, is a type of genetic engineering in which DNA is inserted, deleted and/or replaced in the genome of a targeted cell. Targeted editing can be achieved through a nuclease-independent or nuclease-dependent approach. Nuclease-independent editing involves homologous recombination guided by homologous sequences flanking an exogenous polynucleotide to be inserted into a genome. Alternatively, specific endonucleases can be used to introduce double-stranded breaks into the DNA, which then undergo repair. CRISPR/Cas9 (clustered regular interspaced short palindromic repeats associated 9) is an RNA-guided nuclease. Other endonucleases include, but are not limited to, zinc-finger nucleases (ZFN) and transcription activator-like effector nucleases (TALEN). Another system is DICE (dual integrase cassette exchange), which utilizes phiC31 and Bxb1 integrases for targeted integration.

While the adeno-associated virus site 1 (AAVS1) safe harbor locus is exemplified herein, other sites for targeted integration include, but are not limited to, other safe harbor loci or genomic safe harbor (GSH), which are intragenic/extragenic regions of the human genome that, theoretically, are able to accommodate predictable expression of newly integrated DNA without adverse effects on the host cell or recipient organism. A useful safe harbor must permit sufficient transgene expression to yield desired levels of the vector-encoded protein or non-coding RNA. A safe harbor also must not predispose cells to malignant transformation or alter cellular functions. Ideally, the safe harbor locus is characterized by the absence of disruption of regulatory elements or genes, is an intergenic region in a gene dense area or a location at the convergence between two genes transcribed in opposite directions, keep distance to minimize the possibility of long-range interactions between vector-encoded transcriptional activators and the promoters of adjacent genes (in particular cancer-related and microRNA genes), and has ubiquitous transcriptional activity. The location should also be devoid of repetitive elements and conserved sequences and allow for easy design of primers for amplification. Suitable sites for human genome editing include, in addition to AAVS1, the chemokine (CC motif) receptor 5 gene locus, human orthologue of the mouse ROSA26 locus, the human orthologue of the mouse H11 locus, collagen loci, and HTRP loci. The selected site must be validated for specific integration and, oftentimes, the insertion strategy, promoter, gene sequence, and construct design require optimization.

Neutrophils can be differentiated from PSCs using methods known in the art and/or exemplified herein. The PSCs can be hPSCs. The hPSCs can comprise human embryonic stem cells (hESCs) and/or induced pluripotent stem cells (iPSCs). In one embodiment, the hPSCs can be autologous cells, although heterologous cells can also be used, such as when the patient being treated has received high-dose chemotherapy or radiation treatment to destroy the patient's immune system. In one embodiment, allogenic cells can be used. Where appropriate, the hPSCs can be obtained from a subject by means well-known in the art.

The CAR-expressing neutrophils can be loaded with any suitable nanoparticles, i.e., nanoparticles which can comprise a drug or prodrug, as known in the art. The nanoparticles can be loaded into the CAR-expressing neutrophils using any suitable method known in the art and/or exemplified herein.

In certain embodiments, the nanoparticles are biodegradable. In certain embodiments, the nanoparticles are biocompatible. The nanoparticles can comprise a biodegradable mesoporous organic silica nanoparticle. In certain embodiments, the nanoparticles comprise a biodegradable mesoporous organic silica nanoparticle with a rough surface (R—SiO₂). In certain embodiments, the nanoparticles comprise a biodegradable mesoporous organic silica nanoparticle with a smooth surface (S—SiO₂). Examples of suitable nanoparticles include, but are not limited to, rough silica nanoparticles, cytosine arabinoside-based liposomes e.g., DepoCyt®), polyamidoamine (PAMAM) dendrimer-albumin nanoparticles, and fullerene (e.g., gadofullerenol/fullerenol). The rough silica nanoparticles can be biodegradable mesoporous organic silica. In certain embodiments, the nanoparticles comprise one or more of rough silica nanoparticles, cytosine arabinoside-based liposomes, PAMAM dendrimer-albumin nanoparticles, and/or fullerene. In certain embodiments, the nanoparticles are biodegradable mesoporous organic silica.

The nanoparticles can comprise any drug (e.g., a therapeutic compound or agent) (e.g., as cargo therein) that can be used for therapeutic or prophylactic treatment, such as the therapeutic treatment of cancer. The drug can be a prodrug. The drug can be a preclinical or clinical drug or prodrug, an antineoplastic/chemotherapeutic drug, or a radiosensitizer.

Antineoplastic/chemotherapeutic drugs can be categorized as alkylating agents, antimetabolites, natural products, hormones and antagonists, and miscellaneous (see, e.g., Antineoplastic Agents, LiverTox: Clinical and Research Information on Drug-Induced Liver Injury [Internet publication], publicly available via the U.S. National Library of Medicine, National Center for Biotechnology Information website). Such drugs also can be classified by indication, mechanism of action, chemical structure, or cytotoxic/nonspecific vs. noncytotoxic/targeted. Examples of alkylating agents include, but are not limited to, altretamine, bendamustine, busulfan, carmustine, chlorambucil, cyclophosphamide, dacarbazine, ifosfamide, lomustine, mechlorethamine, melphalan, procarbazine, streptozocin, temozolomide, thiotepa, trabectedin, and platinum coordination complexes (e.g., carboplatin, cisplatin (a radiosensitizer), and oxaliplatin). Examples of antibiotics and cytotoxic agents include, but are not limited to, bleomycin, catinomycin, daunorubicin, doxorubicin, epirubicin, idarubicin, nutomycin (a radiosensitizer), mitoxantrone, plicamycin, and valrubicin. Nonlimiting examples of antimetabolites include antifolates (e.g., methotrexate, pemetrexed, pralatrexate, and trimetrexate), purine analogues (e.g., azathioprine, cladribine, fludarabine (a radiosensitizer), mercaptopurine, and thioguanine), and pyrimidine analogues (e.g., azacytidine, capecitabine, cytarabine, decitabine, floxuridine, fluorouracil (a radiosensitizer), gemcitabine (a radiosensitizer), and trifluridine/tipriacil). Biologic response modifiers include aldesleukin (IL-2), denileukin diftitox, and interferon gamma (IFNγ) as examples. Histone deactylase inhibitors include belinostat, Panobinostat, romidepsin, and vorinostat as examples. Hormonal agents include anti-androgens (e.g., abiraterone, apalutamide, bicalutamide, cyproterone, enzalutamide, flutamide, and nilutamide), anti-estrogens and aromatase inhibitors (e.g., anastrozole, exemestane, fulvestrant, letrozole, raloxifene, tamoxifen, and toremifene), gonadotropin releasing hormone analogues (e.g., degarelix, goserelin, histrelin, leuprolide, and triptorelin), and peptide honnones (e.g., lanreotide, octreotide, and pasireotide). Examples of monoclonal antibodies are numerous and include alemtuzumab, atezolizumab, bevacizumab, blinatumomab, cemiplimab, cetuximab, daratumumab, dinutuximab, elotuzumab, gemtuzumab, and inotuzumab among others.

Likewise, examples of protein kinase inhibitors are numerous and include abemaciclib, acalabrutinib, binimetinib, bortezomib, cabozantinib, carfilzomib, dabrafenib, dacomitinib, enasidenib, encorafenib, fedratinib, gefitinib, ibrutinib, lapatinib, midostaurin, and neratinib among others. Taxanes include, but are not limited to, cabazitaxel, docetaxel (a radiosensitizer), and paclitaxel (a radiosensitizer).

Topoisomerase inhibitors include, but are not limited to, etoposide, irinotecan teniposide (a radiosensitizer), and topotecan. Vinca alkaloids include, but are not limited to, vinblastine, vincristine, and vinorelbine. Other antineoplastic/chemotherapeutic agents include asparaginase, bexarotene, eribulin, everolimus, hydroxyurea (a radiosensitizer), ixabepilone, lenalidomide, mitotane, omacetaxine, pomalidomide, tagraxofusp, telotristate, temsirolimus, thalidomide, and venetoclax. In various embodiments, the drug is tirapazamine (a radiosensitizer), temozolomide, climacostol, or indole-3-acetic acid. In other embodiments, the drug is everolimus, bevacizumab, belzutifan, carmustine, naxitamab-gqgk, or lomustine. In certain embodiments, the drug is hypoxia-activated prodrug tirapazamine (TPZ) or the clinical chemotherapeutic drug temozolomide (TMZ). In certain embodiments, the drug is compound JNJ64619187.

In embodiments in which the nanoparticles comprise a prodrug, the prodrug can be activated by hypoxic conditions, acidic pH, an enzyme (e.g., horseradish peroxidase), irradiation, or the like.

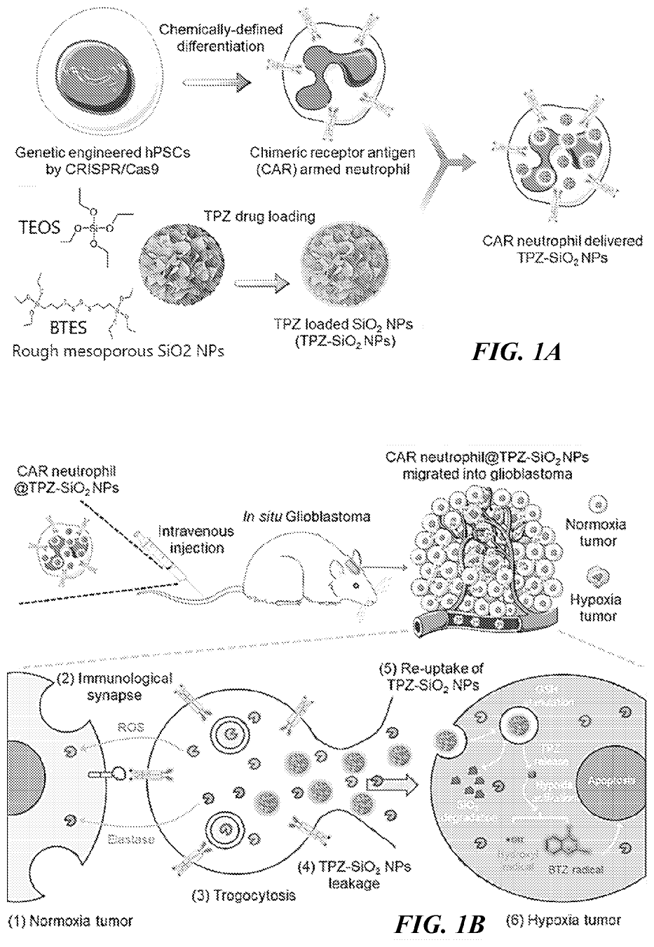

As described in more detail in the Examples, the hPSC-derived CAR-neutrophils hereof are unharmed by the nanoparticulated cargo and retain the inherent biophysiological properties of naïve neutrophils (FIG. 1A). For example, CAR-neutrophils loaded with TPZ- or TMZ-containing SiO₂ nanoparticles displayed superior anti-tumor activities against GBM by the combination of CAR-enhanced direct cytolysis and chemotherapeutic-mediated tumor killing upon intracellular release of TPZ or TMZ after cellular uptake and glutathione (GSH)-induced degradation of nanoparticles within the targeted tumor cells (FIG. 1B). In an in situ GBM xenograft model, hPSC-derived CAR-neutrophils specifically and effectively delivered TPZ-loaded SiO₂ nanoparticles to brain tumors without invasive surgical resection for amplified inflammation, significantly inhibited tumor growth, and prolonged animal survival. Anti-GBM activities were superior and specific, and off-target drug delivery was significantly reduced.

Still further, the CAR-neutrophil-mediated drug delivery system hereof can be solely dependent on the native chemo-attractant ability of GBM, without amplifying post-surgical inflammatory signals, which supports its high specificity and therapeutic potential in eradicating deeply infiltrated gliomas that cannot be removed by surgery. Since surgical resection and adjuvant chemotherapy/radiotherapy are the major clinical intervention for GBM, combination treatment with CAR-neutrophil nanocarrier and surgery/radiotherapy may achieve optimal therapeutic efficacy and is worth further investigation. Lin et al. (2021), supra.

The hPSC-neutrophil-based drug delivery platforms hereof can be modular and are versatile such that the CAR construct(s) and, ultimately, the CAR-expressing neutrophils can be re-engineered and tuned to support other neutrophil-based efforts in treating devastating human diseases. As noted above, CAR engineering is more accessible in hPSCs than primary immune T/NK cells, and it only requires one-time genome editing to achieve stable and homogenous expression of various CARs. In addition to the CLTX-CARs, stable hPSC lines have been constructed that express a universal anti-fluorescein (FITC) or anti-PD-L1 CAR, both of which can be harnessed to obtain universal solid tumor-targeting nanocarrier CAR-neutrophils. Lee et al., Regulation of CAR T cell-mediated cytokine release syndrome-like toxicity using low molecular weight adapters, Nature Communications 10: 2681 (2019); Kagoya et al., A novel chimeric antigen receptor containing a JAK-STAT signaling domain mediates superior antitumor effects, Nature Medicine 24: 352-359 (2018). Other genetic modifications, such as fibrosis targeting anti-FAP CARs, can also be performed to direct neutrophil nanocarriers to treat fatal regenerative diseases, including brain trauma and cardiac fibrosis. Aghajanian et al., Targeting cardiac fibrosis with engineered T cells, Nature 573: 430-433 (2019). Accordingly, in certain embodiments, the CAR constructs hereof comprise fibrosis targeting anti-FAP CAR constructs.

Furthermore, CAR-expressing hPSCs can also be easily adapted to produce CAR-T or NK cells, and combinations of these immunotherapies with CAR-neutrophil nanocarriers can have significant therapeutic anti-tumor benefits.

In summary, the CAR constructs and biomimetic CAR-expressing neutrophils (e.g., loaded with nanoparticles) provided herein provide a safe, potent and versatile platform for treating GBM and other devastating diseases. Also, given that neutrophils also preferentially phagocytose microbial pathogens with rough or long surfaces, such as S. aureus and E. coli, this attribute can be considered when designing nanoparticles for neutrophil-mediated drug delivery. Safari et al., Neutrophils preferentially phagocytose elongated particles—An opportunity for selective targeting in acute inflammatory diseases, Science Advances 6(24) (2020). Indeed, it has been recently reported that the preferred phagocytosis of intravenously administered elongated particles, without complicated surface modification, is by circulating neutrophils. Safari et al. (2020), supra; Chang et al., Crystallographic facet-dependent stress responses by polyhedral lead sulfide nanocrystals and the potential “safe-by-design” approach, Nano Research 9: 3812-3827 (2016); Chang et al., Achievement of safer palladium nanocrystals by enlargement of {100}crystallographic facets, Nanotoxicology 11(7): 907-922 (2017). Such an approach can maximize drug loading in neutrophils and enable therapeutic levels of drug delivery to be realized at targeted sites. Indeed, the findings presented herein support that treatments comprising administration of a combination of the functional CAR-neutrophils hereof and chemoimmunotherapy can exhibit superior and specific anti-GBM activities, significantly reduced off-target drug delivery, and prolonged lifespan in tumor-bearing mice.

Further, while the present disclosure illustrates the introduction of CAR into hPSCs followed by differentiation into neutrophils, it will be understood and appreciated by one of ordinary skill in the art that hPSC-derived hemogenic endothelial, hematopoietic progenitor cells, and neutrophils, such as those generated in accordance with a method described herein, can be directly targeted to make CAR-neutrophils.

Still further, the CAR-mediated drug delivery systems hereof are modular such that they are not limited in application to any particular disease or disorder, but instead, can be customized as desired. For example, the CAR neutrophils hereof can be prepared using any CARs desired (e.g., a CAR comprising a targeting-peptide with binding affinity for a disease- or disorder-specific target) and loaded with nanoparticles comprising a drug or prodrug selected to treat the targeted disease or disorder. This can be especially beneficial in view of the CAR neutrophils' ability to cross the blood brain barrier (BBB) and other biophysical barriers in a subject's body as such CAR neutrophils can deliver the nanoparticles to a site of interest within a subject irrespective of the presence of a BBB or other biophysical barrier.

### Engineered Neutrophil Cell Lines

Still further provided are engineered neutrophil cell lines (e.g., derived from hPSCs). In certain embodiments, the engineered neutrophil cell line comprises a CAR having/comprising a 36-amino acid GBM-targeting chlorotoxin peptide, a CD4, NKG2D, CD32a, or a CD16 transmembrane domain, and an intracellular domain (e.g., a CD3ζ intracellular signaling domain). The transmembrane domain can be a CD4 transmembrane domain. The transmembrane domain can be a NKG2D transmembrane domain. The transmembrane domain can be either a CD32a transmembrane domain or a CD16 transmembrane domain.

The intracellular domain can be a CD3ζ intracellular signaling domain. The intracellular domain can comprise a CD32aγ intracellular signaling domain or a CD16 intracellular signaling domain. In certain embodiments, the CAR further comprises a CD3ζ intracellular signaling domain (e.g., in addition to the other intracellular domain(s) of the CAR). The CAR can also further comprise a 2B4 co-stimulatory domain.

In certain embodiments, the transmembrane domain is a CD4 transmembrane domain, the intracellular domain is a CD3ζ intracellular signaling domain, and the CAR further has/comprises an IL-13Rα2-targeted quadruple mutant IL-13 (TQM13) T-CAR, GD2-targeting scFV, a HER2-targeting scFV, an EGFRvIII-targeting scFV, or other glioma-targeting scFV.

In certain embodiments, the CAR of the engineered neutrophil cell line comprises either a CD32a transmembrane domain or a CD16 transmembrane domain, and either a CD32aγ intracellular signaling domain or a CD16 intracellular signaling domain.

The engineered neutrophil cell line can comprise a CAR having/comprising a 36-amino acid GBM-targeting chlorotoxin peptide, a CD4 transmembrane domain, and a CD3ζ intracellular signaling domain. In certain embodiments, the engineered neutrophil cell line comprises a CAR having/comprising a 36-amino acid GBM-targeting chlorotoxin peptide, either a CD32a transmembrane domain or a CD16 transmembrane domain, and a CD3ζ intracellular signaling domain. In certain embodiments, the engineered neutrophil cell line comprises a CAR having/comprising a 36-amino acid GBM-targeting chlorotoxin peptide, either a CD32a transmembrane domain or a CD16 transmembrane domain, and either a CD32aγ intracellular signaling domain or a CD16 intracellular signaling domain. In certain embodiments, the engineered neutrophil cell line comprises a CAR having/comprising a 36-amino acid GBM-targeting chlorotoxin peptide, a NKG2D transmembrane domain, a 2B4 co-stimulatory domain, and a CD3ζ intracellular signaling domain. In certain embodiments, the engineered neutrophil cell line comprises a CAR having/comprising an IL-13 receptor α 2 (IL-13Rα2)-targeted quadruple mutant IL-13 (TQM13) T-CAR, GD2-targeting scFV, a HER2-targeting scFV, an EGFRvIII-targeting scFV, or other glioma-targeting scFV; a CD4 transmembrane domain; and a CD3ζ intracellular signaling domain. The engineered neutrophil cell line can comprise a CAR that additionally comprises a CD3ζ intracellular signaling domain.

### Compositions

Even still further provided is a pharmaceutical composition. The pharmaceutical composition can comprise a population of isolated CAR neutrophils described herein or otherwise obtained in accordance with a method hereof, or a population of neutrophils from an above-described cell line.

The pharmaceutical composition further comprises a pharmaceutically acceptable carrier and/or diluent.

The term “isolated” means that the material is removed from its original environment, e.g., the natural environment if it is naturally occurring. For example, a naturally occurring neutrophil present within a living organism is not isolated, but the same neutrophil separated from some or all the coexisting materials in the natural system is isolated.

The term “pharmaceutically acceptable” and grammatical variations thereof, as they refer to compositions, carriers, diluents, reagents, and the like, are used interchangeably and indicate that the materials can be administered to or upon a mammal without undue toxicity, irritation, allergic response, and/or the production of undesirable physiological effects, such as nausea, dizziness, gastric upset, and the like as is commensurate with a reasonable benefit/risk ratio. In other words, it is a material that is not biologically or otherwise undesirable—i.e., the material may be administered to an individual along with CAR neutrophils, for example, without causing any undesirable biological effects or interacting in a significantly deleterious manner with any of the other components of the pharmaceutical composition.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting a composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethanol; (20) phosphate buffered solutions; and (21) other non-toxic, compatible substances employed in pharmaceutical formulations.

The choice of carrier will be determined in part by the particular CAR, CAR-encoding nucleic acid sequence, vector, or host cells expressing the CAR, as well as by the particular method used to administer the CAR-encoding nucleic acid sequence, vector, or host cells expressing the CAR.

Accordingly, there are a variety of suitable formulations of the pharmaceutical composition. For example, the pharmaceutical composition can contain preservatives. Suitable preservatives may include, for example, methylparaben, propylparaben, sodium benzoate, and benzalkonium chloride. A mixture of two or more preservatives optionally can be used. The preservative or mixtures thereof are typically present in an amount of about 0.0001% to about 2% by weight of the total composition.

The particular formulation employed will also depend, at least in part, on the particular route of administration. For example, a formulation suitable for systemic, e.g.. intravenous, administration, may differ from a formulation suitable for intracranial administration. Such modifications are within the ordinary skill in the art.

### Methods of Treatment and Uses

A use of any of the CAR-expressing neutrophils provided hereof, any of the engineered neutrophil cell lines hereof or neutrophils derived therefrom, or pharmaceutical compositions hereof in the manufacture of a medicament for the treatment of a disease in a subject is provided. In certain embodiments, the disease is cancer. In certain embodiments, the disease is fibrosis.

A method of treating cancer in a subject (e.g., in need thereof) is also provided. The method can comprise administering to the subject a first therapy comprising a therapeutically effective amount of (a) a population of any of the CAR neutrophils described herein or a pharmaceutical composition comprising any of the CAR neutrophils hereof and a pharmaceutically acceptable carrier and/or diluent, or (b) a population of neutrophils from above-described cell line or a pharmaceutical composition comprising same and a pharmaceutically acceptable carrier and/or diluent.

In certain embodiments, the method can comprise a synergistic combination therapy, such that the method further comprises administering a second therapy to the subject (e.g., using a therapeutically effective amount) and provides an increased cytotoxic effect on a cancer in the subject as compared to administration to the subject of a single compound or therapy of the combination alone.

The second therapy can comprise surgical removal of one or more cancerous cells from the subject, chemotherapy, and/or radiotherapy (e.g., a therapeutically effective amount thereof). In certain embodiments, the method further comprises administering to the subject a therapeutically effective amount of chemotherapy to the subject. In certain embodiments, the method further comprises administering to the subject a therapeutically effective amount of radiotherapy to the subject. In certain embodiments, the method further comprises administering to the subject a therapeutically effective amount of both chemotherapy and radiotherapy to the subject.

In some embodiments, the cancer is additionally imaged prior to administration to the subject of the CAR neutrophils, or the CAR-expressing neutrophil composition. The cancer, additionally, or alternatively, can be imaged during or after administration to assess metastasis, for example, and the efficacy of treatment. In some embodiments, imaging occurs by positron emission tomography (PET) imaging, magnetic resonance imaging (MRI), or single-photon-emission computed tomography (SPECT)/computed tomography (CT) imaging. The imaging method can be any suitable imaging method known in the art.

In some embodiments, the method further comprises imaging the solid tumor cancer prior to or during administering of the CAR neutrophils, the composition comprising CAR neutrophils, and/or the second therapy.

The cancer can be any cancer. “Cancer” includes any neoplastic condition, whether malignant, pre-malignant or non-malignant, and includes a group of diseases involving abnormal cell growth with, in some cases, the potential to invade or spread (i.e., metastasize) to other parts of the body.

Generally, however, the neoplastic condition is malignant. Both solid and non-solid tumors are encompassed, and “cancer(ous) cell” may be used interchangeably with “tumor(ous) cell.”

Examples of cancers include, but are not limited to, leukemia (e.g., ALL, AML, CLL, and CML), adrenocortical carcinoma, AIDS-related cancer (e.g., Kaposi sarcoma), lymphoma (e.g., T-cell, Hodgkins, and non-Hodgkins), astrocytoma, basal cell carcinoma, bladder cancer, bone cancer, brain cancer (e.g., GBM), breast cancer, prostate cancer, lung cancer, cervical cancer, colon cancer, colorectal cancer, DCIS, esophageal cancer, gastric cancer, glioma, head and neck cancer, liver cancer, stomach cancer, pancreatic cancer, kidney cancer (e.g., renal cell and Wilms), oral cancer, oropharyngeal cancer, ovarian cancer, testicular cancer, and throat cancer.

The cancer can be a brain cancer. The brain cancer can be GBM. The cancer can be a prostate cancer. The cancer can express the protein matrix metallopeptidase 2. The population of neutrophils (e.g., CAR neutrophils and/or neutrophils from the engineered neutrophil cell line) or the pharmaceutical composition comprising the same can be administered systemically or intracranially.

Methods of delivering a therapeutic agent to a subject to a targeted location in a subject (e.g., with a disease) are also provided. In certain embodiments, the method comprises administering to the subject a first therapy comprising a therapeutically effective amount of a population of any of the CAR-expressing neutrophils hereof; a population of neutrophils from any of the engineered neutrophil cell lines hereof; or any of the pharmaceutical compositions hereof, wherein the targeted location is across a blood brain barrier of the subject relative to the site of administration.

The disease can be cancer (e.g., a brain cancer or a prostate cancer). The cancer can be any of the cancers described herein. The disease can be a glioblastoma. In embodiments where the disease is a cancer, the CARs of the CAR-expressing neutrophils used in the method can comprise a cancer-targeting peptide such as a chlorotoxin peptide, FAP, or other cancer-targeting peptides now known or hereinafter developed.

The disease can be a neurological disorder. The neurological disorder (e.g., a neurodegenerative disorder) can involve protein aggregation of proteins prone to aggregate. The neurological disorder can be a tauopathy. In certain embodiments where the disease is a neurological disorder, the CARs of the CAR-expressing neutrophils used in the method can comprise a brain-targeting (i.e., brain specific) peptide now known or hereinafter developed.

Proteins prone to aggregate include, but are not limited to, islet amyloid polypeptide, amyloid-β, α-synuclein (α-syn), tubulin associated unit (tau), and transthyretin. The tau can be tau isoform 2N4R or 1N4R. The CAR-expressing neutrophils described herein can be used to inhibit the aggregation of α-syn by being loaded with compound(s) for inhibiting such aggregation. Diseases involving protein aggregation include, but are not limited to, AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis.

The CAR-expressing neutrophils hereof can be used to inhibit the aggregation of α-syn in a subject having, or at risk for, Alzheimer's disease, dementia with Lewy bodies (DLB), or multiple system atrophy (MSA), especially due to the neutrophils' ability to cross the BBB and other biophysical barriers with a subject's body. The CAR-expressing neutrophils hereof can be used to inhibit the formation of α-syn inclusions in a subject with a neuroblastoma, for example, when loaded with a nanoparticle comprising a compound for inhibiting the formation of α-syn inclusions, In certain embodiments, the nanoparticles of the CAR-expressing neutrophils are selected from the group consisting of Donepezil, galantamine, aducanumab, and a monoclonal antibody (e.g., donanemab).

The CAR-expressing neutrophils hereof can be used to inhibit tau protein aggregation in tauopathies and the nanoparticles loaded therein can comprise any drug or prodrug appropriate for treating such a tauopathy. In certain embodiments, the drug or prodrug comprises a Fyn inhibitor (e.g., saracatinib), a GSK-3β inhibitor (e.g., tideglusib), or a p75 inhibitor (e.g., LM11A). Tauopathies are a group of disorders that result from abnormal tau phosphorylation, abnormal levels of tau, abnormal tau splicing, and mutations in the tau gene, for example. Neurodegenerative diseases have been classified based on this protein accumulation. Tauopathies encompass more than 20 clinicopathological conditions, including Alzheimer's disease (AD), which is the most common tauopathy. Other tauopathies include, but are not limited to, familial AD, primary age-related tauopathy (PART), Creutzfeldt-Jacob disease, dementia pugilistica, Gerstmann-Straussler-Scheinker disease (GSS), inclusion-body myositis, cortico-basal degeneration (CBD), Picks disease (PiD), progressive supranuclear palsy (also known as Steele, Richardson, and Olszewski disorder), Down syndrome, Parkinsonism with dementia, myotonic dystrophy, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, diffuse neurofibrillary tangles with calcification, frontotemporal dementia with Parkinsonism linked to chromosome 17 (FTDP-17), Haller-vorden-Spatz disease, multiple system atrophy (MSA), Niemann-Pick disease type C, pallido-ponto-nigral degeneration, progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD, Huntington's disease, and prion diseases with tangles. The majority of neurodegenerative diseases are characterized by the deposition of insoluble protein in cells of the neuromuscular system.

Accordingly in certain embodiments of the method for delivering a therapeutic agent to a subject with a disease, the disease is Alzheimer's disease. In certain embodiments of the method for delivering a therapeutic agent to a subject with a disease, the disease is Parkinson's disease.

The method of delivering a therapeutic agent to a subject with a disease can further comprise administering a second therapy to the subject. The second therapy can be, for example, surgical removal of cancerous cells from the subject (e.g., where the disease is cancer). The second therapy can comprise a chemotherapy, radiotherapy, or both. The second therapy can comprise, for example where the disease is cancer, imaging a targeted location (e.g., a cancer (e.g., a TME) or a brain tissue) in the subject prior to or during administering the first and/or second therapies. In certain embodiments, for example, where the disease is a neurological disorder, the second therapy can comprise a microtubule-stabilizing agent such as, without limitation, docetaxel, epothilone D. and/or paclitaxel. In certain embodiments, the first and second therapies are administered sequentially and/or alternatively relative to each other.

The methods hereof can reduce, even substantially reduce, systemic and off-target toxicity. “Off-target toxicity” means organ or tissue damage or a reduction in the subject's weight that is not desirable to the physician or other individual treating the subject, or any other effect on the subject that is a potential adverse indicator to the treating physician (e.g., B cell aplasia, a fever, a drop in blood pressure, or pulmonary edema).

The terms “treat,” “treating,” “treated,” and “treatment” (with respect to a disease or condition, such as cancer) are used to describe a method for obtaining beneficial or desired results, such as clinical results, which can include, but are not limited to, one or more of improving a condition associated with a disease, curing a disease, lessening severity of a disease, increasing the quality of life of one suffering from a disease, prolonging survival and/or a prophylactic treatment. In reference to cancer, in particular, the terms “treat,” “treating,” “treated,” or “treatment” can additionally mean reducing the size of a tumor, completely or partially removing the tumor (e.g., a complete or partial response), stabilizing a disease, preventing progression of the cancer (e.g., progression-free survival), or any other effect on the cancer that would be considered by a physician to be a therapeutic or prophylactic treatment of the cancer. More particularly, curative treatment refers to any of the alleviation, amelioration and/or elimination, reduction and/or stabilization (e.g., failure to progress to more advanced stages) of a sign/symptom, as well as delay in progression of a sign/symptom of a particular disorder. Prophylactic treatment refers to any of the following: halting the onset, reducing the risk of development, reducing the incidence, delaying the onset, reducing the development, and increasing the time to onset of symptoms of a particular disorder. Desirable effects of treatment can include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions are used to delay development of a disease and/or tumor, or to slow (or even halt) the progression of a disease and/or tumor growth.

The term “patient” or “subject” includes human and non-human animals, such as companion animals (dogs and cats and the like) and livestock animals. Livestock animals are animals raised for food production. The subject to be treated is preferably a mammal, in particular a human being.

As used herein, the term “administering” includes all means of introducing the neutrophils and pharmaceutical compositions comprising same, to the patient. Examples include, but are not limited to, oral (po), parenteral, systemic/intravenous (iv), intramuscular (im), subcutaneous (sc), transdermal, intrasternal, intraarterial, intraperitoneal, epidural, intraurethral, intranasal, buccal, ocular, sublingual, vaginal, rectal, and the like. Routes of administration to the brain include, but are not limited to, intraparenchymal, intraventricular, intracranial, and the like.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients, such as salts, carbohydrates and buffering agents (preferably at a pH in the range from about 3 to about 9). The preparation of parenteral formulations under sterile conditions may readily be accomplished using standard pharmaceutical techniques well-known to those skilled in the art.

The neutrophils can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, the neutrophils, or composition comprising the same, can be administered directly into the blood stream, into muscle, or into an internal organ.

The neutrophils/compositions can be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9).

The percentage of the neutrophils, compositions and preparations may vary and may be between about 1 to about 99% weight of the active ingredient(s) and a binder, excipients, a disintegrating agent, a lubricant, and/or a sweetening agent (as are known in the art). The amount of the neutrophils in such therapeutically useful compositions is such that an effective dosage level will be obtained.

In some embodiments, the neutrophils are administered as a composition comprising one or more pharmaceutically acceptable carriers, adjuvants, diluents, excipients, vehicles, or a combination of any of the foregoing.

The term “therapeutically effective amount” as used herein, refers to that amount of engineered neutrophils that elicits the biological or medicinal response in a tissue system, animal or human that is being sought by a researcher, veterinarian, medical doctor or other clinician (e.g., a desired therapeutic effect), which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the engineered neutrophils may be decided by the attending physician within the scope of sound medical judgment. In the treatment of cancer, a desired therapeutic effect can range from inhibiting the progression of cancer, e.g., proliferation of cancerous cells and/or the metastasis thereof. Desirably, the administration of a therapeutically sufficient amount kills cancerous cells, such that the number of cancerous cells decreases, desirably to the point of eradication.

The specific therapeutically effective dose level of CAR neutrophils for any particular patient will depend upon a variety of factors, including the disorder being treated and the state/severity of the disorder; the specific composition employed; the age, body weight, general health, gender and diet of the patient; the time and route of administration; the duration of the treatment; drugs used in combination or coincidentally with the engineered neutrophils; and like factors well-known to the researcher, veterinarian, medical doctor or other clinician of ordinary skill. By way of example, a dose of CAR-expressing neutrophils may range from 10⁵ to 10¹² per m² of the patient's body surface area or per kg of the patient's weight. Thus, the absolute amount of engineered neutrophils included in a given unit dosage form can vary widely, and depends upon factors such as the age, weight and physical condition of the subject, as well as the method of administration.

Depending upon the route of administration, a wide range of permissible dosages are contemplated herein. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

Multiple infusions may be required in order to treat a subject effectively. For example, 2, 3, 4, 5, 6 or more separate infusions may be administered to a patient at intervals of from about 24 hours to about 48 hours, or every 3, 4, 5, 6, or 7 days. Infusions may be administered weekly, biweekly, or monthly. Monthly administrations can be repeated from 2-6 months or longer, such as 9 months to year.

Administered dosages for the engineered neutrophils for treating cancer, such as a brain tumor, a glioma, a GBM, or a cancer expressing MMP2, or other disease or disorder are in accordance with dosages and scheduling regimens practiced by those of skill in the art. Typically, doses >10⁹ cells/patient are administered to patients receiving adoptive cell transfer therapy. Determining an effective amount or dose is well within the capability of those skilled in the art, especially in light of the detailed disclosure provided herein.

The engineered neutrophils administered to a subject can comprise about 1×10⁵ to about 1×10¹⁵ or 1×10⁶ to about 1×10¹⁵ transduced CAR-T cells. In various embodiments about 1×10⁵ to about 1×10¹⁰, about 1×10⁶ to about 1×10¹⁰, about 1×10⁶ to about 1×10⁹, about 1×10⁶ to about 1×10⁸, about 1×10⁶ to about 2×10⁷, about 1×10⁶ to about 3×10⁷, about 1×10⁶ to about 1.5×10⁷, about 1×10⁶ to about 1×10⁷, about 1×10⁶ to about 9×10⁶, about 1×10⁶ to about 8×10⁶, about 1×10⁶ to about 7×10⁶, about 1×10⁶ to about 6×10⁶, about 1×10⁶ to about 5×10⁶, about 1×10⁶ to about 4×10⁶, about 1×10⁶ to about 3×10⁶, about 1×10⁶ to about 2×10⁶, about 2×10⁶ to about 6×10⁶, about 2×10⁶ to about 5×10⁶, about 3×10⁶ to about 6×10⁶, about 4×10⁶ to about 6×10⁶, about 4×10⁶ to about 1×10⁷, about 1×10⁶ to about 1×10⁷, about 1×10⁶ to about 1.5×10⁷, about 1×10⁶ to about 2×10⁷, about 0.2×10⁶ to about 1×10⁷, about 0.2×10⁶ to about 1.5×10⁷, about 0.2×10⁶ to about 2×10⁷, or about 5×10⁶ cells.

The engineered neutrophils administered to a subject can comprise about 1 million, about 2 million, about 3 million, about 4 million, about 5 million, about 6 million, about 7 million, about 8 million, about 9 million, about 10 million, about 11 million, about 12 million, about 12.5 million, about 13 million, about 14 million, or about 15 million cells. The cells can be administered as a single dose or multiple doses. The engineered neutrophils can be administered in numbers of CAR-expressing neutrophils per kg of subject body weight.

The CAR-expressing neutrophils can be administered by any suitable route. Such routes include, but are not limited to, intravenous and intratumoral. The formulation of compositions suitable for administration of CAR-expressing neutrophils, including compositions suitable for administration by intravenous and intratumoral routes, is within the ordinary skill in the art. The CAR-expressing neutrophils composition can comprise one or more pharmaceutically acceptable carriers, diluents, and/or other pharmaceutically acceptable components. The carriers, diluents, and/or other components can be determined in part by the particular route of administration (see, e.g., Remington's Pharmaceutical Sciences, 17^{th} ed. (1985)). The ingredients of the composition must be of sufficiently high purity and sufficiently low toxicity such that the composition is suitable for administration to a human. The composition desirably is stable.

### General

All patents, patent application publications, journal articles, textbooks, and other publications mentioned in the specification are indicative of the level of skill of those in the art to which the disclosure pertains.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note.

Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about,” when referring to a number or a numerical value or range (including, for example, whole numbers, fractions, and percentages), means that the number or numerical range referred to is an approximation within experimental variability (or within statistical experimental error), and thus the numerical value or range can vary between 1% and 15% of the stated number or numerical range (e.g., +/−5% to 15% of the recited value), provided that one of ordinary skill in the art would consider equivalent to the recited value (e.g., having the same function or result).

When ranges are used herein for physical properties, such as molecular weight, or chemical properties, such as chemical formulae, all combinations and sub-combinations of ranges and specific embodiments therein are intended to be included.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise. the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

The term “receptor” refers to a chemical structure in biological systems that receives and transmits signals.

Unless otherwise expressly stated, depicted structures include all stereochemical forms of the structure, i.e., the right-hand (R) and left-hand (S) configurations of each asymmetric center. Therefore, single stereochemical isomers, as well as enantiomeric and diastereomeric mixtures, are within the scope of the present disclosure.

One of ordinary skill in the art will further appreciate that the above SMDCs can be “deuterated,” meaning one or more hydrogen atoms can be replaced with deuterium. As deuterium and hydrogen have nearly the same physical properties, deuterium substitution is the smallest structural change that can be made. Replacement of hydrogen with deuterium can increase stability in the presence of other drugs, thereby reducing unwanted drug-drug interactions, and can significantly lower the rate of metabolism (due to the kinetic isotope effect). By lowering the rate of metabolism, half-life can be increased, toxic metabolite formation can be reduced, and the dosage amount and/or frequency can be decreased.

Pharmaceutical compositions are also provided. As used herein, the term “composition” generally refers to any product comprising more than one ingredient, e.g., one or more populations of CAR neutrophils and a carrier.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

While the present disclosure has been made with reference to humans and human cells and genes, it is contemplated that CAR-expressing neutrophils can be generated from other species, such as other species of mammals, using cells and genes from that species. Such CAR-expressing neutrophils then can be used to treat members of that species in accordance with the teachings provided herein.

### EXAMPLES

The following examples serve to illustrate the present disclosure. The examples are not intended to limit the scope of the claimed invention in any way.

### Materials and Methods

General methodologies and materials used in the Examples described herein are provided in this section. Any study-specific modifications to these general materials and methods will be set forth in the particular Example.

TABLE 1
Antibodies used in these Examples

| Antibody | SOURCE | SOURCE |
|---|---|---|
| CD45-PE | BD Biosciences | Cat#555483 |
| CD45-APC | BD Biosciences | Cat#555485 |
| CD45-FITC | BioLegend | Cat#304006 |
| CD66b-PE | BD Biosciences | Cat#561650 |
| MPO-FITC | Invitrogen | Cat#11129941 |
| CD11b-APC | BD Biosciences | Cat#561015 |
| CD16-FITC | BD Biosciences | Cat#556618 |
| CD18-FITC | Miltenyi Biotec | Cat#130120322 |
| CD15-PE | BD Biosciences | Cat#562371 |
| Actin-stain ™ | Cytoskeleton | Cat#PHDG1 |
| Syk Rb mAb | Cell Signaling | Cat#13198T |
| p-Syk Rb mAb | Cell Signaling | Cat#2710T |
| MAPK Rb mAb | Cell Signaling | Cat#4695T |
| p-MAPK Rb mAb | Cell Signaling | Cat#4370T |
| SSEA-4 Ms IgG | Santa Cruz | Cat#sc-21704 |
| OCT-3/4 Ms IgG | Santa Cruz | Cat#sc-5279 |
| Anti-Rb IgG HRP | Cell Signaling | Cat#7074S |
| β-actin Rb mAb | Cell Signaling | Cat#5125S |
| Alexa 488 Goat anti-Ms | Thermo Fisher Scientific | Cat#A-21121 |
| Alexa 488 Goat anti-Rb IgG | Thermo Fisher Scientific | Cat# A-11008 |
| Alexa 594 Goat anti-Ms IgG | Thermo Fisher Scientific | Cat# A-21145 |
| Alexa 594 Goat anti-Ms IgG | Thermo Fisher Scientific | Cat#A-11012 |
| Alexa 647 Goat anti-Rb IgG | Thermo Fisher Scientific | Cat# A-21244 |

Synthesis of degradable dendritic mesoporous organosilica nanoparticles (DDMONs). DDMONs were synthesized via a one-pot synthesis using NaSal and cationic surfactant cetyltrimethylammonium bromide (CTAB) as structure directing agents, tetraethyl orthosilicate (TEOS) and bis[3-(triethoxysilyl)propyl]tetrasulfide (BTES) as silica source, and triethanolamine (TEA) as a catalyst. The synthesis was conducted in a 50 mL flat bottom glass bottle with a 3-cm stirring bar. Typically, 0.034 g of triethylamine (TEA) was added to 12.5 mL of water and stirred gently (˜700 rpm) at 80° C. in an oil bath under a magnetic stirrer for 0.5 hours. Afterward, 190 mg of CTAB and 42 mg of NaSal were added to the above solution, stirring for another 1 hour. After CTAB and NaSal were completely dissolved, a mixture of 1 mL of TEOS and 0.8 mL of BTES was added to the mixture solution, followed by vigorous stirring for 12 hours. The nanoparticles were collected by centrifugation at 20,000 rpm for 5 minutes and washed three times with ethanol to remove residual reactants. The powder was then dried in a vacuum oven at 40° C. for 6 hours.

Collected products were extracted with HCl and methanol solution at 60° C. for 6 hours three times to remove the template, followed by overnight vacuum drying at room temperature.

Preparation of sphere mesoporous silica nanoparticles (SMSNs). To prepare the SMSNs, 240 mL of aqueous solution containing 0.5 g of CTAB were prepared in a conical flask. 1.5 mL of NaOH (2 mol L⁻¹) were then added to the CTAB solution with stirring for 10 minutes. Once the mixture temperature was adjusted to 80° C., 2.5 mL of TEOS were added dropwise to the solution and stirred for 2 hours. The resulting SMSNs were then centrifuged and washed with ethanol and deionized water several times to remove surfactant templates. The remaining powder was dried in a vacuum oven at 40° C. for 6 hours. The collected products were extracted with HCl and methanol solution at 60° C. for 6 hours three times to remove residual template, followed by overnight vacuum drying at room temperature.

Flow cytometry analysis. Cell cultures were gently pipetted and filtered through a 70 or 100 μm strainer sitting on a 50 mL tube. The cells were then pelleted by centrifugation and washed twice with PBS−/− solution containing 1% bovine serum albumin (BSA). The cells were stained with appropriate conjugated antibodies for 25 minutes at room temperature in the dark and analyzed in an Accuri C6 plus cytometer (Beckton Dickinson) after washing with BSA-containing PBS−/− solution. FlowJo software was used for flow data analysis.

Measurement of reactive oxygen species (ROS) production. U87MG cells (100 μL; 30,000 cells/mL) were seeded into wells of a 96-well plate 12 hours before adding neutrophils at a neutrophil-to-tumor ratio of 10:1. After co-incubation for 12 hours, the resulting cell mixture was treated with 10 μM H₂DCFDA at 37° C. for 50 minutes, and the fluorescence emission signal (480-600 nm) was collected in a SpectraMax iD3 microplate reader (Molecular Devices, Sunnyvale, CA) with an excitation wavelength of 475 nm.

Statistical analysis. Data are presented as mean f standard deviation (SD). Statistical significance was determined by Student's t-test (two-tail) between two groups, and three or more groups were analyzed by one-way analysis of variance (ANOVA). P<0.05 was considered statistically significant.

### Example 1

Screening CAR Structures with Enhanced Neutrophil-Mediated Anti-Tumor Activities

Four different CAR structures were first designed, screened, and optimized for anti-tumor activities of hPSC-neutrophils. The donor plasmids targeting AAVS1 locus were constructed as previously described in Chang et al., Fluorescent indicators for continuous and lineage-specific reporting of cell-cycle phases in human pluripotent stem cells, Biotechnology & Bioengineering 117(7): 2177-2186 (2020). Briefly, a chlorotoxin (CLTX) sequence containing a granulocyte-macrophage colony-stimulating factor receptor (GM-CSFR) signal peptide and IgG4 hinge, and CD3ζ and/or CD32aγ with CD4 or CD32a transmembrane domain were directly synthesized (GeneWiz) and cloned into the AAVS1-Puro CAG-FUCCI donor plasmid (Addgene #136934). Wang et al. (2020), supra. The resulting CLTX CAR constructs were sequenced and submitted to Addgene (#171963 to #171865; SEQ ID NOS. 1-3).

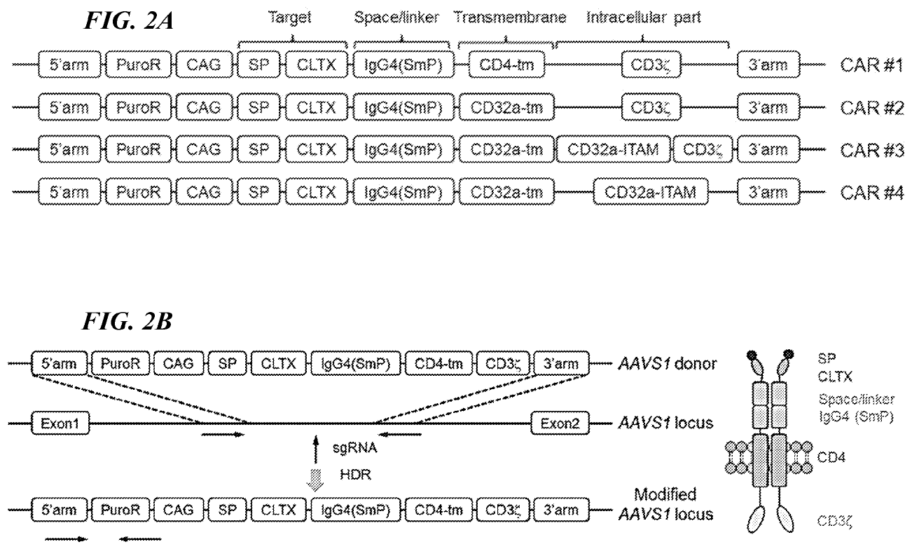

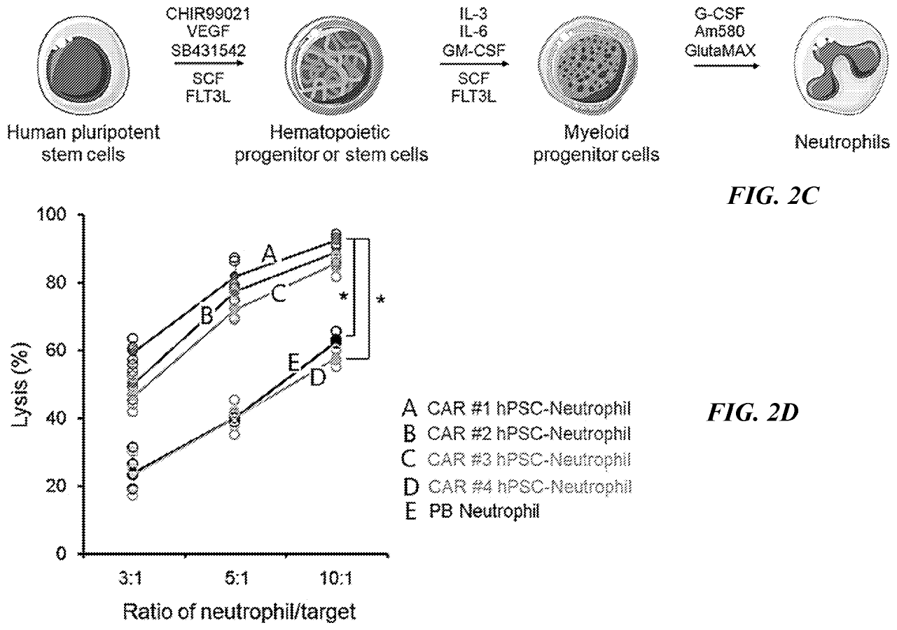

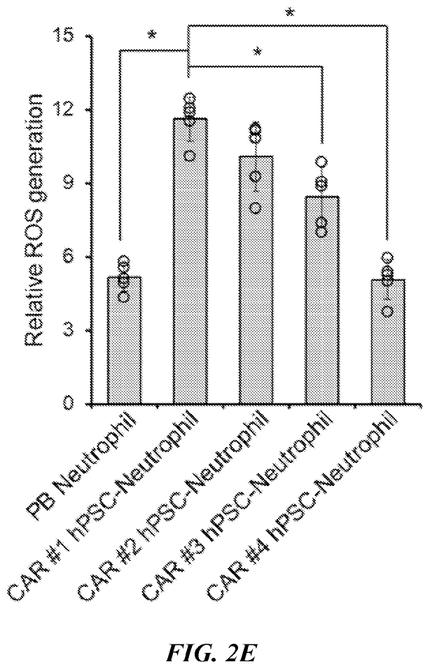

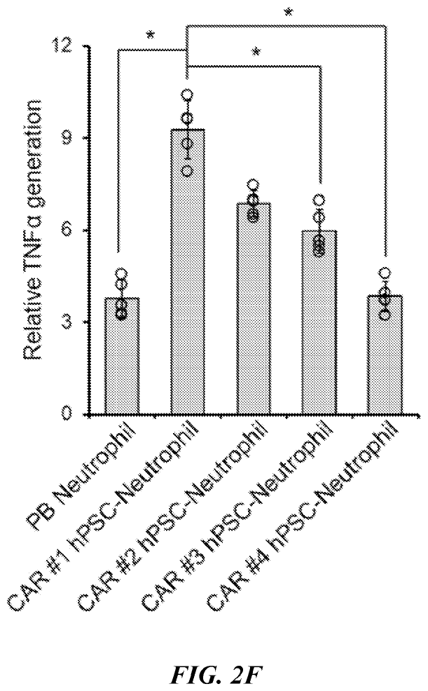

As noted above, all four of these CAR structures shared the same extracellular granulocyte-macrophage colony-stimulating factor receptor (GM-CSFR) signal peptide (SP), glioblastoma-targeting domain CLTX and IgG4 hinge (FIG. 2A). Wang et al. (2020), supra; Chang et al. (2022), supra.

CAR #1 (SEQ ID NO: 1) was a first generation of T cell-specific CAR that used a CD4 transmembrane (tm) domain and CD3ζ intracellular signaling domain. CAR #2 (SEQ ID NO: 2), CAR #3 (SEQ ID NO: 3), and CAR #4 (SEQ ID NO: 4) differed from CAR #1 in using a transmembrane domain from neutrophil-specific CD32a (or FcγRIIA), a single-chain transmembrane receptor that highly expresses in neutrophils (30,000 to 60,000 molecules/cell) and is critical for neutrophil activation. Wang & Jonsson (2019), supra; Nemeth et al., Importance of fc receptor γ-chain ITAM Tyrosines in neutrophil activation and in vivo autoimmune arthritis, Frontiers in Immunology 10 (2019); Role of neutrophil FcγRIIa (CD32) and FcγRIIIb (CD16) polymorphic forms in phagocytosis of human IgG1- and IgG3-opsonized bacteria and erythrocytes, Transfusion Medicine Reviews 9(4): 343 (1995); Tsuboi et al., Human Neutrophil Fcγ Receptors Initiate and Play Specialized Nonredundant Roles in Antibody-Mediated Inflammatory Diseases, Immunity 28(6): 833-846 (2008).

CAR #3 and CAR #4 also included a Fc domain γ-chain of CD32a, which relies on a highly conserved immunoreceptor tyrosine-based activation motif (ITAM) to express and signal in neutrophils. Notably, CAR #3 also contained a combo signaling domain by fusing CD32a-ITAM to the CD3ζ intracellular domain.

Since primary neutrophils are short-lived and resistant to genome editing, hPSCs were engineered with these CARs to achieve stable and universal immune receptor expression on differentiated neutrophils by knocking CAR constructs into the AAVS1 safe harbor locus via CRISPR/Cas9-mediated homology-directed repair (FIG. 2B). Briefly, a H₉ hPSC line was obtained from WiCell and maintained on Matrigel-coated plates in mTeSR plus medium. For neutrophil differentiation, hPSCs were dissociated with 0.5 mM ethylenediaminetetraacetic acid (EDTA) and seeded onto iMatrix 511-coated 24-well plates at a cell density between 10,000 and 80,000 cells/cm² in mTeSR plus medium with 5 μM Y27632 for 24 hours (day-1).

At day 0, cells were treated with 6 NM CHIR99021 (CHIR) in Dulbecco's Modified Eagle Medium (DMEM) supplemented with 100 μg/mL ascorbic acid (DMEM/Vc), followed by a medium change with LaSR basal medium from day 1 to day 4. Vascular endothelial growth factor (VEGF) (50 ng/mL) was added to the medium from day 2 to day 4. At day 4, the medium was replaced by Stemline II (Sigma) supplemented with 10 M SB431542, 25 ng/mL SCF and FLT3L. On day 6, SB431542-containing medium was aspirated, and the cells were maintained in Stemline II medium with 50 ng/mL SCF and FLT3L.

At days 9 and 12, the top half medium was aspirated and changed with 0.5 ml fresh Stemline II containing 50 ng/mL SCF, 50 ng/mL FLT3L, and 25 ng/mL GM-CSF. On day 15 floating cells were gently harvested and filtered for terminal neutrophil differentiation in Stemline II supplemented with 1× GlutaMAX, 150 ng/mL G-CSF, and 2.5 M retinoic acid agonist AM580. A half medium change was performed every 3 days, and mature neutrophils were harvested from day 21.

To increase cell viability, 10 μM Y27632 was used to treat hPSCs for 3-4 hours or overnight before nucleofection. Cells were then singularized by Accutase for 8-10 minutes, and 1-2.5×10⁶ hPSCs were nucleofected with 6 μg SpCas9 AAVS1 gRNA T2 (Addgene #79888) and 6 g CAR donor plasmids in 100 μl human stem cell nucleofection solution (Lonza #VAPH-5012) using program B-016 in a Nucleofector 2b.

The nucleofected cells were seeded into one well of a Matrigel-coated 6-well plate with 3 nil pre-warmed mTeSR plus and 10 μM Y27632. 24 hours later, the medium was changed with fresh mTeSR plus containing 5 μM Y27632, followed by daily medium change. When cells were more than 80% confluent, drug selection was performed with 1 g/ml puromycin (Puro) for about 1 week until nickel-sized hPSC clones were visible. Individual clones were then picked and expanded for 2-5 days in each well of a 96-well plate pre-coated with Matrigel, followed by a PCR genotyping. The genomic DNA of single clone-derived hPSCs was extracted with QuickExtract™ DNA Extraction Solution (Epicentre #QE09050). 2×GoTaq Green Master Mix (Promega #7123) was used to perform genomic DNA PCR. For positive genotyping, the following primer pair with an annealing temperature Tm of 65° C. was used: CTGTTTCCCCTTCCCAGGCAGGTCC (SEQ ID NO: 5) and TCGTCGCGGGTGGCGAGGCGCACCG (SEQ ID NO: 6). For homozygous screening, we used the following set of primer sequences: CGGTTAATGTGGCTCTGGTT (SEQ ID NO: 7) and GAGAGAGATGGCTCCAGGAA (SEQ ID NO: 8) with an annealing temperature Tm of 60° C.

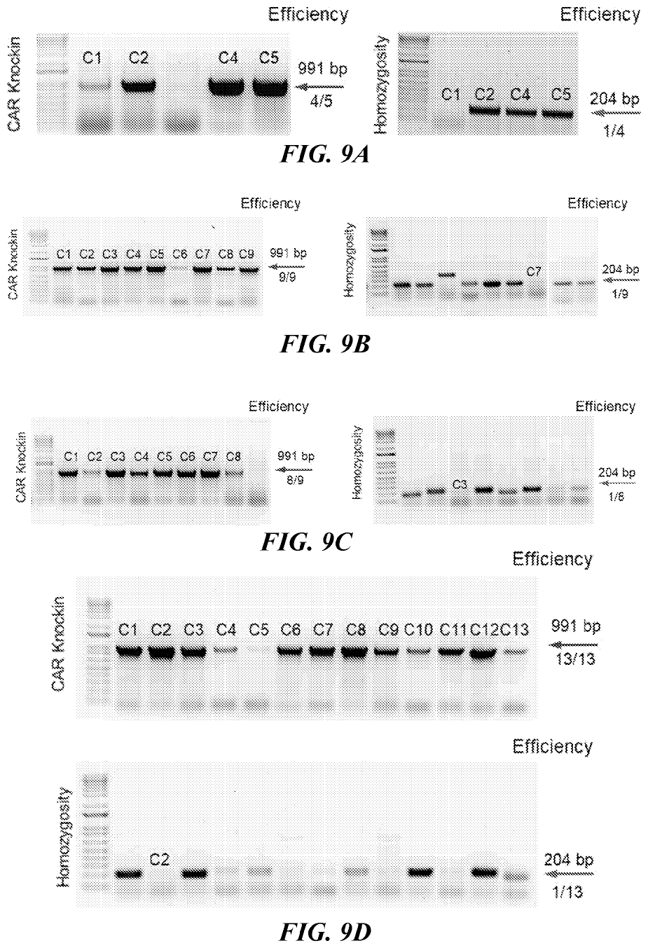

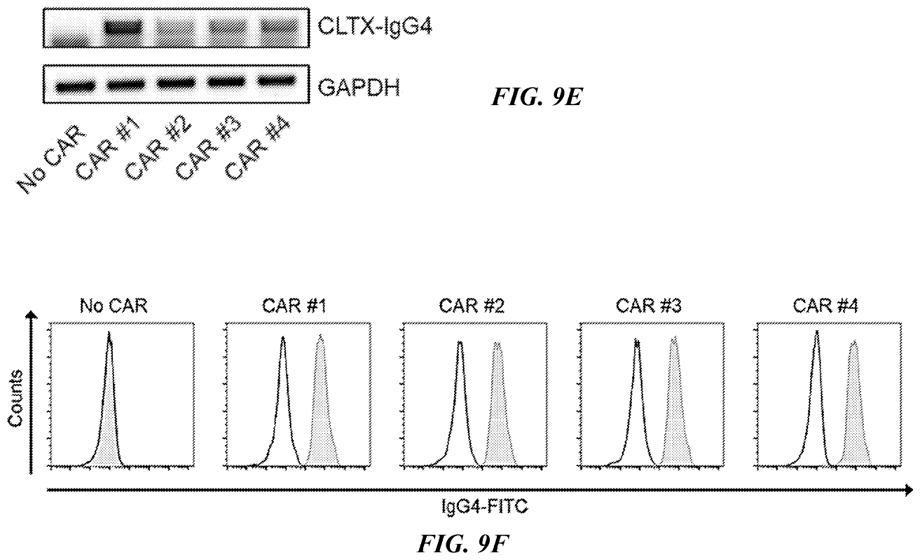

After nucleofection, single cell-derived hPSC clones were isolated and screened with puromycin for about 2 weeks. Genotyping identified successfully targeted hPSCs with an average CAR knock-in efficiency of >90%, and the majority of the targeted clones were heterozygous (FIGS. 9A-9D). CAR expression on engineered hPSCs was further confirmed by RT-PCR and flow cytometry analysis of CLTX-IgG4 (performed pursuant to the methodologies described above) (FIGS. 9E and 9F).

To produce de novo CAR-neutrophils, CAR-expressing hPSCs were first differentiated into multipotent hematopoietic and then myeloid progenitors with stage-specific cytokine treatment (FIG. 2C). Chang et al., Chemically-defined generation of human hemogenic endothelium and definitive hematopoietic progenitor cells, Biomaterials 285: 121569 (2022). Subsequent employment of G-CSF and retinoic acid agonist AM580 promoted robust neutrophil production. Brok-Volchanskaya et al., Effective and Rapid Generation of Functional Neutrophils from Induced Pluripotent Stem Cells Using ETV2-Modified mRNA, Stem Cell Reports 13(6): 1099-1110 (2019).

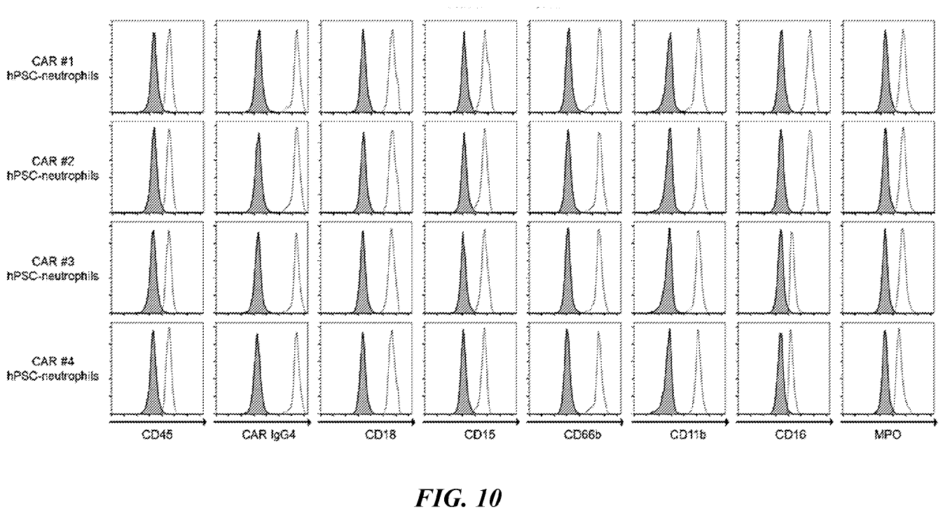

Similar to their counterparts in peripheral blood (PB), hPSC-derived CLTX-CAR neutrophils presented typical neutrophil morphology and surface markers CD16, CD11b, MPO, CD15, CD66b, and CD18 (FIG. 10).

The effects of CAR expression on the anti-tumor cytotoxicity of hPSC-derived neutrophils was determined by co-culturing them with glioblastoma (GBM) U87MG cells in vitro and cell viability was analyzed by flow cytometry. Briefly, 100 μL of GBM U87MG cells tumor cells (50,000 cells/mL) were mixed with 100 L of 150,000, 250,000 and 500,000 cells/mL the CAR neutrophils in 96 well plates, and then incubated at 37° C., 5% CO2 for 24 hours. To harvest all the cells, cell-containing media was first transferred into a new round-bottom 96-well plate, and 50 L of trypsin-EDTA were added to the empty wells. After a 5-minute incubation, attached cells were dissociated and transferred into the same wells of round-bottom 96-well plate with suspension cultures. All cells were pelleted by centrifuging the 96-well plate at 300×g, 4° C. for 4 minutes, and washed with 200 μL of PBS−/− solution containing 0.5% BSA.

For the cytotoxicity analysis, both live/dead cell staining and the CytoTox-Glo™ Cytotoxicity Assay kit (Promega, Madison, WI) were employed. CytoTox-Glo™ Cytotoxicity analysis and quantification were determined by SpectraMax iD3 microplate reader (Molecular Devices, LLC, Sunnyvale, CA).

As expected, hPSC-derived CLTX-CAR neutrophils presented improved tumor-killing ability as compared to PB neutrophils (FIG. 2D), consistent with previous observation in CLTX CAR-T cells. Wang et al. (2020), supra.

Among these different CARs, CAR #1 mediated superior tumor-killing activities in hPSC-neutrophils. Notably, γ-chain-based CAR #4 was the least effective in triggering neutrophil-mediated tumor-killing, which could be due to the lower copy of ITAM in γ than ζ-subunit and lower expression of γ-bearing CAR on the cell surface. Roberts et al. (1998), supra. Neutrophils could also release cytotoxic reactive oxygen species (ROS) and tumor necrosis factor-α (TNF-α) to kill target cells, and production of ROS and TNF-α (FIGS. 2E and 2F) in different neutrophils coincided well with their increased cytolysis. indicating the involvement of ROS and inflammation cytokines in neutrophil-mediated cytotoxicity against GBM cells.

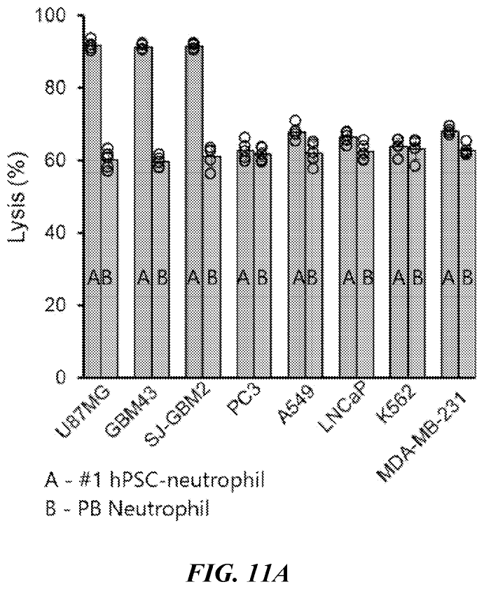

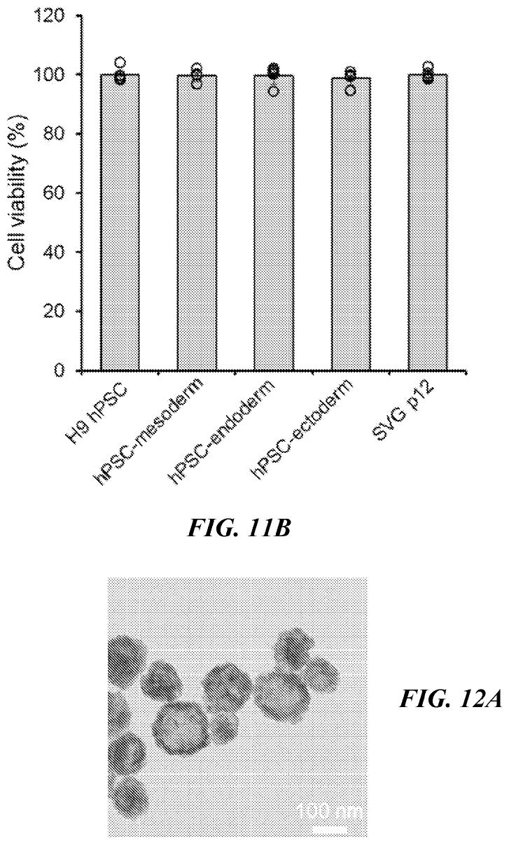

In addition, enhanced anti-tumor cytotoxicity of CAR-neutrophils was only observed in co-incubation with GBM cells, including U87MG, primary adult GBM43, and pediatric SJ-GBM2 cells (FIG. 11A), demonstrating the high specificity of the CLTX-CAR. Notably, CAR-neutrophils exhibited high biocompatibility on normal SVG p12 glial cells, hPSCs or hPSC-derived cells (FIG. 11B), consistent with a previous observation that primary neutrophils do not kill healthy cells. Yan et al. (2014), supra. Collectively, hPSC-derived CAR-neutrophils, particularly CD3ζ-bearing CAR-neutrophils, presented enhanced anti-tumor cytotoxicity and produced more ROS and TNF-α in vitro, highlighting their potential in targeted immunotherapy.

### Example 2

### CAR-Neutrophils Sustained Superior Anti-Tumor Activities Under Immunosuppressive Tumor Microenvironment

Similar to macrophages, anti-tumor N1 and pro-tumor N2 phenotypes of tumor-associated neutrophils were found within the immunosuppressive tumor microenvironment (TME). Jaillon et al. (2020), supra. Pro-tumor N2 neutrophils play critical roles in tumor angiogenesis, metastasis and immunosuppression, but therapeutic targeting of this cell type has been challenging. Unlike the systematic depletion strategy, the potential of CAR-engineering in sustaining anti-tumor phenotype of neutrophils was evaluated. Yee et al. (2020), supra.

CAR hPSCs and PB neutrophils were treated with hypoxia and transforming growth factor β (TGFβ), two factors that both contribute to the immunosuppression of TME, for the assessment of their sustained tumor-killing activity. Emami Nejad et al., The role of hypoxia in the tumor microenvironment and development of cancer stem cell: a novel approach to developing treatment, Cancer Cell Int'l 21: 63 (2021); Lequeux et al., Impact of hypoxic tumor microenvironment and tumor cell plasticity on the expression of immune checkpoints, Cancer Letters 458: 13-20 (2019).

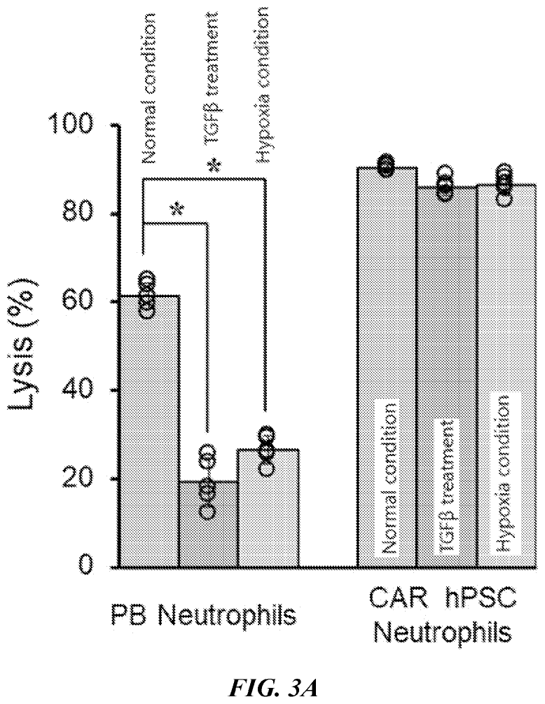

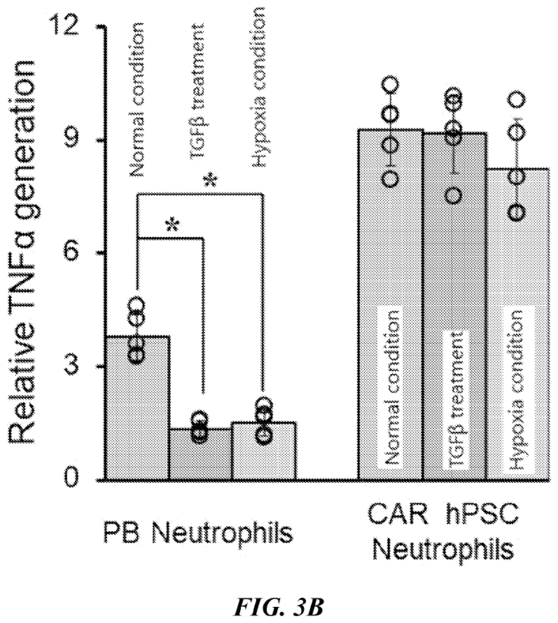

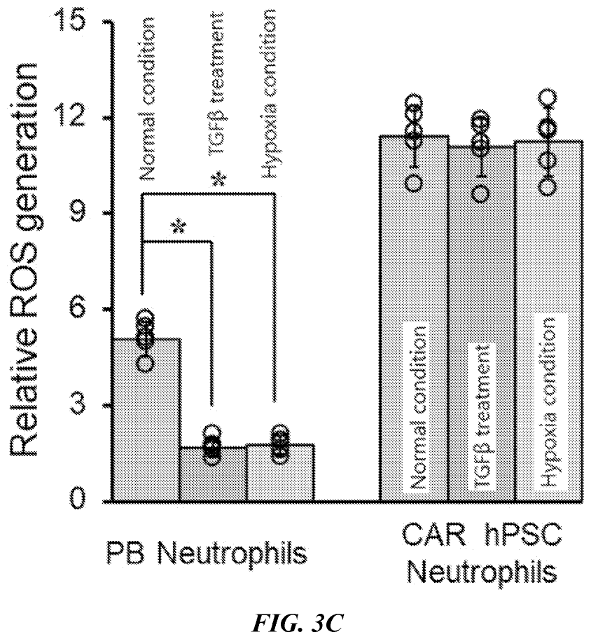

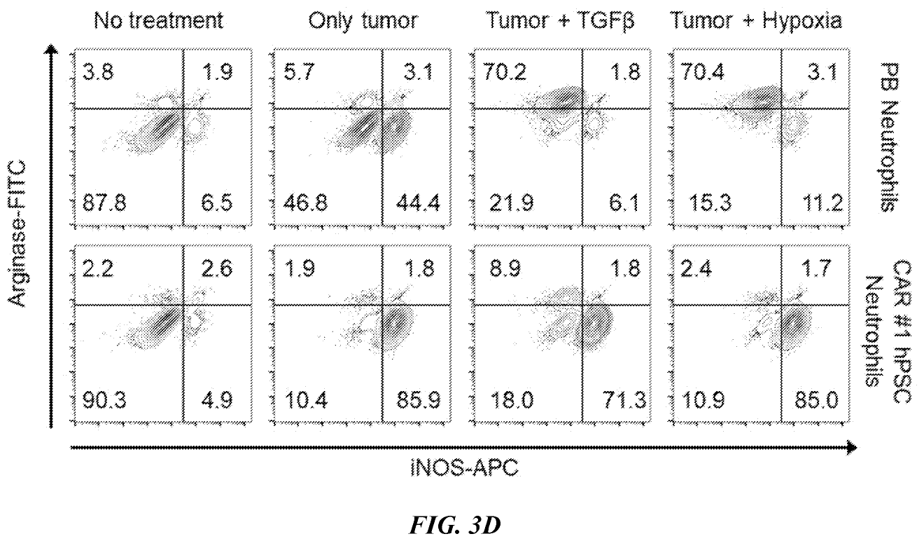

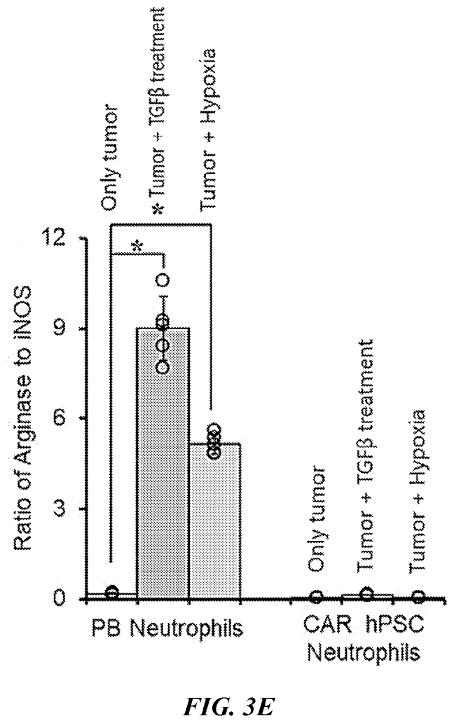

While PB neutrophils presented significantly decreased cytolysis against GBM cells under immunosuppressive than normal conditions, CAR-neutrophils sustained high tumor-killing activities under both hypoxic (3% 02) and TGFβ conditions (FIG. 3A). Similar observations were also made in the TNFα release and ROS generation (FIGS. 3B and 3C) from PB or CAR-neutrophils under immunosuppressive and normal conditions.

To further confirm neutrophil phenotype under hypoxic and TGFβ conditions, the expression of N1-specific iNOS and N2-specific arginase on isolated neutrophils was examined by flow cytometry (FIGS. 3D and 3E). Compared with normoxia, immunosuppressive hypoxia and TGFβ significantly decreased expression levels of iNOS and increased levels of arginase in PB neutrophils, whereas CAR-neutrophils retained high expression levels of iNOS under hypoxic and TGFβ conditions. Taken together, CAR-neutrophils sustained an anti-tumor phenotype and retained high anti-tumor activities under TME-mimicking conditions in vitro, highlighting their potential in targeted immunotherapy.

### Example 3

Preparation and Characterization of hPSC CAR-Neutrophils Loaded with Tirapazamine (TPZ)-Containing SiO₂ Nanoparticles

PB neutrophils have been used as cellular carriers to deliver imaging and therapeutic drugs into brain tumors, though targeted neutrophil infiltration requires surgery- or light-induced inflammation, and off-target drug delivery may be a concern. Xue et al. (2017), supra; Wu et al. (2018), supra; Chu et al. (2017), supra; Osuka & Van Meir (2017), supra. To further improve anti-tumor activities of the CAR-neutrophils, silica nanoparticles (SiO₂—NP) with rough or smooth surfaces were prepared (pursuant to the protocols set forth above in the Materials and Methods section) to load chemotherapeutic or radiation drugs into neutrophils.

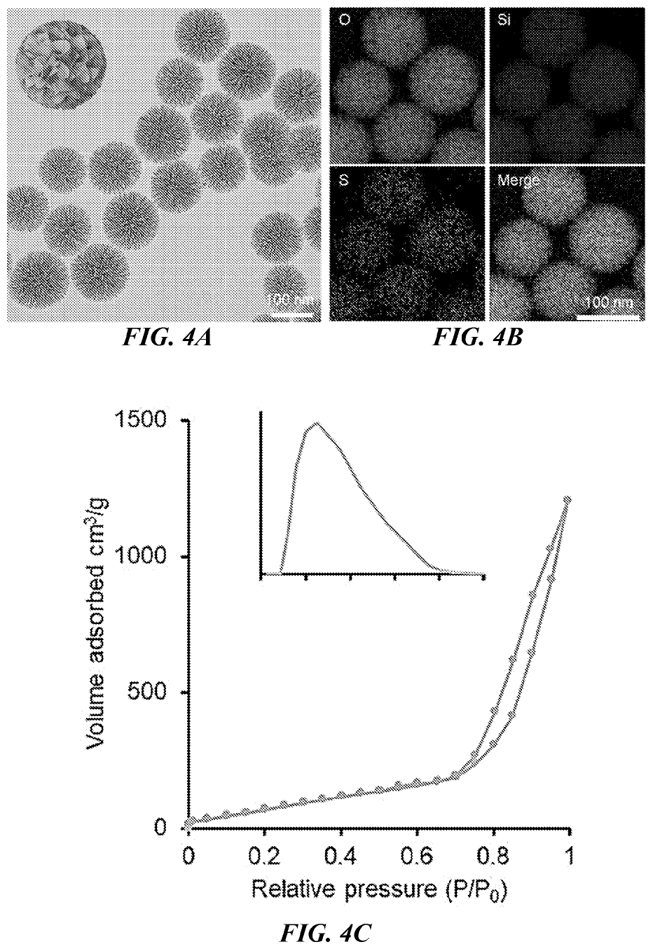

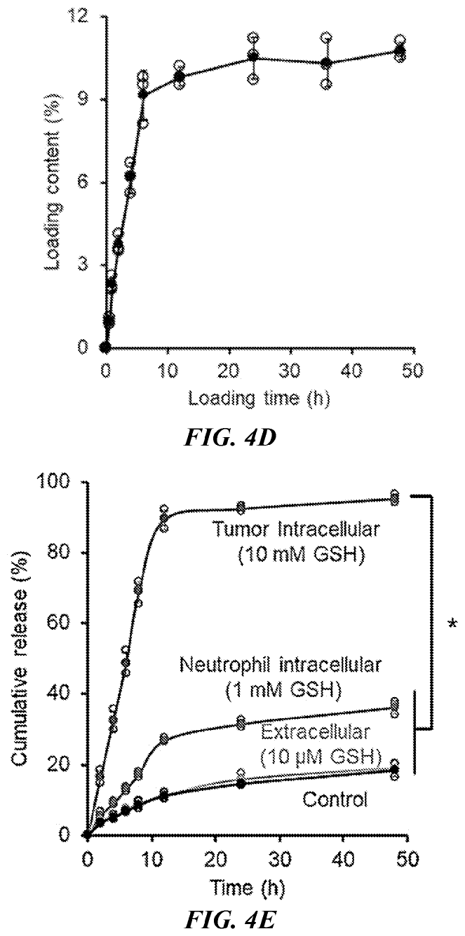

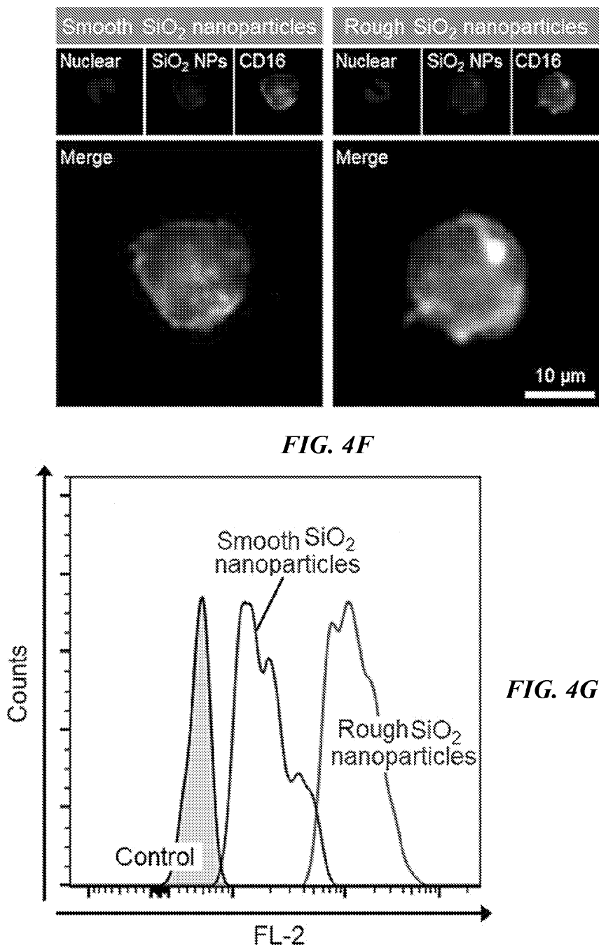

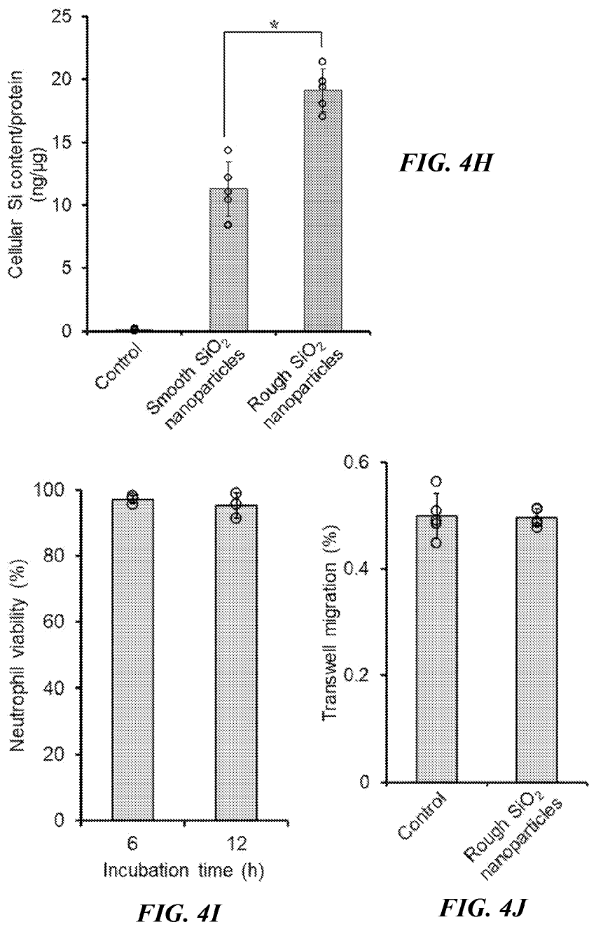

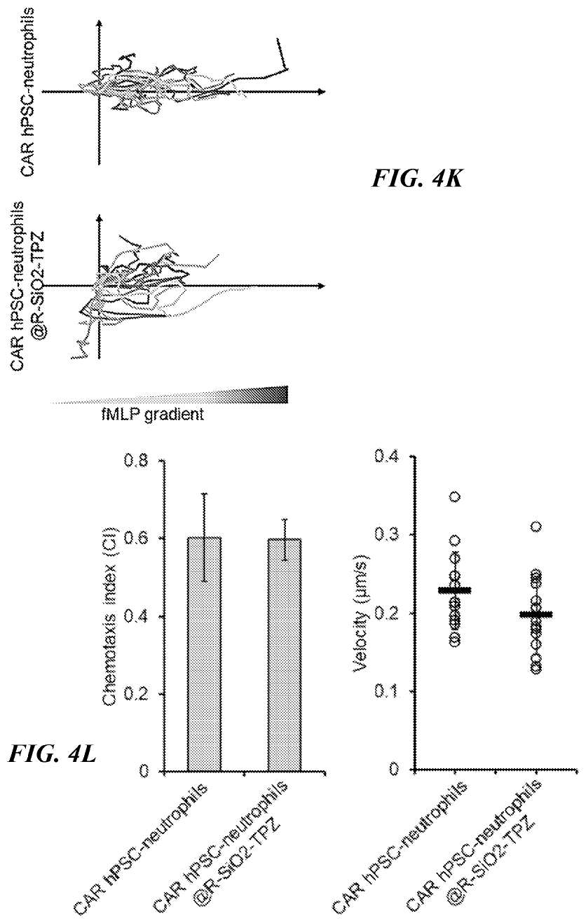

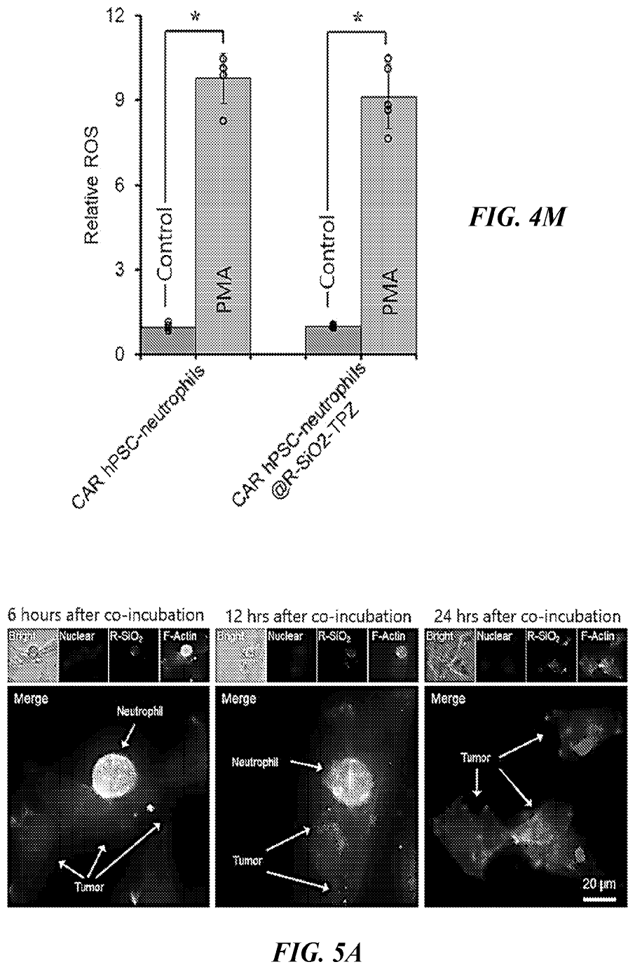

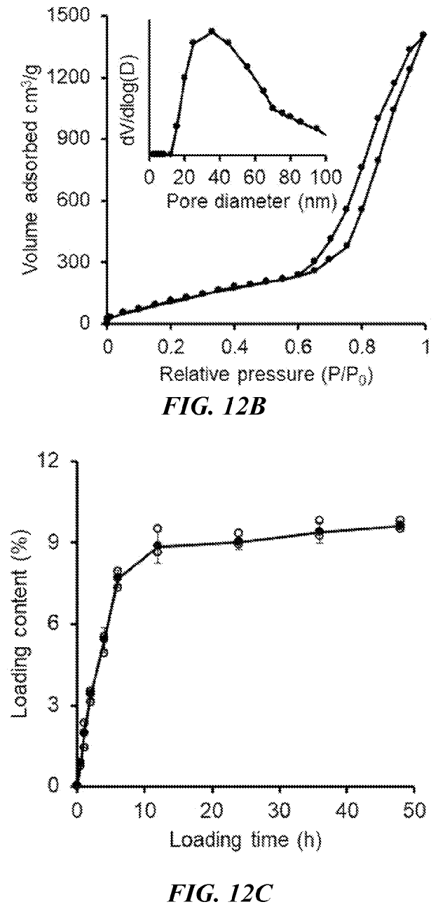

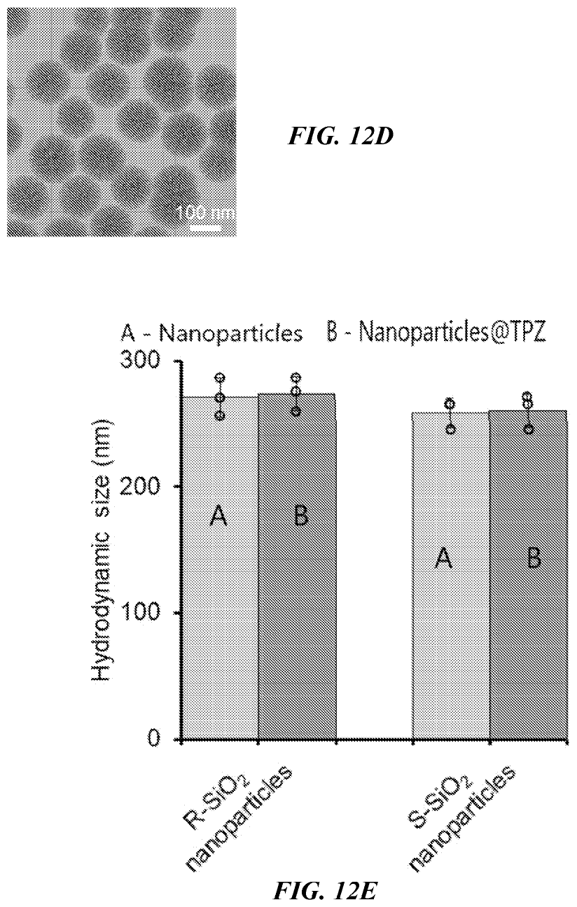

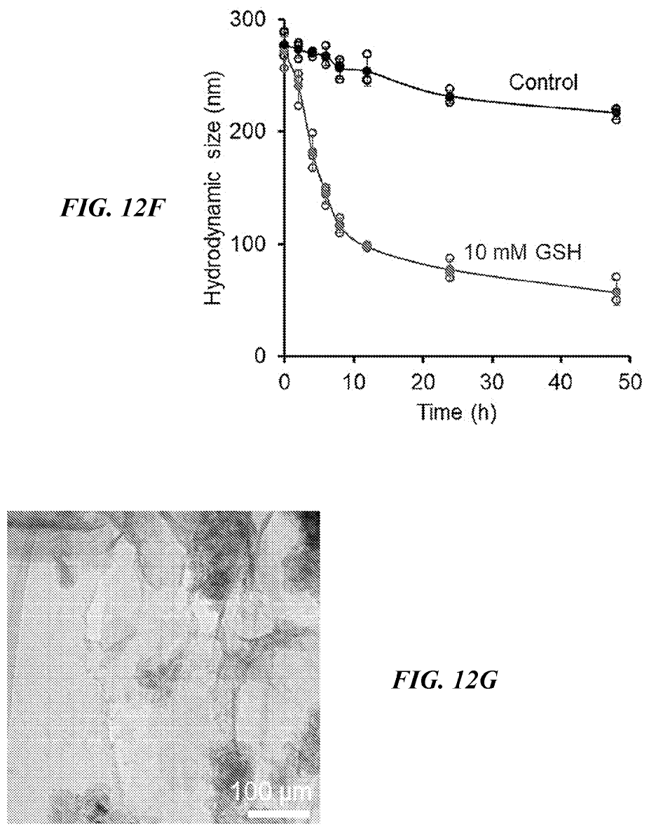

Transmission electron microscope (TEM) images demonstrated that both rough and smooth SiO₂ nanoparticles were well-dispersed and exhibited spherical morphology with uniform size (FIGS. 4A and 12A). Composition distribution analysis via scanning TEM (STEM) with energy-dispersed X-ray spectroscopy (EDS) showed that sulfur (S) was evenly distributed within the whole rough SiO₂ nanoparticles (R—SiO₂) (FIG. 4B). Using nitrogen (N2) adsorption-desorption isotherms and corresponding pore size distribution analysis, pore sizes of rough (R-) and smooth (S-) SiO₂ NPs were measured as 25 nm and 35 nm (FIGS. 4C and 12B), respectively. Given the high surface area and large pore size, therapeutic drugs could be effectively loaded into both R- and S—SiO₂ NPs, as exemplified by the hypoxia-responsive prodrug tirapazamine (TPZ) (FIGS. 4D and 12C).

Briefly, for tirapazamine (TPZ) loading, 5 mL of 1 mg mL⁻¹ TPZ in phosphate buffer solution were mixed with 5 mL of SiO₂ suspension (5 mg mL⁻¹) in phosphate buffer solution (20 mM, pH=7.4), and the resulting solution was continuously stirred at 37° C. for different time points (0.5, 1, 2, 4, 6, 12, and 24 hours). Unloaded TPZ was removed by centrifugation at 8,000 rpm for 10 minutes, and the pellet was washed with phosphate buffer solution three times. TPZ in the supernatant was determined by UV-Vis spectroscopy. TPZ loading capacity (LC) was calculated as follows: LC=(total TPZ-TPZ in supernatant)/(total TPZ)×100%. For glutathione (GSH)-stimulated release analysis, 10 mL of TPZ@SiO₂ suspension in phosphate buffer solution (1 mg mL⁻¹) were incubated with 10 mM GSH at different time points (10, 20, 30, 40, 50, and 60 hours). One mL of the complex dispersion was removed and centrifuged at 8,000 rpm for 10 min, and TPZ released to the supernatant was quantified by UV-Vis spectroscopy. Similar procedures were performed to load TMZ and JNJ64619187.

After TPZ loading, significant changes were not observed in the dispersity, morphology, and size of R—SiO₂-TPZ using TEM and dynamic light scattering analysis (FIGS. 12D and 12E). The tetra-sulfide bonds incorporated into the R—SiO₂ NPs are sensitive to reductive environments and can rapidly degrade by the large amount of glutathione (GSH) presented within the tumor cells. Liu et al, A Tumor-Microenvironment-Responsive Nanocomposite for Hydrogen Sulfide Gas and Trimodal-Enhanced Enzyme Dynamic Therapy, Advanced Materials 33(30): 2101223 (2021).

GSH-responsive degradability of R—SiO₂-TPZ NPs in the presence of 10 mM, 1 mM, and 10 μM GSH, which were the same as the intracellular conditions of cancer cell, normal cell, and extracellular environments, respectively, was determined next. Liu et al (2021). supra. Upon 10 mM GSH treatment, the initial spherical structure of R—SiO₂-TPZ NPs was severely destructed after 24 hours (FIGS. 12F and 12G), and the nanoparticles were completely disintegrated into small debris after 48 hours, resulting in TPZ release in a GSH-responsive manner (FIG. 4E).

The feasibility of using SiO₂-TPZ NPs to load therapeutic drugs into CAR-neutrophils as a combinatory chemoimmunotherapy to achieve boosted therapeutic efficacy was examined. Briefly, nanodrug loading was prepared by incubating neutrophils with TPZ@SiO₂ or nanoparticles loaded with TMZ or JNJ64619187. (TMZ and JNJ64619187 were each loaded into SiO₂ NPs using similar procedures to that described above in connection with TPZ).

Briefly, hPSC-derived neutrophils were placed in a DNA low-bind tube and incubated with nanodrugs for 1 hour. After centrifugation and PBS washing three times, nanodrug/neutrophils were resuspended in PBS and readied for subsequent experiments. Cellular uptake efficiency of SiO₂-TPZ NPs by neutrophils was measured by flow cytometry analysis, demonstrating a larger cellular uptake of R—SiO₂-TPZ NPs than S—SiO₂-TPZ NPs by neutrophils. The location of nanodrugs within the neutrophils was determined by fluorescence microscope. Neutrophil viability after incubating with nanodrugs for 4 and 8 hours was measured by Zombie Green Fixable Viability Kit (BioLegend, San Diego, CA). (FIGS. 4F-4G).

To quantify the loading content of SiO₂, nanodrug/neutrophil samples were digested by tetramethylammonium hydroxide and a high pressure, and the silicon concentrations of digested samples were measured by inductively coupled axial plasma optical emission spectrometry (ICP-OES). Cellular Si content in neutrophils was measured as 11.3 and 19.1 ng Si/g protein for smooth and rough SiO₂ NPs@TPZ (FIG. 411), respectively. Given their high loading capacity in neutrophils, R—SiO₂-TPZ NPs were employed for subsequent experiments.

The physiological functions of CAR-neutrophils after loading R—SiO₂-TPZ NPs was then tested. Primarily, cell viability was analyzed by flow cytometry. Additionally, a Transwell migration assay was performed. Neutrophils were resuspended in HBSS buffer and allowed to migrate for 2 hours towards fMLP (10 nM and 100 nM). Cells that migrated to the lower chamber were released with 0.5 M EDTA and quantified by Accuri C6 plus cytometer (Beckton Dickinson & Company, Franklin Lakes, NJ). Live neutrophils were gated and analyzed in the FlowJo software. Neutrophil counts were then normalized by the total number of cells added to each well.

A 2D chemotaxis assay was also performed. Neutrophils were resuspended in HBBS with 20 mM HEPES and 0.5% fetal bovine serum (FBS) and loaded into collagen-coated IBIDI chemotaxis μ-slides, which were then incubated at 37° C. for 30 minutes for cells to attach. 15 L of 1,000 nM N-formylmethionyl-leucyl-phenylalanine (fMLP) were loaded into the right reservoir to yield a final fMLP concentration of 187 nM. Cell migration was recorded every 60 seconds for a total of 120 minutes using LSM 710 (with Ziess EC Plan-NEOFLUAR 10×/0.3 objective) at 37° C. Cells were tracked with ImageJ plug-in MTrackJ (publicly available).

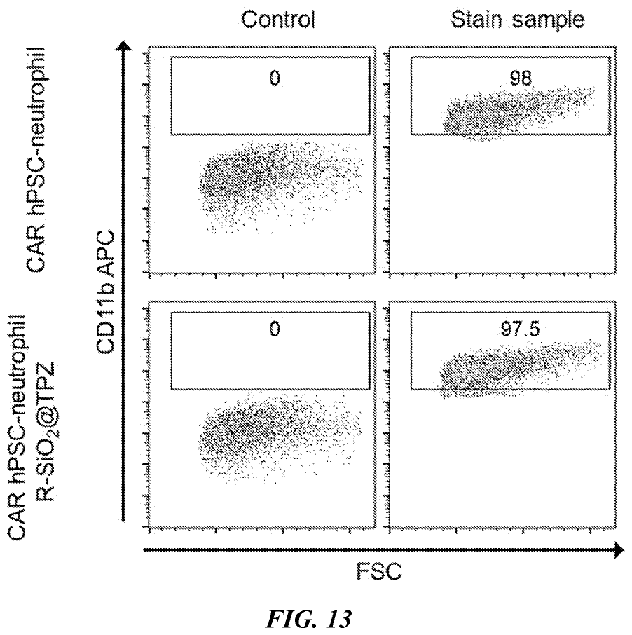

Significant changes were not observed in cell viability (FIG. 4I), Transwell migration ability (FIG. 4J), chemotaxis and corresponding velocity (FIGS. 4K-4L) of CAR-neutrophils before or after loading R—SiO₂-TPZ NPs, demonstrating their high biocompatibility. The expression level of CDT1b, a neutrophil surface protein that mediates adhesion and migration function upon inflammatory molecule stimulation, was not changed on CAR-neutrophils with or without R—SiO₂-TPZ loading (FIG. 13) (determined via flow cytometry).

Superoxide or reactive oxygen species (ROS) are released from active neutrophils to kill microbes and tumor cells. Nguyen et al., Neutrophils to the ROScue: Mechanisms of NADPH oxidase activation and bacterial resistance, Frontiers in Cellular & Infection Microbiology 7 (2017). Accordingly, ROS production was assessed using protocols known in the art. As expected, ROS generation by CAR-neutrophils was significantly increased after N-formylmethionine-leucyl-phenylalanine (fMLP) treatment, and significant differences were not observed in ROS production by CAR-neutrophils before and after loading R—SiO₂-TPZ (FIG. 4M).

In sum, the data supports that R—SiO₂-TPZ loaded CAR neutrophils maintained the physiological activities of wild-type neutrophils and can actively migrate towards inflammatory stimuli, highlighting their potential in targeted cancer chemoimmunotherapies.

### Example 4

CAR-Neutrophils Loaded with R—SiO₂-TPZ Nanoparticles Effectively Kill GBM Cells

The effect of R—SiO₂-TPZ on tumor-killing ability of CAR-neutrophils was evaluated. Intimate effector-target interaction was a prerequisite for neutrophil-mediated cytolysis.

To visualize immunological synapses, 100 μL of U87MG cells (50,000 cells/mL) were seeded onto wells of 96-well plate and incubated at 37° C. for 12 hours to allow cells to attach. Neutrophils (100 μL; 500,000 cells/mL) were then added onto the target U87MG cells and incubated for 6 hours before fixation with 4% paraformaldehyde (in PBS). Cytoskeleton staining was then performed using an F-actin Visualization Biochem Kit (Cytoskeleton Inc., Denver, CO). Somatic cells were also used as a control, following similar protocols as outlined above.

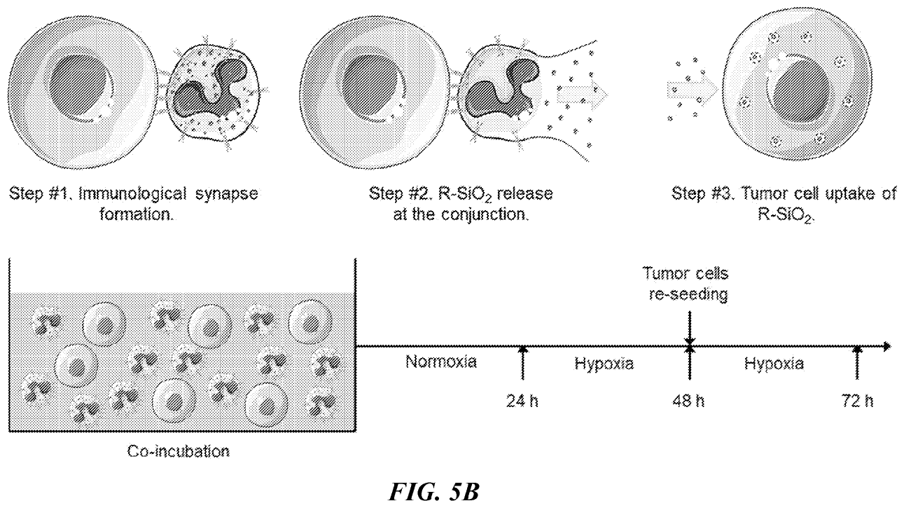

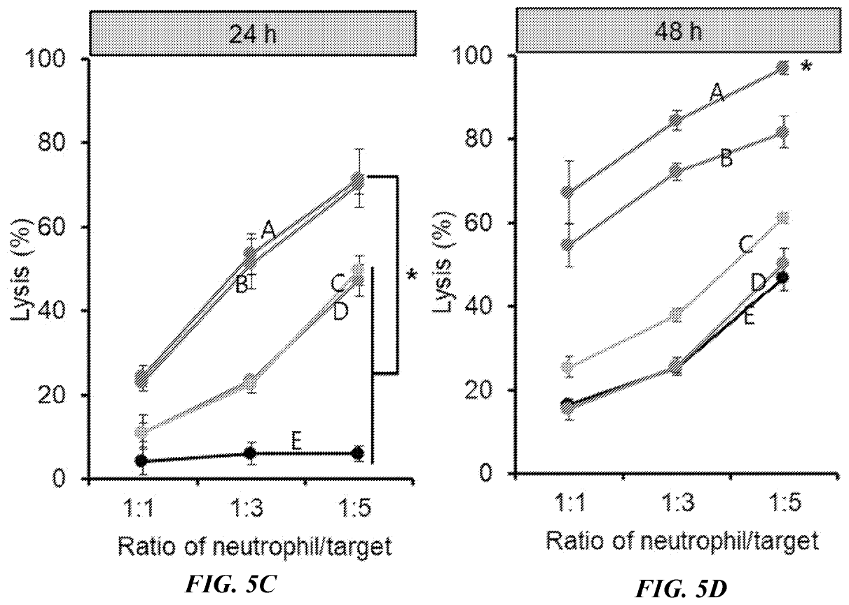

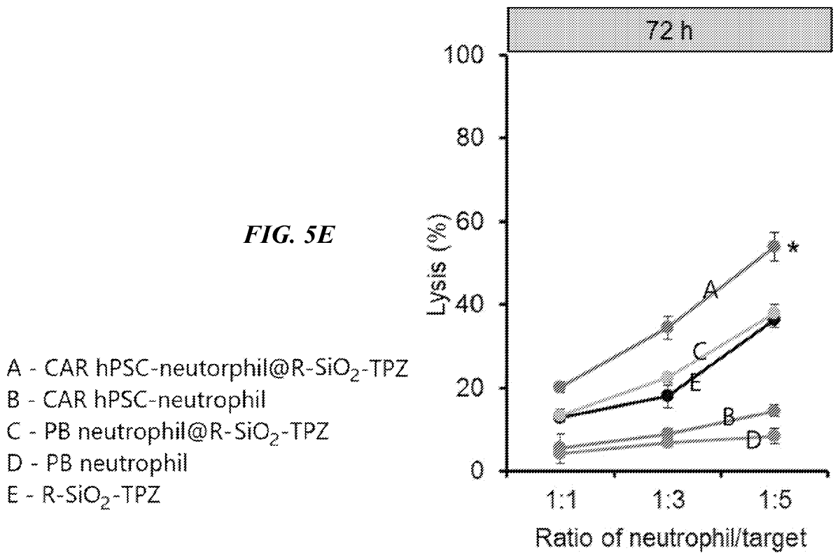

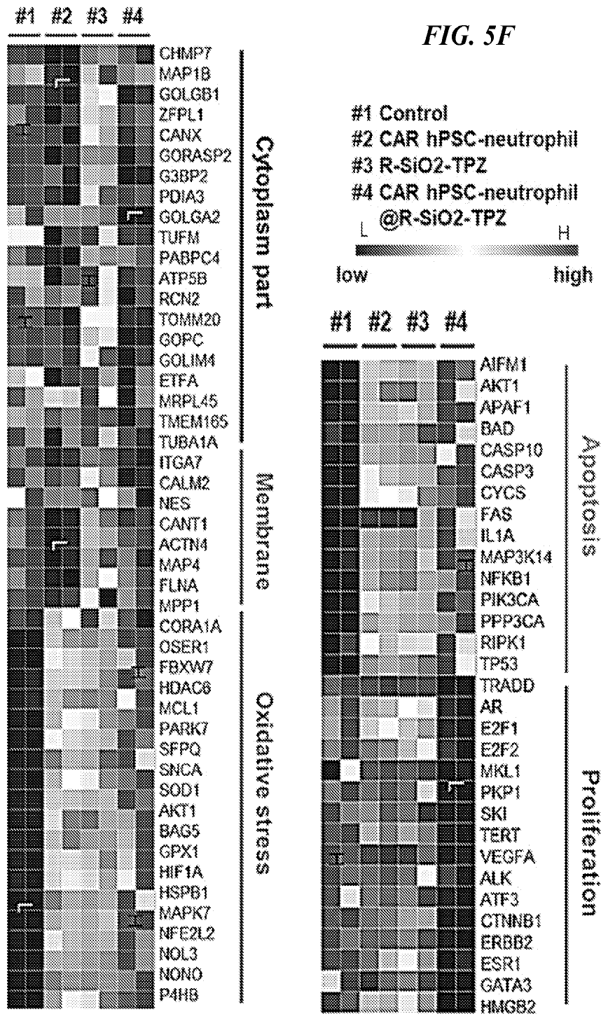

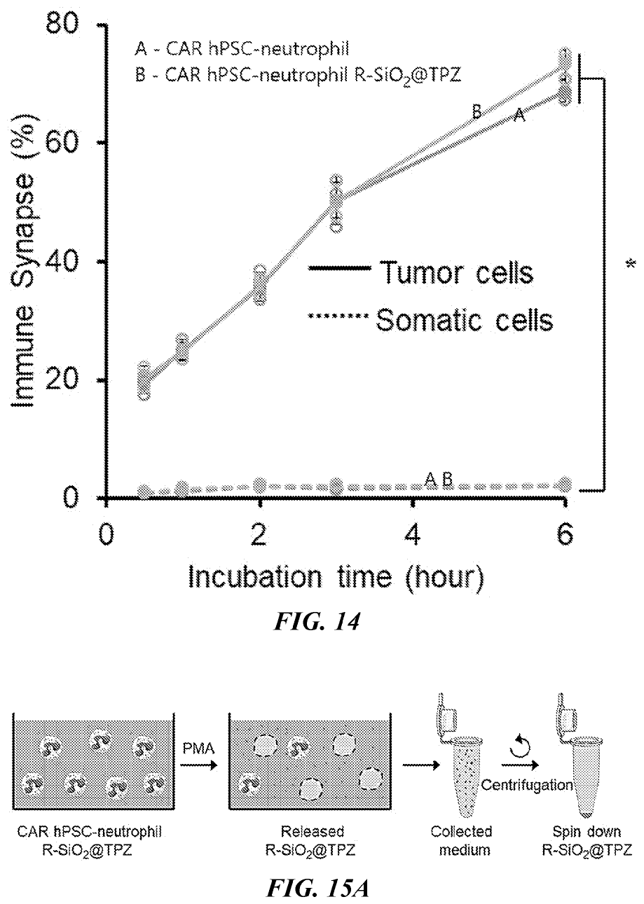

As expected, CAR-neutrophils@R—SiO₂-TPZ formed immune-synapse with tumor cells within 2 hours and exhibited similar effector-target interaction numbers as drug-free CAR-neutrophils (FIG. 5A and FIG. 14). Notably, no observable interactions were formed between CAR neutrophils @JR-SiO₂-TPZ and normal somatic cells (FIG. 14), highlighting the specificity of CLTX-CAR against brain tumors.

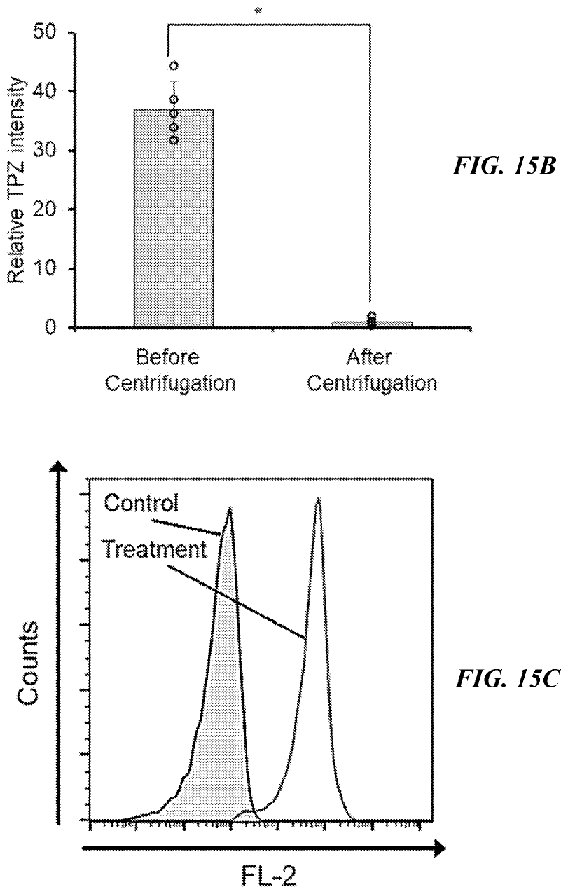

Furthermore, R—SiO₂-TPZ NPs were released from neutrophils into culture medium with intact morphology (FIGS. 15A and 15B) 12 hours after co-culture and were partially taken up by remaining tumor cells (FIG. 5A). 24 hours after co-incubation of SiO₂-TPZ NP-loaded CAR neutrophils with tumor cells, up to 95% of tumor cells took up R—SiO₂-TPZ NPs (FIGS. 5A and 15C), indicating a successful transport cascade involving carrier neutrophils that undergo rupture, exert effector cell function, and release and deliver R—SiO₂-TPZ NPs to the target tumor cells. To determine the cytolysis of R—SiO₂-TPZ NP-loaded CAR-neutrophils, an in vitro normoxia-hypoxia tumor rechallenging model was implemented (FIG. 5B). 24 hours after normoxic co-culture, CAR-neutrophils loaded with or without R—SiO₂-TPZ NPs exhibited similar anti-tumor cytotoxicity (FIG. 5C), and both were higher than those of PB-neutrophil loaded with or without R—SiO₂-TPZ NPs and R—SiO₂-TPZ NPs alone. This is mainly due to the enhanced tumor-targeting ability of neutrophils after CAR engineering.

After an additional 24-hour hypoxic co-culture with tumor cells, R—SiO₂-TPZ NP-loaded CAR-neutrophils displayed superior anti-tumor ability compared to other groups (FIG. 5D). CAR-neutrophils loaded with R—SiO₂-TPZ NPs also exhibited excellent cytolysis against re-seeded tumor cells (FIG. 5E).

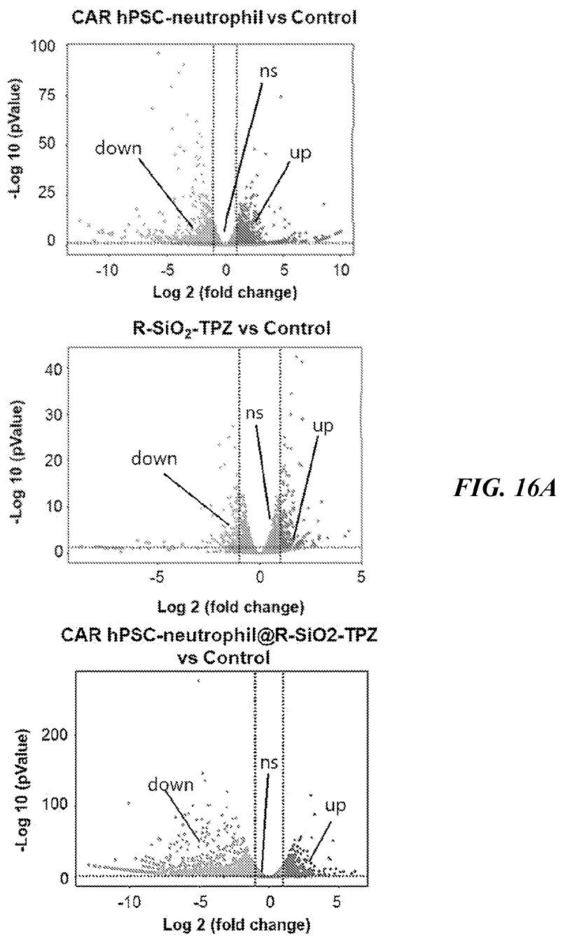

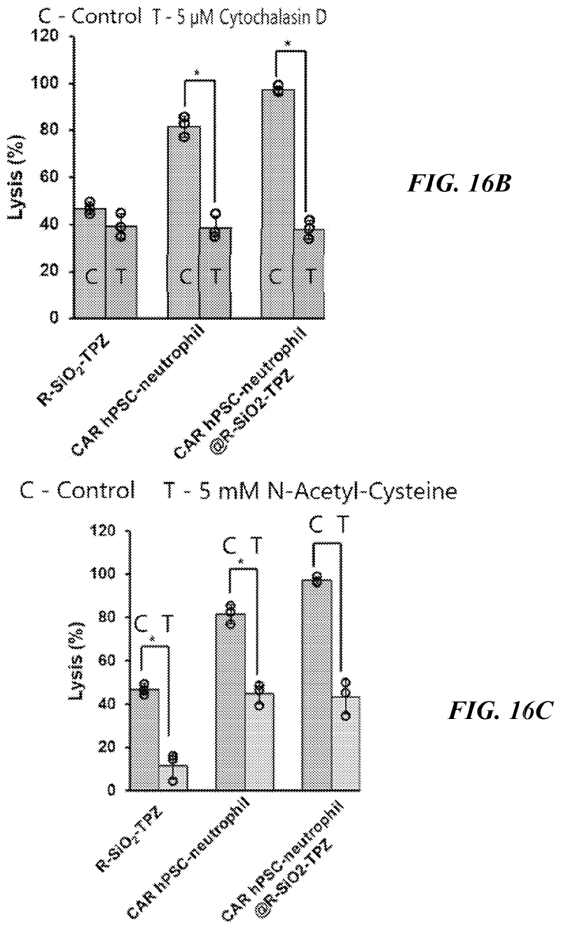

RNA sequencing (RNA-seq) analysis on tumor cells was performed to elucidate potential molecular mechanism underlying enhanced anti-tumor cytolysis of neutrophils by CAR expression and R—SiO₂-TPZ NP. Gene expression analysis demonstrated that as compared to control and R—SiO₂-TPZ NP, CAR-neutrophils loaded with or without R—SiO₂-TPZ NPs significantly decreased the cytoplasm and membrane part of tumor cells (FIG. 16A and FIG. 5F), further supporting their phagocytosis of tumor cells upon co-culture. While all experimental groups increased cellular oxidative stress in tumor cells, R—SiO₂-TPZ loaded CAR-neutrophils outperformed other groups in triggering oxidative stress signaling. In addition, R—SiO₂-TPZ loaded CAR-neutrophils significantly promoted apoptosis and decreased proliferation in tumor cells. To further understand enhanced anti-tumor activities of R—SiO₂-TPZ-loaded CAR-neutrophils, a phagocytosis inhibitor cytochalasin D and a reactive oxygen species (ROS) inhibitor N-acetyl-cysteine (NAC) were applied to the tumor-neutrophil co-culture. Cytolysis of tumor cells by CAR-neutrophils was significantly reduced by 5 μM cytochalasin D (FIG. 16B) and 5 mM NAC (FIG. 16C), indicating the prominent role of phagocytosis and ROS in CAR neutrophil-mediated tumor-cell killing.

### Example 5

Functional Evaluation of CAR-Neutrophils Loaded with Nanodrugs Using Biomimetic GBM Models In Vitro

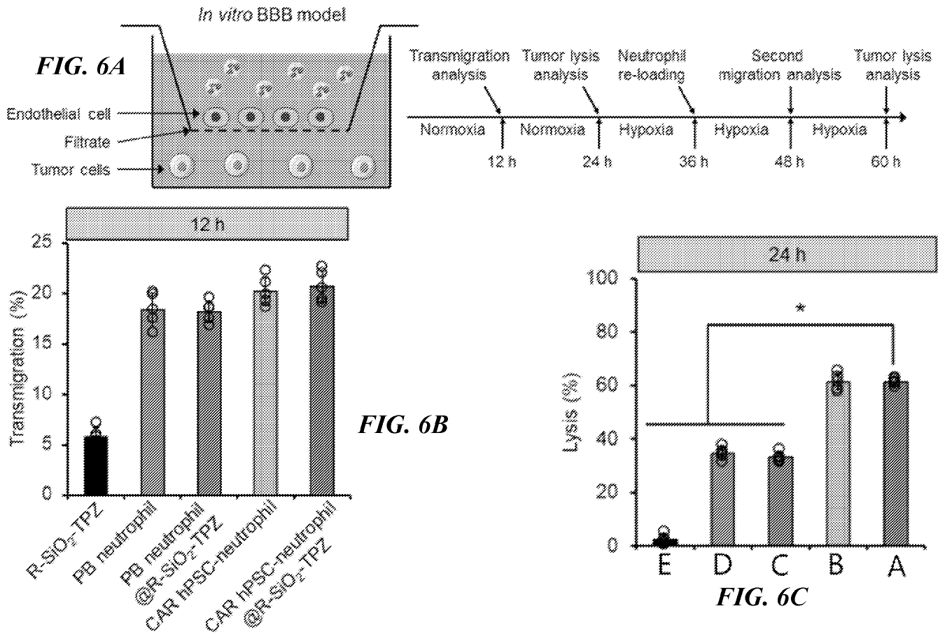

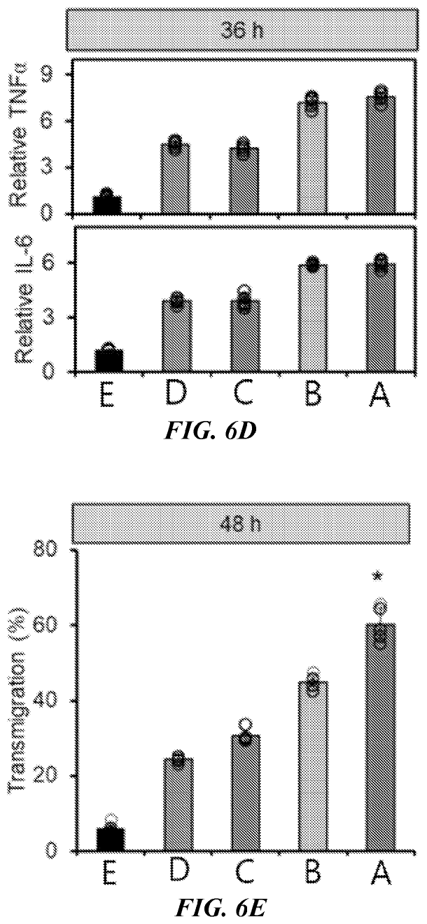

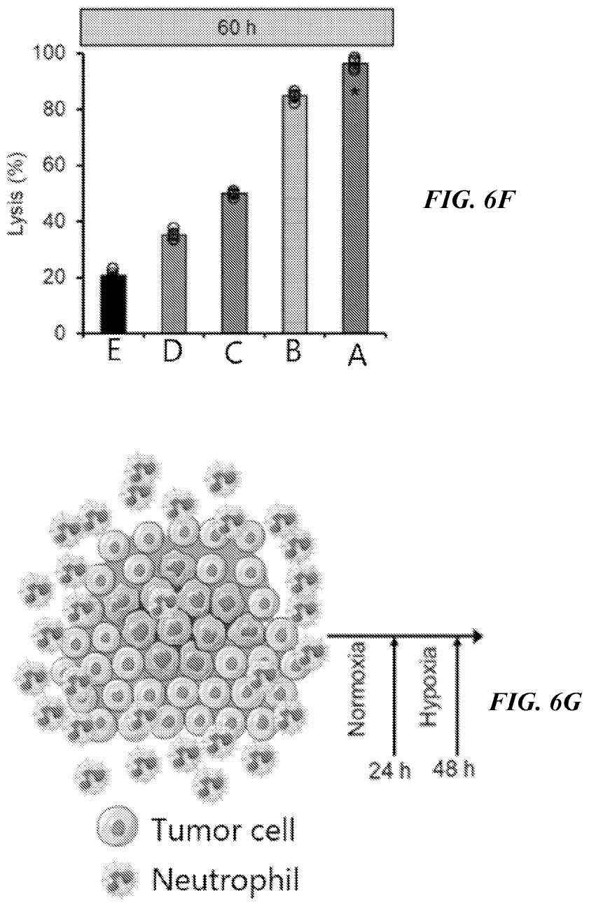

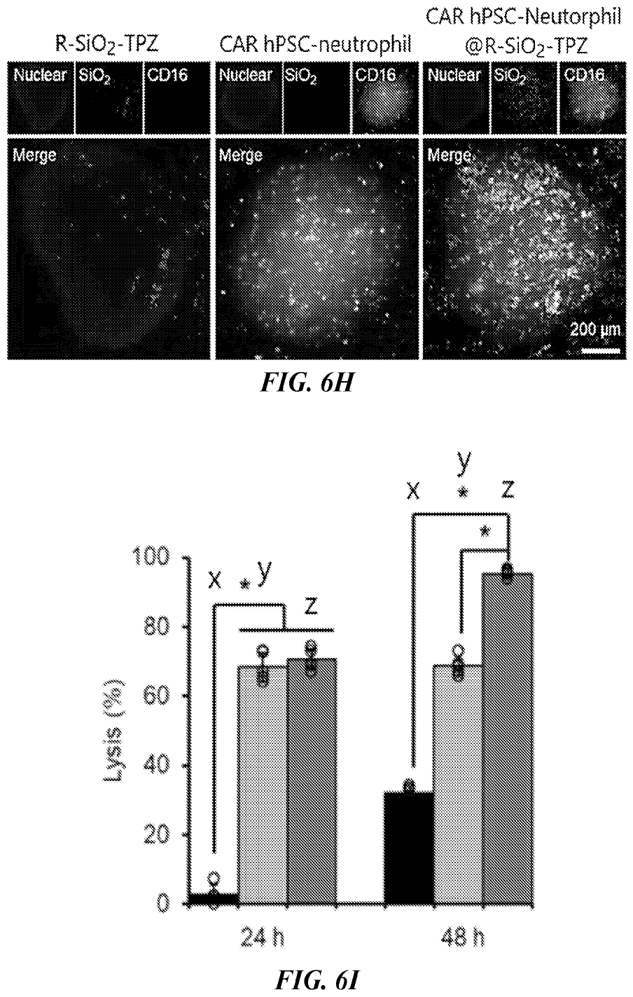

To further assess the activities of R—SiO₂-TPZ NP-loaded CAR-neutrophils, a transwell-based blood brain barrier (BBB) tumor model using human cerebral microvascular endothelial cells was implemented (FIG. 6A). An in vitro BBB model was constructed with HBEC-5i cells in a transwell cell culture plate. Briefly, HBEC-5i cells (1×10⁵ cells/well) were seeded onto the upper chamber of the transwell pre-coated with gelatin (1% w:v) in 24-well transwell plates (8 m pore size, 6.5 mm diameter, Corning), and maintained in DMEM/F12 medium containing 10% FBS. Neutrophils (2×10⁵) were then added to the upper chamber, and FBS-free medium with or without 10 nM fMLP was added to the lower chamber. After 3 hours of incubation, cell cultures were collected from the upper or lower chamber to calculate neutrophil numbers. For the cytotoxicity analysis, 2×10⁴ U87MG cells were seeded at the lower chamber 12 hours before adding neutrophils (2×10⁵ cells) to the upper chamber, and FBS-free medium with 10 nM fMLP was then added to the lower chamber. After 12 hours of incubation, tumor cell viability was determined by flow cytometry analysis. For the second migration analysis, 2×10⁵ neutrophils from the bottom chamber of first migration were seeded in the upper chamber of a second transwell BBB model, and the neutrophils that migrated towards target tumor cells in the bottom chamber were quantified.

As expected, R—SiO₂-TPZ NP-loaded CAR-neutrophils exhibited excellent transmigration ability across in vitro BBB model (FIG. 6B), effectively killed targeted tumor cells after transmigration (FIG. 6C) and released more inflammatory cytokines (FIG. 6D) that may attract other effector cells to kill tumor. R—SiO₂-TPZ NP-loaded CAR-neutrophils retained excellent transmigration ability during a second transmigration experiment (FIG. 6E) and superior anti-tumor ability compared with other groups (FIG. 6F).

A three-dimensional (3D) tumor spheroid model was then employed to evaluate the tumor-penetration ability of R—SiO₂-TPZ NP-loaded CAR-neutrophils (FIG. 6G). 3D tumor spheroids were generated by the hanging-drop method. Briefly, U87MG cells were suspended in minimum essential medium (MEM) medium with 10% FBS and 0.3% methylcellulose at 2×10⁶ cells/mL and deposited onto an inverted lid of 96-well plate as an individual drop using a 20 μL pipettor. The cover lid was then placed back onto the PBS-filled bottom chamber, and the whole plate was incubated at 37° C. and 5% CO₂. The hanging drops were monitored daily until cell aggregates were formed in 5-7 days. Each cell aggregate was transferred to a single well of a 24-well plate for subsequent experiments.

To assess the tumor penetration capability of CLTX-CAR neutrophils, 2×10⁵ neutrophils/well were added to the wells of 24-well plate and incubated with tumor spheroids. After co-incubation for 24 hours, tumor spheroids were fixed and stained for CD45 and DAPI. For cytotoxicity analysis, both live/dead cell staining and the CytoTox-Glo™ Cytotoxicity Assay kit (Promega Corporation, Madison, WI) were employed. The CytoTox-Glo™ Cytotoxicity Assay was quantified by the SpectraMax iD3 microplate reader (Molecular Devices, Sunnyvale, CA).

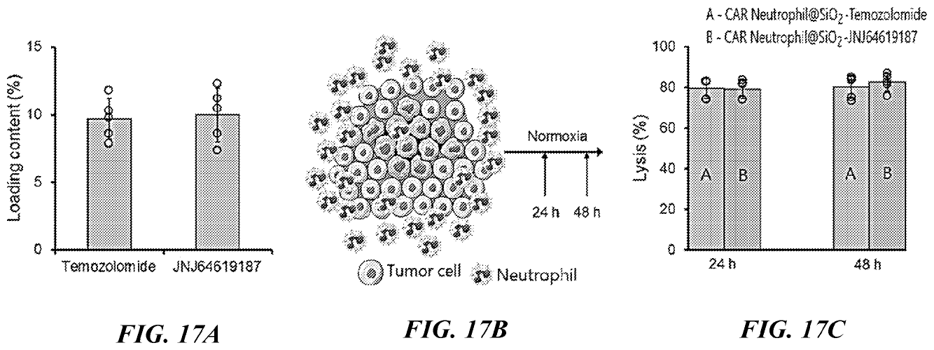

CAR-neutrophils gradually migrated toward the center of a tumor organoid and uniformly distributed in the tumor organoid after 8 hours of incubation (FIG. 6H). A high degree of co-localization between CAR-neutrophils and R—SiO₂-TPZ NPs was observed, demonstrating that R—SiO₂-TPZ NPs were encapsulated stably in the CAR-neutrophils during tumor infiltration. Without neutrophil-mediated delivery, R—SiO₂-TPZ NPs were only found on the outside layer of tumor spheroids. As compared to R—SiO₂-TPZ NPs and CAR-neutrophils, R—SiO₂-TPZ NP-loaded CAR-neutrophils exhibited superior anti-tumor cytolysis in the 3D tumor model (FIG. 6I). CAR-neutrophils@R—SiO₂ NPs can also be employed to deliver other drugs, including clinical temozolomide (TMZ) and JNJ64619187, into 3D tumor models and efficiently kill GBM cells (FIGS. 17A-17C). Taken together, the combinatory CAR neutrophils and nanodrugs displayed excellent anti-tumor activities in biomimetic TME-mimicking conditions in vitro, highlighting the therapeutic potential of combinatory neutrophil-based chemoimmunotherapy.

### Example 6

### In Vivo Distribution of CAR Neutrophil-Delivered R—SiO₂-TPZ Nanoparticles

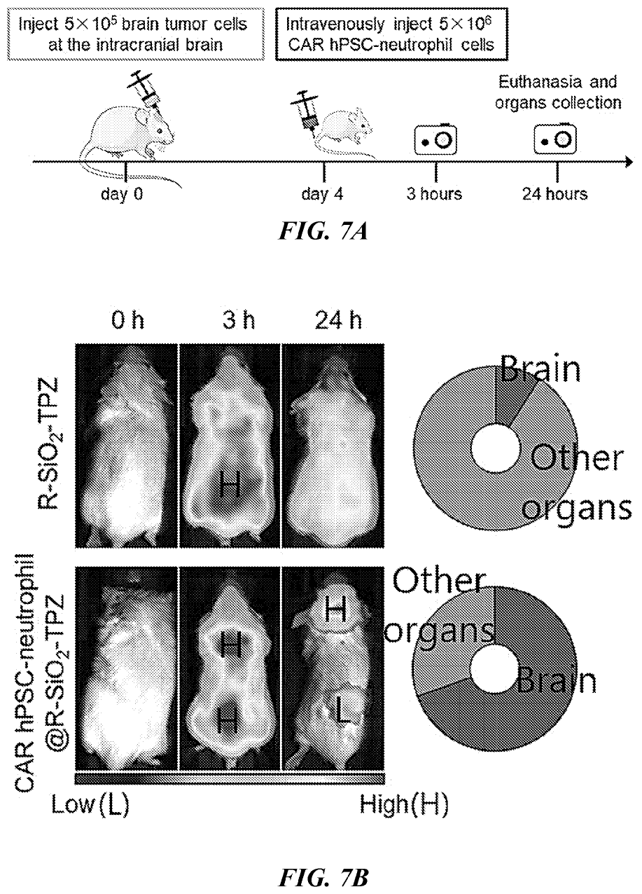

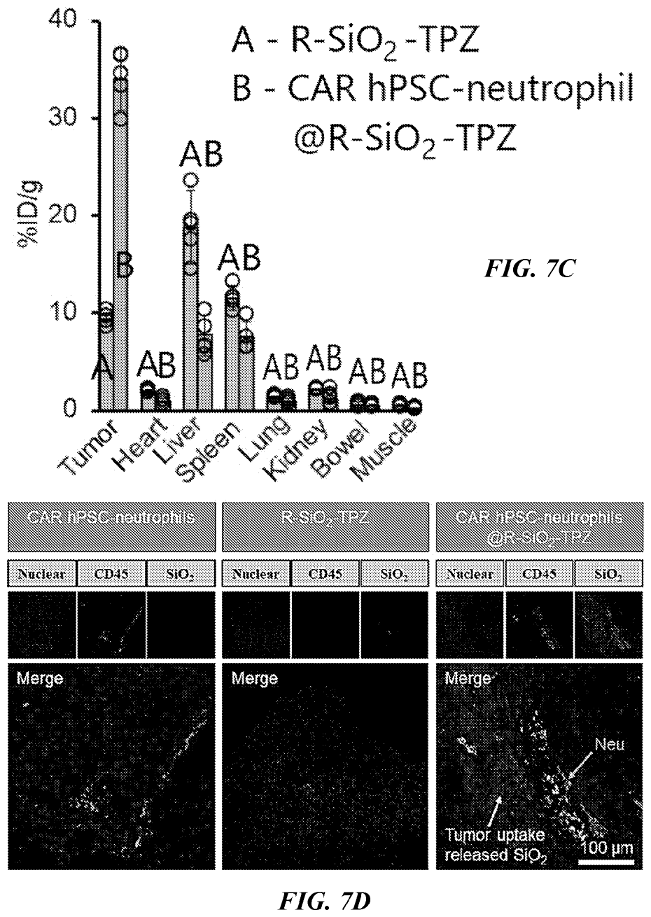

In addition to the improvement of direct tumor-killing ability, it was hypothesized that CAR engineering of hPSC-neutrophils will significantly enhance their targeted delivery of therapeutic drugs without additional surgery- or light-induced inflammation. Osuka et al. (2017), supra. To test this hypothesis, a mouse xenograft model of glioblastoma and an in vivo imaging system were employed to determine the trafficking and biodistribution of R—SiO₂-TPZ NP-loaded CAR-neutrophils. SiO₂ NPs were fluorescently labeled with the near-infrared dye Cyanine 5 (Cy5), and fluorescence imaging was performed at 3 and 24 hours after systemic administration (FIG. 7A). Only 3 hours after intravenous injection, R—SiO₂-TPZ NPs travelled to the whole body of tumor-bearing mice and emitted strong fluorescence with or without neutrophil-mediated delivery (FIG. 7B).

As expected, CAR-neutrophil-delivered R—SiO₂-TPZ NPs eventually accumulated in the brain tumor site within 24 hours, whereas non-neutrophil-delivered R—SiO₂-TPZ NPs were still evenly distributed across the whole body (FIG. 7B). To further quantify biodistribution of R—SiO₂-TPZ NPs in various organs, inductively coupled plasma-optical emission spectrometry (ICP-OES) analysis of Si content was performed on the harvested organs 24 hours post-injection and confirmed that CAR neutrophil-delivered R—SiO₂-TPZ NPs were significantly enriched in the mouse brain (FIG. 7C), though minimal off-target delivery to liver and spleen was observed. Targeted delivery of R—SiO₂-TPZ NPs to the host brain across BBB by CAR-neutrophils was also confirmed by histology analysis (FIG. 7D). On the contrary, R—SiO₂-TPZ NPs alone mainly accumulated in the liver and spleen. Collectively, the data demonstrated enhanced targeted delivery of R—SiO₂-TPZ NPs by CAR-neutrophils without inducing additional inflammation, highlighting the feasibility and safety of neutrophil-based chemoimmunotherapy in cancer treatment.

### Example 7

### The Combinatory Chemoimmunotherapy of CAR-Neutrophils and R—SiO₂-TPZ Nanoparticles Exhibited Excellent Anti-Glioblastoma Activities In Vivo

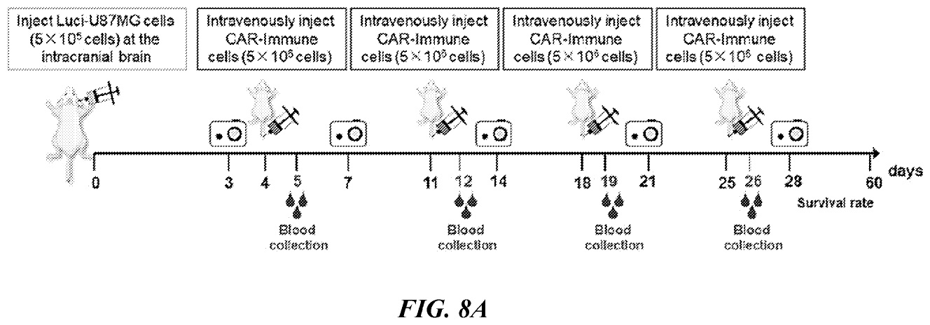

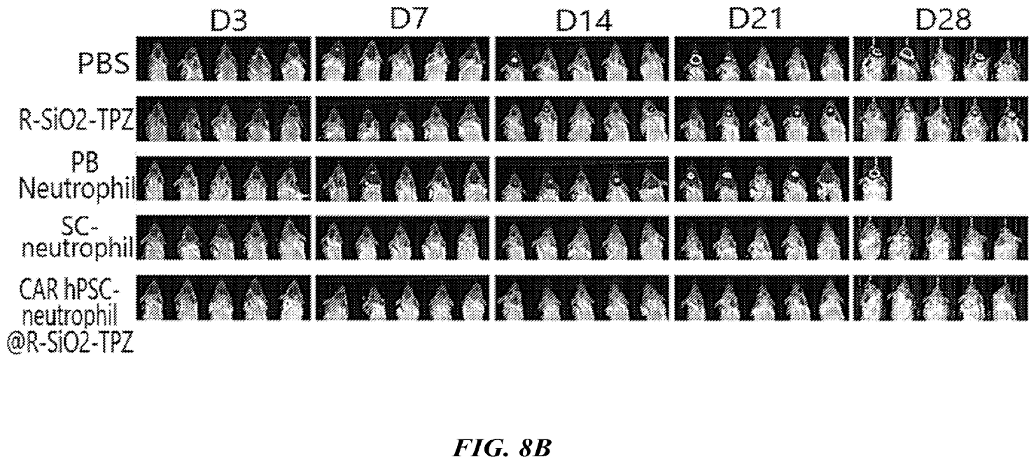

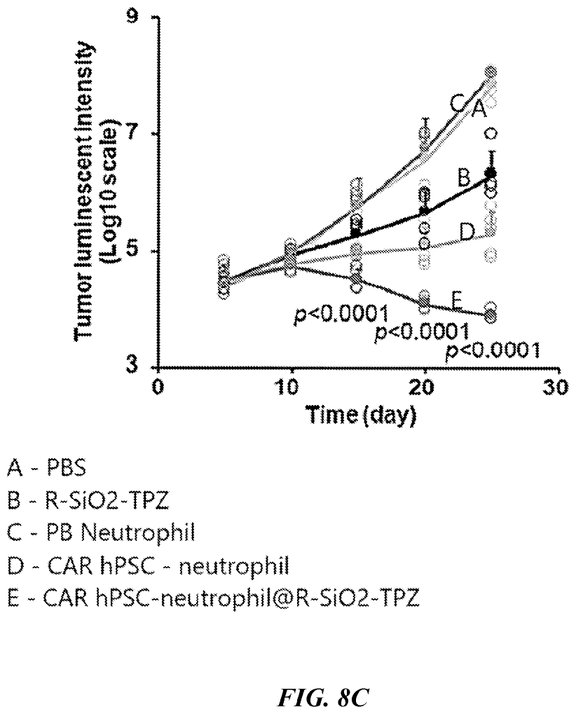

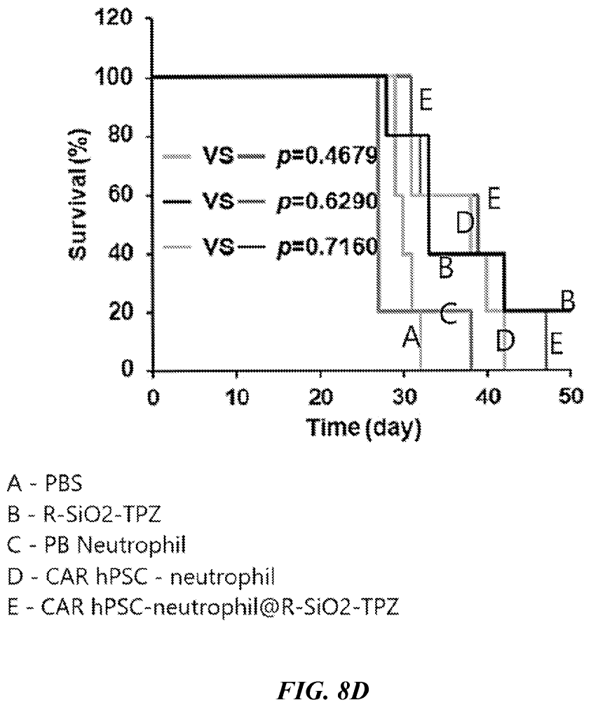

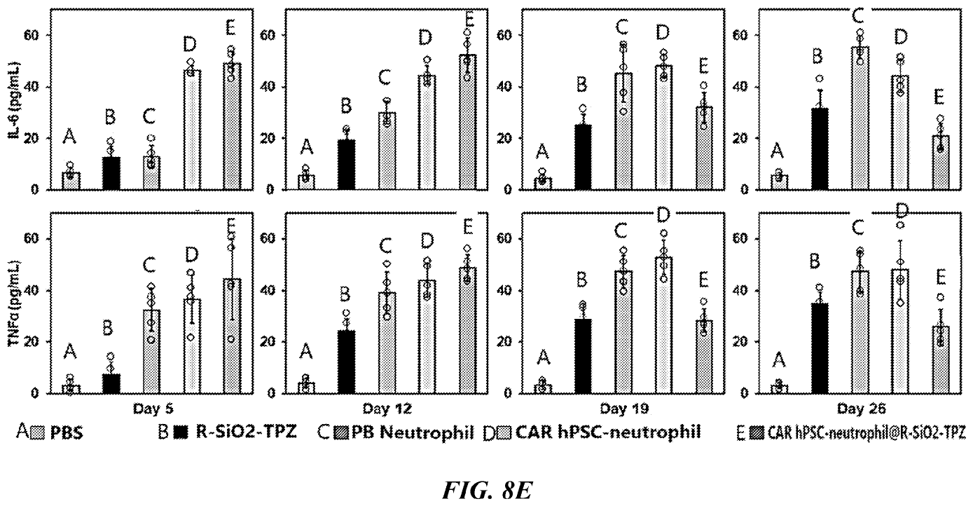

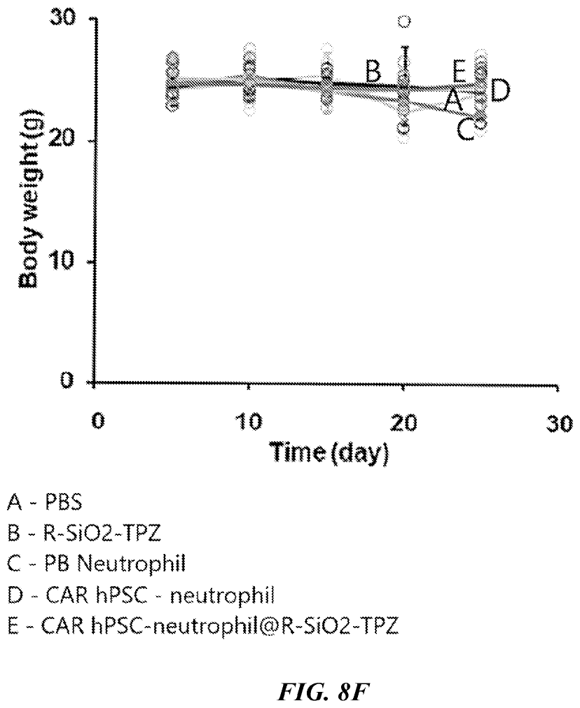

To determine the therapeutic efficacy of R—SiO₂-TPZ NP-loaded CAR-neutrophils, an in situ xenograft model of GBM was established in immunodeficient NOD.Cg-RAG^{tm1Mom}IL2rg^{tm1Wjt}/SzJ (NRG) mice using luciferase-expressing U87MG cells. FIG. 8A outlines the protocol of the study.

Briefly, in situ xenograft murine models were constructed via intracranial injection of 5×10⁵ luciferase-expressing GBM cells into the brain of immunodeficient mice. Namely, 5×10⁵ luciferase (Luci)-expressing U87MG cells were stereotactically implanted into the right forebrain of NRG mice. All mouse experiments were approved by the Purdue Institutional Animal Care and Use Committee (PACUC). The immunodeficient NOD.Cg-RAG1tm1MomIL2rgtm1Wjl/SzJ (NRG) mice were bred and maintained by the Biological Evaluation Core at Purdue University Institute for Cancer Research. All the female mice used in this study were 6- to 10-week-old. Mice were housed in pathogen free and ventilated cages, and allowed free access to autoclaved food and water, in a 12-hour light/dark cycle, with room temperature at 21±2 degree and humidity between 45 and 65%.

The tumor-bearing mice were intravenously administrated 5×10⁶ neutrophils at day 4, day 11, day 18, and day 25, and blood samples were collected from these mice at day 5, day 12, day 19, and day 26. Tumor burden was monitored by bioluminescence imaging (BLI) system (Spectral Ami Optical Imaging System; Spectral Instruments Imaging, Tucson, AZ) (FIGS. 8B and 8C), and body weights of experimental mice were measured once per week (FIG. 8F). Collected blood cells were stained with CD45 and analyzed in an Accuri C6 plus flow cytometer (Beckton Dickinson & Company, Franklin Lakes, NJ). Blood samples were also subjected for enzyme-linked immunosorbent assay (ELISA) to measure human TNFα and IL-6 cytokine release (Invitrogen, Waltham, MA). At the end of treatment, tumors were collected for H&E staining. For in vivo biodistribution analysis, fluorescence images were captured by the Spectral Ami Optical Imaging System 3 and 24 hours after intravenous injection of Cy5 (Lumiprobe)-labeled neutrophils.

The experimental endpoint was defined as death, luciferase signal intensity in bioluminescence imaging higher than 10⁹ a.u., or experiencing neurological symptoms (i.e., inactivity, seizure, ataxia, and/or hydrocephalus). The mice bearing a tumor over 10⁹ a.u. or experiencing neurological symptoms were euthanized.

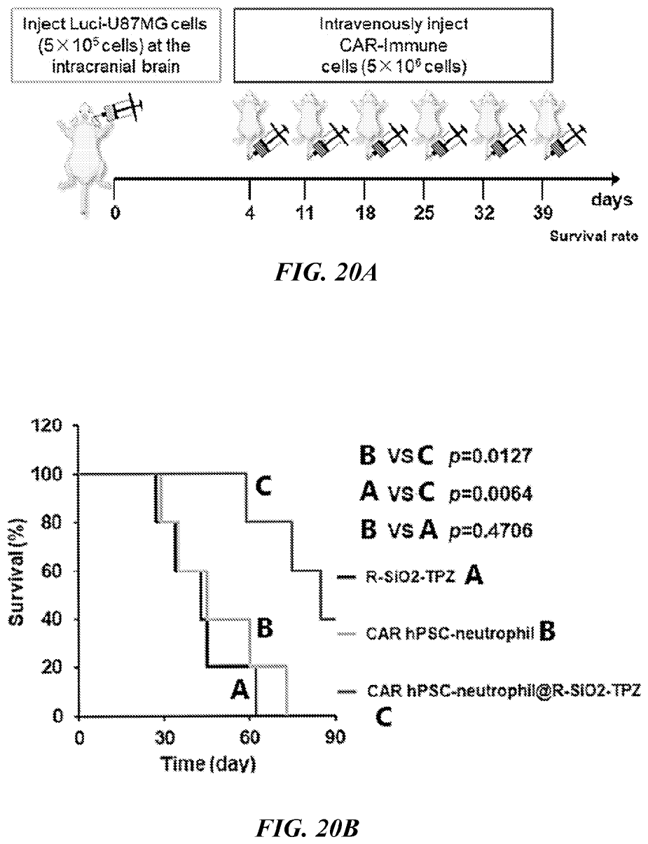

Tumor burden is quantified in FIGS. 20B and 20C. Compared to PBS or PB-neutrophil-treated mice, treatment with CAR-neutrophils and CAR-neutrophil-R—SiO₂-TPZ NPs displayed much higher anti-tumor cytotoxicity than any other experimental groups. On the contrary, PB-neutrophils significantly promoted tumor growth in the brain, resulting in the death of tumor-bearing mice as early as day 23 (FIG. 7D), suggesting that engineered neutrophils may pose additional risks.

Compared to PBS or PB-neutrophil treated mice, treatment with R—SiO₂-TPZ NPs, PB neutrophil@R—SiO₂-TPZ NPs, CAR-neutrophils, and CAR-neutrophil@R—SiO₂-TPZ NPs effectively slowed down tumor growth in host mice. As expected, CAR-neutrophil@R—SiO₂-TPZ NPs displayed much higher anti-tumor cytotoxicity than any other experimental groups. On the contrary, PB-neutrophils significantly promoted tumor growth in the host brains, resulting in the death of tumor-bearing mice as early as day 23 (FIG. 8D), suggesting that un-engineered neutrophils may pose additional risks.

Human cytokine release in the plasma of different experimental mouse groups was also measured (FIG. 8E). All non-PBS experimental groups produced detectable TNFα and IL-6 in the plasma from day 5 to day 26, suggesting the activation of human neutrophils upon tumor stimulation. Consistent with observed higher tumor growth rate, unmodified neutrophils gradually released more IL-6 and TNFα, which may lead to cytokine release syndrome in patients and require more in-depth safety studies with IL-6 blockers. Morris et al., Cytokine release syndrome and associated neurotoxicity in cancer immunotherapy, Nature Reviews Immunology 22: 85-96 (2022); Liang et al., Neutrophils promote the malignant glioma phenotype through S100A4, Clinical Cancer Research 20: 187-198 (2014). Notably, CAR-neutrophils@a R—SiO₂-TPZ NPs displayed decreased cytokine production ability at later time points (day 19 and day 26), suggesting a potentially low risk of cytokine release syndrome in patients treated with CAR-neutrophil based chemoimmunotherapy.

The biocompatibility of combinatory CAR-neutrophils and R—SiO₂-TPZ NPs was evaluated through weekly measurement of body weights and monitoring of pathological changes in major organs of mice. No detectable difference was observed in body weights between CAR-neutrophils@R-SiO2-TPZNP-treated mice and any other experimental groups, indicating minimal systemic toxicity and excellent biocompatibility of CAR neutrophil@R—SiO₂-TPZ NPs within 28 days of treatment (FIG. 8F).

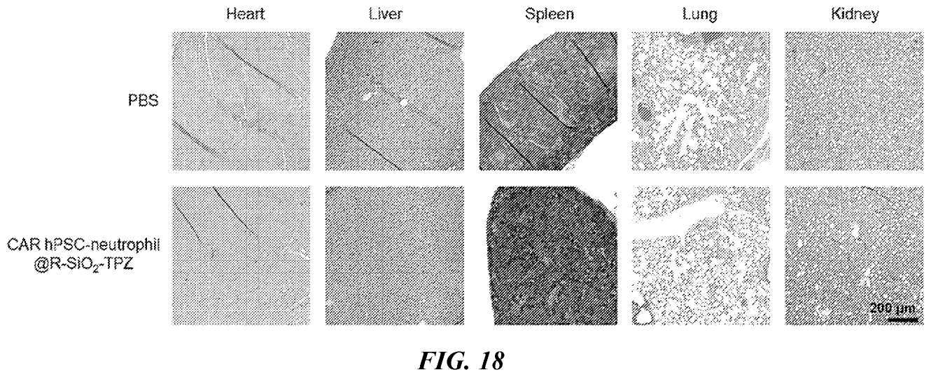

Histological analysis on major organs sliced from mice at day 30 showed that CAR neutrophil@R—SiO₂-TPZ NPs-treated mice did not cause appreciable abnormality or noticeable organ damage in heart, liver, spleen, lung, and kidney (FIG. 18), further confirming the safety of combinatory CAR-neutrophils and R—SiO₂-TPZ NPs.

While CAR-neutrophil@R—SiO₂-TPZ NPs significantly slowed down tumor growth in xenograft mice, the difference of animal survival in experimental groups of CAR-neutrophils, SiO₂-TPZ NPs and CAR-neutrophil@R—SiO₂-TPZ NPs is insignificant (p>0.05), which is possibly due to the death of short-lived neutrophils during cell preparation and injection.

Next, it was assessed if reduced cell preparation time and increased dosages of CAR-neutrophils and nanodrugs would make any difference in animal (with a focus on these three groups) (FIG. 20G). When systemically administered 6 times, CAR-neutrophil@R—SiO₂-TPZ NPs outperformed the other two groups in extending lifespan of tumor-bearing mice (FIG. 20H), whereas the difference of animal survival in groups of CAR-neutrophils and SiO₂-TPZ NPs remained insignificant. While a similar survival curve of the R—SiO₂-TPZ group was observed between these two independent animal studies, reduced time in cell isolation and preparation for injection from a total of ˜4 hours to 1 hour during the first 4 neutrophil doses led to improved animal survival in CAR-neutrophil groups before day 32.

Collectively, the data supports the importance of neutrophil preparation and dosage optimization in the clinical application of neutrophil therapeutics. See Chang et al., CAR-neutrophil mediated delivery of tumor-microenvironment responsive nanodrugs for glioblastoma chemoimmunotherapy, Nature Communications 14: 2266 (2023).

### Example 8

Anti-PSMA CAR-Neutrophils Derived from hPSCs Specifically Recognized and Killed Prostate Cancer Cells

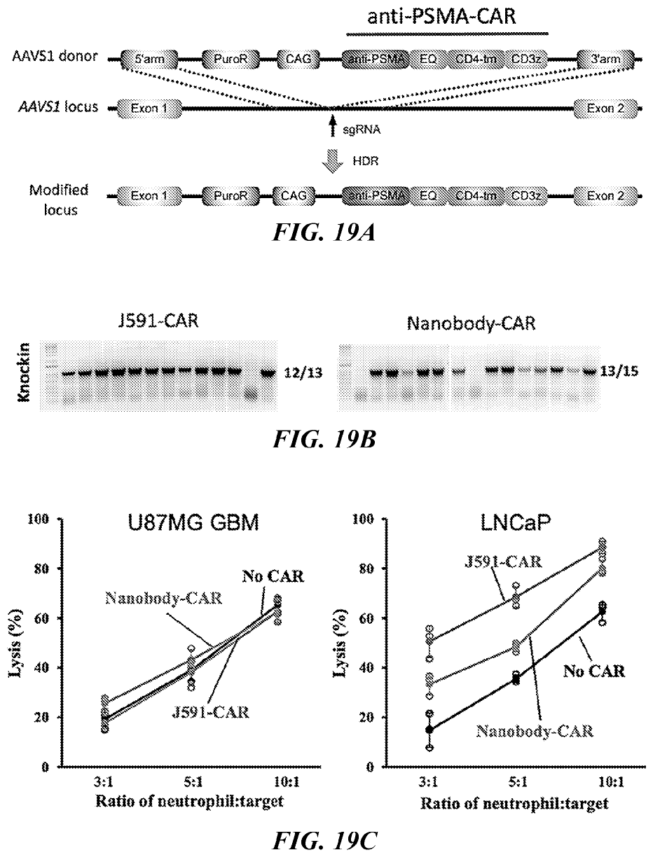

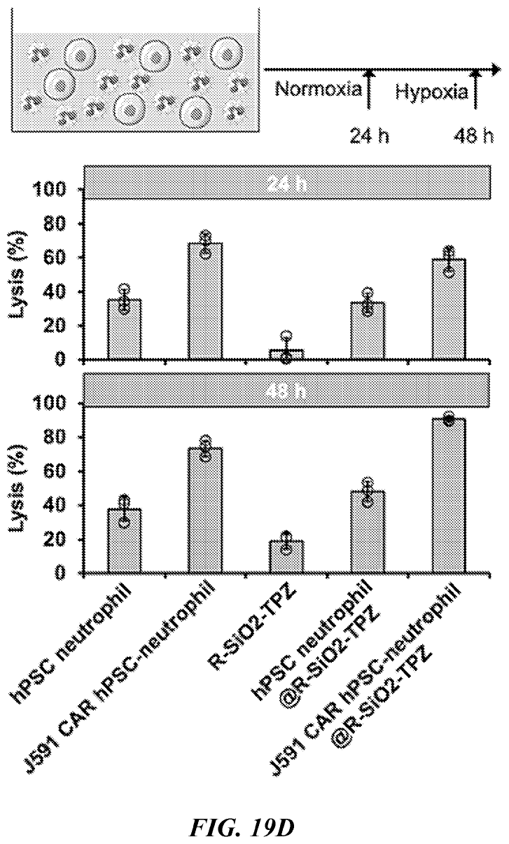

A PSMA-CAR was designed as shown in FIG. 19A and knocked in at the endogenous AAVS1 safe harbor locus via Cas9-mediated homology-directed repair (HDR). PSMA-CAR is composed of signal peptide, anti-PSMA J591 scFV or nanobody, IgG4-Fc (EQ), CD4 transmembrane (tm) and CD3ζ (CD3z). Genotyping of CAR knockin in hPSCs showed a target efficiency of 12 clones from a total of 13 and 13 clones from a total of 15, respectively (FIG. 19B). CAR-neutrophils were co-cultured with U87MG GBM and LNCaP prostate cancer cells at indicated cell ratios for 16 hours, and the cytotoxicity of neutrophils was calculated (FIG. 19C). Anti-PSMA J591 CAR-neutrophils were loaded with SiO₂-TPZ nanodrugs for enhanced antitumor cytotoxicity and tested in a hypoxia tumor model in vitro (FIG. 19D).

1. Chimeric antigen receptor (CAR)-expressing neutrophils loaded with nanoparticles comprising a drug, wherein the CAR-expressing neutrophils are differentiated from pluripotent stem cells (PSCs) engineered to express the CAR.

2. (canceled)

3. The CAR-expressing neutrophils of claim 1, wherein the PSCs are human PSCs (hPSCs).

4. (canceled)

5. The CAR-expressing neutrophils of claim 1, wherein the nanoparticles comprise one or more of rough silica nanoparticles, cytosine arabinoside-based liposomes, polyamidoamine dendrimer-albumin nanoparticles, and/or fullerene.

6. The CAR-expressing neutrophils of claim 5, wherein the rough silica nanoparticles are biodegradable mesoporous organic silica.

7. The CAR-expressing neutrophils of claim 1, wherein the drug is a prodrug, a chemotherapeutic drug, or a radiosensitizer.

8. The CAR-expressing neutrophils of claim 1, wherein the drug is:
a prodrug activated by hypoxic conditions, acidic pH, an enzyme, or irradiation;
tirapazamine, temozolomide, climacostol, or indole-3-acetic acid; or
selected from the group consisting of everolimus, bevacizumab, belzutifan, carmustine, naxitamab-gqgk, and lomustine.

9.-10. (canceled)

11. The CAR-expressing neutrophils of claim 1, wherein the CAR comprises;
a 36-amino acid glioblastoma (GBM)-targeting chlorotoxin peptide;
a CD32a transmembrane domain or a CD4 transmembrane domain; and
a CD3ζ intracellular domain and/or a CD32aγ intracellular signaling domain or a CD16 intracellular signaling domain.

12.-16. (canceled)

17. The CAR-expressing neutrophils of claim 1, wherein the CAR comprises:
a 36-amino acid GBM-targeting chlorotoxin peptide,
a natural killer group 2D (NKG2D) transmembrane domain,
a 2B4 co-stimulatory domain, and
a CD3ζ intracellular signaling domain.

18. The CAR-expressing neutrophils of claim 1, wherein the CAR comprises:
an IL-13 receptor α 2 (IL-13Rα2)-targeted quadruple mutant IL-13 (TQM13) T-CAR, a GD2-targeting single chain variable fragment (scFV), a human epidermal growth factor receptor 2 (HER2)-targeting scFV, a vIII mutant epidermal growth factor receptor (EGFRvIII)-targeting scFV, or other glioma-targeting scFV,
a CD4 transmembrane domain, and
a CD3ζ intracellular signaling domain.

19. The CAR-expressing neutrophils of claim 1, wherein the neutrophils have an anti-tumor N1 phenotype.

20. The CAR-expressing neutrophils of claim 18, wherein the neutrophils exhibit anti-glioblastoma activity in a hypoxic tumor microenvironment.

21. The CAR-expressing neutrophils of claim 1, wherein the CAR is encoded by SEQ ID NO: 2, SEQ ID NO: 3, SEQ ID NO: 4, or a functional variant of SEQ ID NO: 2, 3 or 4.

22.-44. (canceled)

45. A pharmaceutical composition comprising:
chimeric antigen receptor (CAR)-expressing neutrophils loaded with nanoparticles comprising a drug, wherein the CAR-expressing neutrophils are differentiated from pluripotent stem cells (PSCs) engineered to express the CAR; and
a pharmaceutically acceptable carrier and/or diluent.

46. The pharmaceutical composition of claim 45, further comprising a pharmaceutically acceptable excipient.

47. (canceled)

50. A method of treating cancer in a subject comprising administering to the subject a first therapy comprising a therapeutically effective amount of:
a population of CAR-expressing neutrophils loaded with nanoparticles comprising a drug, wherein the CAR-expressing neutrophils are differentiated from pluripotent stem cells (PSCs) engineered to express the CAR; or
a pharmaceutical composition comprising CAR-expressing neutrophils loaded with nanoparticles comprising a drug, wherein the CAR-expressing neutrophils are differentiated from PSCs engineered to express the CAR;
whereupon the subject is treated for cancer.

51. (canceled)

52. The method of claim 50, wherein the cancer is glioblastoma or a prostate cancer.

53. (canceled)

54. The method of claim 50, wherein administering the first therapy comprises a delivery route selected from the group consisting of intravenous, intraperitoneal, intramuscular, intradermal, subcutaneous, intrathecal, intraosseous, and a combination of any of the foregoing.

55. The method of claim 50, further comprising administering a second therapy to the subject.

56. The method of claim 55, wherein the second therapy comprises surgical removal of cancerous cells from the subject, or a chemotherapy, radiotherapy or both.

57. (canceled)

58. The method of claim 50, further comprising imaging a cancer in the subject prior to or during administering the first and/or second therapies.

59. The method of claim 55, wherein the first and second therapies are administered sequentially and/or alternatively.

60.-73. (canceled)


SEQUENCE LISTING (8 sequences)

SEQ ID NO: 1 - DNA, 11501 bp, synthetic construct
  source 1..11501: mol_type = other DNA
ctagtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccrggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacgccaccatgcttctcctggtgacaagccttctgctctgtgagttaccacacccagcattcctcctgatcccaatgtgcatgccttgcttcaccaccgaccaccagatggccagaaagtgcgatgactgctgcggcggcaagggcagaggcaagtgctacggcccccagtgtctgtgcagagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatggccctgattgtgctggggggcgtcgccggcctcctgcttttcattgggctaggcatcttcttcagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacgcgtgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggcgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgaatactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaaggg

SEQ ID NO: 2 - DNA, 11509 bp, synthetic construct
  source 1..11509: mol_type = other DNA
ctagctagtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggggggggggggggggggggggggsggggsggggsgrggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacgccaccatgcttctcctggtgacaagccttctgctctgtgagttaccacacccagcattcctcctgatcccaatgtgcatgccttgcttcaccaccgaccaccagatggccagaaagtgcgatgactgctgcggcggcaagggcagaggcaagtgctacggcccccagtgtctgtgcagagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatcattgtggctgtggtcattgcgactgctgtagcagccattgttgctgctgtagtggccttgatctacagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacgcgtgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggcgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgaatactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaaggga

SEQ ID NO: 3 - DNA, 11401 bp, synthetic construct
  source 1..11401: mol_type = other DNA
ctagctagtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccggggggggggggggggggsggggggsggggsggggsgrggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacgccaccatgcttctcctggtgacaagccttctgctctgtgagttaccacacccagcattcctcctgatcccaatgtgcatgccttgcttcaccaccgaccaccagatggccagaaagtgcgatgactgctgcggcggcaagggcagaggcaagtgctacggcccccagtgtctgtgcagagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaaacgcgtgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggcgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgaatactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaaggga

SEQ ID NO: 4 - DNA, 11779 bp, synthetic construct
  source 1..11779: mol_type = other DNA
ctagtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggggcsccrgggggggggggggggggggggggggggggggggggggggggggggggggggggggggcgrggggsggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacgccaccatgcttctcctggtgacaagccttctgctctgtgagttaccacacccagcattcctcctgatcccaatgtgcatgccttgcttcaccaccgaccaccagatggccagaaagtgcgatgactgctgcggcggcaagggcagaggcaagtgctacggcccccagtgtctgtgcagagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataacagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacgcgtgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggcgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgaatactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactag

SEQ ID NO: 5 - DNA, 25 bp, synthetic construct
  source 1..25: mol_type = other DNA
ctgtttccccttcccaggcaggtcc

SEQ ID NO: 6 - DNA, 25 bp, synthetic construct
  source 1..25: mol_type = other DNA
tcgtcgcgggtggcgaggcgcaccg

SEQ ID NO: 7 - DNA, 20 bp, synthetic construct
  source 1..20: mol_type = other DNA
cggttaatgtggctctggtt

SEQ ID NO: 8 - DNA, 20 bp, synthetic construct
  source 1..20: mol_type = other DNA
gagagagatggctccaggaa